SEQUENCE LISTING (132 sequences)

SEQ ID NO: 1 - RNA, 111 nt, Arabidopsis thaliana
  stem_loop (1)..(111)
gcaccaggggugcuugaaccaggauagccugcgaaaaggcgggcuauccgggaccaggcugagaaagucccuuugaaccugaacaggguaaugccugcgcagggagugugc

SEQ ID NO: 2 - RNA, 112 nt, Brassica sativa
  stem_loop (1)..(112)
  stem_loop (1)..(112)
gcaccaggggugcuugaaccaggcuagccugcuaaagagcgggcuauccugggaacaggcugagaaagucccuuugaaccugaacaggguaaugccugcgcagggagugugc

SEQ ID NO: 3 - RNA, 112 nt, Brassica oleracea
  stem_loop (1)..(112)
gcaccaggggugcuugaaccaggcuagccugcuaaaaagcgggguagccugggaacaggcugagaaagucccuuugaaccugaacaggguaaugccugcgcagggagugugc

SEQ ID NO: 4 - RNA, 111 nt, Boechera stricta
  stem_loop (1)..(111)
gcaccaggggugcuugaaccaggcuagcccgugaaagagcgggcuaucugggaccaggcugagaaagucccuuugaaccugaacaggguaaugccugcgcagggagugugc

SEQ ID NO: 5 - RNA, 121 nt, Carica papaya
  stem_loop (1)..(121)
gcaccaggggugccuguaucugccaagcaauagcuuuuaauuaauggcuacggugaaagcagaucaggcugagaaagucccuuugaaccugaacaggauaaugccugcguagggagugugc

SEQ ID NO: 6 - RNA, 116 nt, Citrus sinensis
  stem_loop (1)..(116)
gcaccaggggugccuguaucugcuuuaacacuagccaauuggccaggauacgggcagagcaggcugagaaagucccuuugaaccugaacagguuaaugccugcguagggagugugc

SEQ ID NO: 7 - RNA, 123 nt, Nicotiana tabacum
  stem_loop (1)..(123)
gcaccaggggugccugugucagcuucaaaaaccuggccuuauuagccagguuauacgcugacugaacaggcugagaaagucccuuugaaccugaacaggauaauuccugcguagggagugugc

SEQ ID NO: 8 - RNA, 123 nt, Nicotiana benthamiana
  stem_loop (1)..(123)
gcaccaggggugccugugucagcuucaaaaaccuggccuuauuagccagguuauaugcugacugaacaggcugagaaagucccuuugaaccugaacaggauaauuccugcguagggagugugc

SEQ ID NO: 9 - RNA, 118 nt, Populus trichocarpa
  stem_loop (1)..(118)
gcaccaggggugccugcguccugcuucaaaacuggccaucuggcuagaggaggcagcuggccaggcugagaaagucccuuugaaccugaacaggauaaugccugcguagggagugugc

SEQ ID NO: 10 - RNA, 116 nt, Lotus japonicus
  stem_loop (1)..(116)
gcaccaggggugccugaaucugcuugagcuuuggcuaauaagcuggaaagcuagcagaucaggcugagacagucccuuugaaccugaucagggugaugccugcguagggagagugc

SEQ ID NO: 11 - RNA, 97 nt, Lycopersicon esculentum
  stem_loop (1)..(97)
gcaccgggggugucugugucagcuucaaaugcugacugaucaggcugagaaagucccuuugaaccugaacgggauaauuccugcguagggagcgugc

SEQ ID NO: 12 - RNA, 97 nt, Solanum tuberosum
  stem_loop (1)..(97)
gcaccgggggugucugugucagcuucaaaugcugacugaucaggcugagaaagucccuuugaaccugaacgggauaauuccugcguagggagugugc

SEQ ID NO: 13 - RNA, 95 nt, Ocimum basilicum
  stem_loop (1)..(95)
gcaccgggggugucuguauccgcuccgucggggcggugcaggcugagaaagucccuuugaaccugaacaggauaaugccugcguagggagugugc

SEQ ID NO: 14 - RNA, 110 nt, Ipomoea nil
  stem_loop (1)..(110)
gggugucuguaucugcuccagcccuggcucccucggccaggaugcuggcugaaacaggcugagaaagucccuucgaaccugaacaggauaaugccugcguaggagcgugc

SEQ ID NO: 15 - RNA, 117 nt, Vitis vinifera
  stem_loop (1)..(117)
gcaccaggggugucugcaucugcuuuugcacuggcaauuuggccaaggaugcaagcagaucaggcugagaaagucccuucgaaccugaacaggauaaugccugcguagggagugugc

SEQ ID NO: 16 - RNA, 118 nt, Oryza sativa
  stem_loop (1)..(118)
gcaccaggggugccuguauucucaacgaucugaaggccucuuggccuggauuguugugaauugggcugagaaagucccuuugaaccugaacaggauaaugccugcgaagggagugugc

SEQ ID NO: 17 - RNA, 118 nt, Poa secunda
  stem_loop (1)..(118)
gcaccaggggugccuguauucucaacaaucugaagggcccuuggccuggauuguugugauaugggcugagaaagucccuuugaaccugaacaggauaaugccugcguagggagugugc

SEQ ID NO: 18 - RNA, 118 nt, Triticum aestivum
  stem_loop (1)..(118)
gcaccaggggugcuuguauucccaacgaucuuaaggccccuuggccuggauuguugcgauaugggcugagaaagucccuuugaaccugaacaggauaaugccugcgaagggagugugc

SEQ ID NO: 19 - RNA, 118 nt, Hordeum vulgare
  stem_loop (1)..(118)
gcaccaggggugcuuguauucccaacgaucugaaggccccuuagccuggaucguugcgauaugggcugagaaagucccuuugaaccugaacaggauaaugccugcgaagggagugugc

SEQ ID NO: 20 - RNA, 118 nt, Sorghum bicolor
  stem_loop (1)..(118)
gcaccaggggugccuguguucucaacagucugagagccucuuagccuggacuguugugagaugggcugagaaagucccuuugaaccugaacaggauaaugccugcgaagggagugugc

SEQ ID NO: 21 - RNA, 115 nt, Pinus taeda
  stem_loop (1)..(115)
gcaccaggggugcuugcccuguuuacaccgcuagcgaagcugggguuguugacaggagcaggcugagaaagucccuuugaaccugagcaggauaaugccugcguagggagugugc

SEQ ID NO: 22 - RNA, 114 nt, Physcomitrella patens
  stem_loop (1)..(114)
gcaccaggggugcuugcaugaugaagcagcgcaaucuauuugcgucgauuaucaaugcaggcugagagagucccuucgcaccugaacagguuaauaccugcguagggagcgugc

SEQ ID NO: 23 - RNA, 80 nt, Physcomitrella patens
  stem_loop (1)..(80)
gcgccgggggugcuugcaucuagcaggcugagagagucccuuaaaaccugaucaggguaauaccugcgaagggagugugc

SEQ ID NO: 24 - RNA, 80 nt, Physcomitrella patens
  stem_loop (1)..(80)
gcaccgggggugcuugcuuugagcaggcugagagagucccuuaaaaccugaucaggauaauaccugcgaagggagugugc

SEQ ID NO: 25 - RNA, 17 nt, Artificial Sequence
  consensus sequence of TPP riboswitch aptamers
gcaccrggggugyyugy

SEQ ID NO: 26 - RNA, 58 nt, Artificial Sequence
  consensus sequence of TPP riboswitch aptamers
ncaggcugagaaagucccuuugaaccugaacaggruaaurccugcgyagggagugugc

SEQ ID NO: 27 - RNA, 17 nt, Artificial Sequence
  consensus sequence of TPP riboswitch aptamer
nnnnnnggggngcynnn

SEQ ID NO: 28 - RNA, 16 nt, Artificial Sequence
  consensus sequence of TPP riboswitch aptamers
nnnrgcugagannnnn

SEQ ID NO: 29 - RNA, 49 nt, Artificial Sequence
  consensus sequence of TPP riboswitch aptamers
nnnnnnacccnynraaccugaucyrgnuraurcyrgcgnaggrannnnn

SEQ ID NO: 30 - RNA, 17 nt, Arabidopsis thaliana
  stem_loop (1)..(17)
gcaccaggggugcuuga

SEQ ID NO: 31 - RNA, 58 nt, Arabidopsis thaliana
  stem_loop (1)..(58)
ccaggcugagaaagucccuuugaaccugaacaggguaaugccugcgcagggagugugc

SEQ ID NO: 32 - RNA, 17 nt, Arabidopsis thaliana
  stem_loop (1)..(17)
gcaccaggggugcuuga

SEQ ID NO: 33 - RNA, 58 nt, Arabidopsis thaliana; identical to SEQ ID NO: 31
  stem_loop (1)..(58)

SEQ ID NO: 34 - RNA, 8 nt, Arabidopsis thaliana
  misc_signal (1)..(8): splice site
cuguuggu

SEQ ID NO: 35 - RNA, 7 nt, Brassica sativa
  misc_signal (1)..(7): splice site
uguuggu

SEQ ID NO: 36 - RNA, 8 nt, Nicotiana tabacum
  misc_signal (1)..(8): splice site
uguuaggu

SEQ ID NO: 37 - RNA, 8 nt, Nicotiana benthamiana
  misc_signal (1)..(8): splice site
uguuaggu

SEQ ID NO: 38 - RNA, 7 nt, Lycopersicon esculentum
  misc_signal (1)..(7): splice site
guuaggu

SEQ ID NO: 39 - RNA, 18 nt, Oryza sativa
  misc_signal (1)..(18): splice site
ccuauguuaggagguggu

SEQ ID NO: 40 - RNA, 9 nt, Ocimum basilicum
  misc_signal (1)..(9)
ugcauuggu

SEQ ID NO: 41 - RNA, 10 nt, Arabidopsis thaliana
  stem_loop (1)..(10)
gacaagucca

SEQ ID NO: 42 - RNA, 9 nt, Brassica sativa
  stem_loop (1)..(9)
acaagucca

SEQ ID NO: 43 - RNA, 9 nt, Nicotiana tabacum
  stem_loop (1)..(9)
acaagucca

SEQ ID NO: 44 - RNA, 9 nt, Nicotiana benthamiana
  stem_loop (1)..(9)
acaagucca

SEQ ID NO: 45 - RNA, 8 nt, Lycopersicon esculentum
  stem_loop (1)..(8)
caagucca

SEQ ID NO: 46 - RNA, 11 nt, Oryza sativa
  stem_loop (1)..(11)
ggacaagucca

SEQ ID NO: 47 - RNA, 9 nt, Ocimum basilicum
  stem_loop (1)..(9)
acaagucca

SEQ ID NO: 48 - DNA, 758 bp, Arabidopsis thaliana
  Unsure (1)..(758): genomic DNA
agctgctcagaaataaaaggtcagtatgtttagactgttagtcgttgctttctcaacaaacatgttagttactgcatgctagtataaaatcattcaggtttataatcttttcttaaatctgcaacatatggtcaactcttaaatgagtccttactgtgatctttgttttttatcgtgtttctttttcttctgctgcatcaggcaaatgttttaaacaagaccttgcttacccaagtcttggtgcctgttggactatacctggataaaggcacaaactgttggtaagcttagtagtctctatgtcatgttacttttagaactatctatgttgtctgttcatttgagtcagagtcagcaataaagacaatctaagttgatgtttcaatacttttttgtgtgatttggttggtgaattgacatgcaaaagcaccaggggtgcttgaaccaggatagcctgcgaaaaggcgggctatccgggaccaggctgagaaagtccctttgaacctgaacagggtaatgcctgcgcagggagtgtgcagttttttttttttcctgtagctttctaaaggagaagaagctactgttgccgctcgagtctcgttccacggttttcaacagttagtttcttatgagctaagagattcagcttaattggcttacagccataaaagaagtctttaactgatgcactaagtcactaacagtagggaataattcaatcaaaaaatcatccagattgataaaaatgcatttgcacc

SEQ ID NO: 49 - DNA, 555 bp, Brassica sativa
  Unsure (1)..(555): genomic DNA from Brassica sativa
gccagagagctatgtcaaggctgctaagaagtaaaaaggtcagtatccttagtggttattacttatacaaacatgttagttactcacatatggtcaaaatgtgtctttttctgtgagctctgcctgttgtcatctttcttttgcttatgcttccttagggaaagggtgggaacaagacctcgcttacacaagtcttggtgcctgttggactatacctgaataaggcacaaaatgttggtaagctttagtagtctctctgtctgttaaactttagaactatctatggtttatgtttcttctgtttgttcctttgagtctgagtcagtaataaaaaagacaaactgagttgatgttttaaatactttacatgtgatttggattgtgaattgacatagaaagcaccaggggtgcttgaaccaggctagcctgctaaagagcgggctatcctgggaacaggctgagaaagtccctttgaacctgaacagggtaatgcctgcgcagggagtgtgcagttttcttttttttcttttctataggagatgaagcttttctgct

SEQ ID NO: 50 - DNA, 1197 bp, Nicotiana tabacum
  Unsure (1)..(1197): genomic DNA
accggaaaattacatcaactctatgaaaagctaaaggtgagtgttacattggattttctcttgacattgttgtttttgcaagaaggggagccttggcgtaactggtaaagttgttgtcatgtgaccaggaggtcacgggttcgagccgcgcaaacaacctcttgtagaaatgcagggtaaggctgcgtacagtagacccttgtggtccggcccttccctagacaccgcacatagcgggaacttagtgcatcgggctgccctttattattgtttctgcaagttctttaaaggtagagatatgttatatccaaaaaatgattacctctgttaacctgcaacttaactcaacaaacactctattttcgactaactctatttaaatctaaaataagaaaatttctcatcacttagattatacaaataagtggtaggagatgaaagcatggttatctaatagagggcttcttttttgcaaacatgttcttttctcgacgtcgggttatccaataggagttgttgtagttttcgtagttgcaggaaccttttttttccctccgcaacgtgttaccttactgaatgcaattgcaggacttcatagattgaaaactggatcaagagctgtctgaagtgatcacgctccatcggccagtcaagaaaggagggtgtaggagcttcattatcaagtgttaggtaagtatctaggtttggtatgtttaataacttgaaaacaacagccttgttcagaaaagtactccttatcagtaataagaaatcacgtattttattgtattctgtctcttatatctttgacaatttattgacacgagaaagcaccaggggtgcctgtgtcagcttcaaaaacctggccttattagccaggttatacgctgactgaacaggctgagaaagtccctttgaacctgaacaggataattcctgcgtagggagtgtgcatttttttgttgcttgcacagggatggagattcttccatggttcaaattaagctgatcctgccctttgctaggaccagttatacattatattcaagatcaggggtgtattcagtagtagagttatgggttcatttgaagttttgaacttaacttttagcctgaatttatctgtgttaggcgcatggcctttcttcggattaggtccggggcacatttcgggcgtgtacaatttttgaccactgc

SEQ ID NO: 51 - DNA, 1175 bp, Nicotiana benthamiana
  unsure (1)..(1175): genomic DNA
gccaaagttttgttctatgaagataactgaagatataaggaagtatgctgaaactcacggttatggaagtgcagaggaagcaatcctccgcggcatggatgctatgagtgcagaatttcaagctgcaaagaaaaccattagcggggaacaacatggtgaggttggtggtgaaatctacttgccggaaaattacatcaactctttgaagagctaaaggtgagcgttcaatcggattttctcttgacattgttgtttctgcaagttctttaaaggtagagatatatccaagaaattattacctttgtgaacctgcaacttaactcaacaaactctctatttccgactacctatatttaaatctaaataagaaaatttcccatcatttagatagagtatacaataaagtggtcggagatgaaagcatgggttattcaatagagggcttcttttttgcaaacatgttcttttctcgacgtcaggttatccaataggagttgttgtacttttcatagttgcaagatttttcccccctccacaacgtcttaccttactgaatgcaattgcaggacttcatagatggaaaactggatcaagagctgtctgaagtgatcacgctccatcggccagtcaagaaaggagggtgtaggagcttcattatcaagtgttaggtaagtatctaggtttggtatgttaaataacttgaaaacaacagccttgttcaaagaaattactcttcttatcagtaataagaaatcgcgttttttattgtattctgtcttttatatcttcaacaatttgttgacacgagaaagcaccaggggtgcctgtgtcagcttcaaaaacctggccttattagccaggttatatgctgactgaacaggctgagaaagtccctttgaacctgaacaggataattcctgcgtagggagtgtgcatttttttttttgttgcttgcacagggatggagattcttccatgcttcaaattaagctgatcctgccctttgccaggaccagtaatacattatattcaagatcaggggtgtattcagtagtagagttatgggttcatttgaagttttgaactcataacttttagcctgaatttatttgttaggcgcatggcctttcttcggattaggtccggggactgtgtgtccggggcacatttcgggc

SEQ ID NO: 52 - DNA, 922 bp, Lycopersicon esculentum
  Unsure (1)..(922): genomic DNA
gccagagaattacatcaactctttgaagagccaaaggtgagtattacattctgcaagtcctttgaagacactcgtataatgcagagtctttgtgtattccgattaaagctcctttcacttaccttaaagaatacaatttcaggaatgtgtaaagatcgaaaactggctcacagatttctgaagtgatcacaactccatcggccagtcaaggaggcggtagtaggagcctcattatcaagtgttaggtaagtatctagtttggtactgttaaataaactgagacaacagccttgttcaatgaaactctcctttgttagtaatgagaaaaacgcgtattttcttgtgttcagtcttttatatcttttaagttttattgacacgagaaagcaccgggggtgtctgtgtcagcttcaaatgctgactgatcaggctgagaaagtccctttgaacctgaacgggataattcctgcgtagggagcgtgcaatttcttttttcttgcttgcacagggatggcgattctccaaaatggttgaaaattcaaaattaatctgatcctgctctttgccaggaccagtttattcactgtctattcaagacaccatcttgtattgtttgtttttttggtgtcctgtaccgcattgaggcctgactaaatacagattcccgcactctgtacataacagagctcgaattcgacacctttgattggtagtaaaggggtatttatcactccatcacgacccttgttggtaaggcatattcgtgtatgcctctgagacgcgattggttacttacaattgaccaattttctcatggttagtctagctttggttggactattgaaacattcagtaatatgaatgactcaattgcatatataccttcatattttgaaatcaatattacgaatttaagatctgg

SEQ ID NO: 53 - DNA, 1224 bp, Oryza sativa
  unsure (1)..(1224): genomic DNA
ctatacagctcgcaaataaggttggtctttttcttatcatgtatagttcaccttgtgggagatgtttggactcttttgttctgaatatgtagggtttttaactcagtatgaagcaattagtcatctggagtaaaatggtctagtgccgtatttcaatcagttttacataaataagccattaatgtgagcttaccgtcttgaaacagcttttcagatgccttctctagcacttttgttgacccagtaacttcatacatgctactagttatatagcattagtgcttgttacaaatatttctgaattatcttgactaaataaatactactcgagtactatctaccattcagtaaacattctgctaatccttttactccctccatcccgaaaaaaatgaatctaggactggatgtgacacatttagtacaacgaatctggacagaggtatgtctagattcatagtactaggatgtgtcacaccagtactaggttggttttttatgggacggagggagtacatcagaaatttttcataaatctatgagaactgttgtaattttgactccacacactatcttcagttatatttcttaagggaaaattactctcacagcaatcctttgaaatgggtttgcatgtacatttgaaatttgacactaactgtaatagcactggatagctgctctgtttttatcaacttctacttgtaatttcagggaccttaataaacctttagtggcttggacttgtatgtgcattactgcaaaggcactaacttttatatcacaataacatgttctcgccttctcggaactcagaatacaatctccgtgcagatcgtcttggtgtccatctactccgggtggagcgtttatcagcagcactcctcaccatcctatgttaggaggtggtaagcagtataagtgccttgaataaagctggtgttgcttagctcaatttgttaattctgtcgtggcttgtaataaaattaaactctatatgtgatccttccgtgtcatattttctactctttgcatcactgtgggttgttagtaatgaaagttgcaccaggggtgcctgtattctcaacgatctgaaggcctcttggcctggattgttgtgaattgggctgagaaagtccctttgaacctgaacaggataatgcctgcgaagggagtgtgcatttctacttttatgtttccagggaactctcaacacaacccctt

SEQ ID NO: 54 - DNA, 1506 bp, Ocimum basilicum
  unsure (1)..(1506): genomic DNA
gcctgaggagtatgtgaaatctaaggcagtttgaaggtataacttgaaaacaatatatatttttcgctcgttcagtcaacttggaaatctatccaattgtttttcgggaaaacacggtaaattatcctattgtttcactaattaagggattttattccataatttgaaagttaaggtaaattatccttcatttacttctagtagggtatttttacccaaacggtagggtaaaataccctatcggaaacggtctggagtataatctacctaacttttaaattgcggtataaaatactttaactagtgaaataataagataatttaccataacttcaaattgtgggaaaatctcctaatttgtgaaacagtgggataattgacctaatttcccgtagtatttttatcaggcaaaaatatcccagactttcattctatcacggatttctatgccactatgctgacaatgttgagaacgtgtttattcagcgattaaatccacatggatgctgacgaggatgtacgacgtgtagttttttttattacacatgacacaaaacgtctttctgtattgtgtataataaaatgtaaaattacgtgtcgcacgtccccgtcagcatccacgtggatttaacggttgaataaacacattttgaacactattagcgtaaagtttgggatactttttcctaataaaaatactatgagatagatttccaagctgaccgaaagatatgtcatgttttcccttctttctattcgttcgatcttcaacttctaacgcttcgactgtttcgattctccagagtacgaggcggtcgatccagagaagaaataataaaagttgacattggctagagagctatgatgcatggttagccaaagttgaggattctgaggtttcagaaacctcttcgtgcattggtaagtaaagtagtaggttgaagtattaagaagtttgttgatcttgtttgcttaaatagacatgtagcaataaaagtgcattttctctggattctctcgatatcgcgcgatcttttatcgacttgttgattgatgcgaaaacgcaccgggggtgtctgtatccgctccgtcggggcggtgcaggctgagaaagtccctttgaacctgaacaggataatgcctgcgtagggagtgtgctatcttttctgttttcacagggttggaggtcgaattcaaatgcaccattgaagccatccgctccttcctcaacggaacaacatcgactataattcaatctcgtgtcgagtatactcgaaaattctatgatgccaatgtggatattgtccgccgtatgcacaaaagtggtgtggggttaataacttaatatgggcccactacttgtgcgtatgccgtgggcaatatccacactgacattatcggaatttttttatataaaaaacaaggctcttaaagatttagaagaaaaagttagaattttgcttattcttttaaatacttttttaatgaggtttcagtttcaaggac

SEQ ID NO: 55 - DNA, 56 bp, Artificial Sequence
  primer
gctgtcaacgatacgctacgtaacggcatgacagtgtttttttttttttttttttt

SEQ ID NO: 56 - DNA, 34 bp, Artificial Sequence
  primer
agctgtcgacaaggcaaatgttttaaacaagacc

SEQ ID NO: 57 - DNA, 34 bp, Artificial Sequence
  primer
agctgtcgacggtgcaaatgcatttttatcaatc

SEQ ID NO: 58 - DNA, 22 bp, Artificial Sequence
  primer
cagtcacaaagcctacgatcaa

SEQ ID NO: 59 - DNA, 20 bp, Artificial Sequence
  primer
cggtgaagtaggtggagaaa

SEQ ID NO: 60 - DNA, 19 bp, Artificial Sequence
  primer
cgggatcactctcggcatg

SEQ ID NO: 61 - DNA, 23 bp, Artificial Sequence
  primer
gcacayttytgctcnatgtgygg

SEQ ID NO: 62 - DNA, 21 bp, Artificial Sequence
  primer
ggttcaaagggactttctcag

SEQ ID NO: 63 - DNA, 21 bp, Artificial Sequence
  primer
ctgagaaagtccctttgaacc

SEQ ID NO: 64 - DNA, 21 bp, Artificial Sequence
  primer
accgaaattctgctccatgaa

SEQ ID NO: 65 - DNA, 20 bp, Artificial Sequence
  primer
agcagaaaagcttcatctcc

SEQ ID NO: 66 - DNA, 23 bp, Artificial Sequence
  primer
gccaaagttttgttctatgaaaa

SEQ ID NO: 67 - DNA, 22 bp, Artificial Sequence
  primer
gcagtggtcaaaaattgtacac

SEQ ID NO: 68 - DNA, 22 bp, Artificial Sequence
  primer
gccaaagttttgttctatgaag

SEQ ID NO: 69 - DNA, 22 bp, Artificial Sequence
  primer
gcagtggtcaaaaattgtacac

SEQ ID NO: 70 - DNA, 22 bp, Artificial Sequence
  primer
tcctaagttttgctccatgaaa

SEQ ID NO: 71 - DNA, 23 bp, Artificial Sequence
  primer
ccagatcttaaattcgtaatatt

SEQ ID NO: 72 - DNA, 18 bp, Artificial Sequence
  primer
ttggcggcgaagaagacg

SEQ ID NO: 73 - DNA, 24 bp, Artificial Sequence
  primer
aaatctttaagagccttgtttttt

SEQ ID NO: 74 - DNA, 22 bp, Artificial Sequence
  primer
atgtgcaggtgatgaatgaagg

SEQ ID NO: 75 - DNA, 23 bp, Artificial Sequence
  primer
gtagaatggtgcctcgttacacc

SEQ ID NO: 76 - DNA, 24 bp, Artificial Sequence
  primer
ctgctcagaaataaaaggcaaatg

SEQ ID NO: 77 - DNA, 27 bp, Artificial Sequence
  primer
ctactaagcttaccaacagtttgtgcc

SEQ ID NO: 78 - DNA, 19 bp, Artificial Sequence
  primer
gcacaaactgttggggtgc

SEQ ID NO: 79 - DNA, 24 bp, Artificial Sequence
  primer
cattaccctgttcaggttcaaagg

SEQ ID NO: 80 - DNA, 26 bp, Artificial Sequence
  primer
aatacttttttgtgtgatttggttgg

SEQ ID NO: 81 - DNA, 18 bp, Artificial Sequence
  primer
agcctggtcccggatagc

SEQ ID NO: 82 - DNA, 30 bp, Artificial Sequence
  primer
ggtaataactgcatctaaagacagagttcc

SEQ ID NO: 83 - DNA, 17 bp, Artificial Sequence
  primer
ccacaaccgcttggtcg

SEQ ID NO: 84 - DNA, 23 bp, Artificial Sequence
  primer
gtgtctaccgactttggtcaagc

SEQ ID NO: 85 - DNA, 20 bp, Artificial Sequence
  primer
accccattcgttgtcgtacc

SEQ ID NO: 86 - DNA, 17 bp, Artificial Sequence
  primer
ctgctgcccgacaacca

SEQ ID NO: 87 - DNA, 21 bp, Artificial Sequence
  primer
gaactccagcaggaccatgtg

SEQ ID NO: 88 - DNA, 19 bp, Artificial Sequence
  primer
agacccacaaggccctgaa

SEQ ID NO: 89 - DNA, 18 bp, Artificial Sequence
  primer
cagctgcacgggcttctt

SEQ ID NO: 90 - DNA, 17 bp, Artificial Sequence
  primer
caagcgtttgaccggga

SEQ ID NO: 91 - DNA, 31 bp, Artificial Sequence
  primer
atgcgtcgacttatttctgagcagctttgac

SEQ ID NO: 92 - DNA, 17 bp, Artificial Sequence
  primer
gggtgcttgaaccagga

SEQ ID NO: 93 - DNA, 34 bp, Artificial Sequence; identical to SEQ ID NO: 57
  primer

SEQ ID NO: 94 - DNA, 38 bp, Artificial Sequence
  primer
taatacgactcactataggcaaactgttggggtgcttg

SEQ ID NO: 95 - DNA, 18 bp, Artificial Sequence
  primer
cacactccctgcgcaggc

SEQ ID NO: 96 - DNA, 35 bp, Artificial Sequence
  primer
taatacgactcactataggcacaaactgttggtaa

SEQ ID NO: 97 - DNA, 17 bp, Artificial Sequence
  primer
aaactgcacactccctg

SEQ ID NO: 98 - DNA, 28 bp, Artificial Sequence
  primer
agctggatccgcattccggtactgttgg

SEQ ID NO: 99 - DNA, 44 bp, Artificial Sequence
  primer
agctgtcgacttatacggctattccgcccttcttggcctttatg

SEQ ID NO: 100 - DNA, 29 bp, Artificial Sequence
  primer
agctggatccatgacttcgaaagtttatg

SEQ ID NO: 101 - DNA, 31 bp, Artificial Sequence
  primer
agctgtcgacttattgttcatttttgagaac

SEQ ID NO: 102 - DNA, 31 bp, Artificial Sequence
  primer
agctggatccatggtgagcaagggcgaggag

SEQ ID NO: 103 - DNA, 33 bp, Artificial Sequence
  primer
agctgtcgacttacttgtacagctcgtccatgc

SEQ ID NO: 104 - DNA, 28 bp, Artificial Sequence
  primer
agctggatccatggcctcctccgagaac

SEQ ID NO: 105 - DNA, 28 bp, Artificial Sequence
  primer
agctgtcgacctacaggaacaggtggtg

SEQ ID NO: 106 - DNA, 34 bp, Artificial Sequence
  primer
agctgtcgacattgaaacatcaacttagattgtc

SEQ ID NO: 107 - DNA, 30 bp, Artificial Sequence
  primer
agctgtcgacaggacttcatagatggaaaa

SEQ ID NO: 108 - DNA, 32 bp, Artificial Sequence
  primer
agctgtcgactaaaaaacgcgatttcttatta

SEQ ID NO: 109 - DNA, 27 bp, Artificial Sequence
  primer
agctgtcgacgcccgaaatgtgccccg

SEQ ID NO: 110 - DNA, 32 bp, Artificial Sequence
  primer
tccgggaccaggctgtcaaagtccctttgaac

SEQ ID NO: 111 - DNA, 32 bp, Artificial Sequence
  primer
gttcaaagggactttgacagcctggtcccgga

SEQ ID NO: 112 - DNA, 31 bp, Artificial Sequence
  primer
cctttgaacctgaactcggtaatgcctgcgc

SEQ ID NO: 113 - DNA, 31 bp, Artificial Sequence
  primer
gcgcaggcattaccgagttcaggttcaaagg

SEQ ID NO: 114 - DNA, 35 bp, Artificial Sequence
  primer
agctgtcgacaaggtcagtatgtttagactgttag

SEQ ID NO: 115 - DNA, 34 bp, Artificial Sequence
  primer
agctgtcgacctctccacctaaactcagattttg

SEQ ID NO: 116 - DNA, 38 bp, Artificial Sequence
  primer
agctgtcgacaccggtgagctcactagtaagcttagct

SEQ ID NO: 117 - DNA, 38 bp, Artificial Sequence
  primer
agctaagcttactagtgagctcaccggtgtcgacagct

SEQ ID NO: 118 - DNA, 32 bp, Artificial Sequence
  primer
tccgggaccaggctctctaagtccctttgaac

SEQ ID NO: 119 - DNA, 32 bp, Artificial Sequence
  primer
gttcaaagggacttagagagcctggtcccgga

SEQ ID NO: 120 - DNA, 19 bp, Artificial Sequence
  primer
gcaccagccgtgcttgaac

SEQ ID NO: 121 - DNA, 19 bp, Artificial Sequence
  primer
gttcaagcacggctggtgc

SEQ ID NO: 122 - DNA, 22 bp, Artificial Sequence
  primer
cacccttctccttctagtgaat

SEQ ID NO: 123 - DNA, 18 bp, Artificial Sequence
  primer
agctggagacaaacgaaa

SEQ ID NO: 124 - DNA, 21 bp, Artificial Sequence
  primer
atgtgcaggtgatgaatgaag

SEQ ID NO: 125 - DNA, 21 bp, Artificial Sequence
  primer
caaaggaccaagggtgtagaa

SEQ ID NO: 126 - DNA, 17 bp, Artificial Sequence
  primer
tggagtggtgtaacgag

SEQ ID NO: 127 - DNA, 18 bp, Artificial Sequence
  primer
gcgtcaatgtaatgttct

SEQ ID NO: 128 - DNA, 19 bp, Artificial Sequence
  primer
tctctgccgtttccaaatc

SEQ ID NO: 129 - DNA, 17 bp, Artificial Sequence
  primer
gatgtgctgtgcctgaa

SEQ ID NO: 130 - DNA, 19 bp, Artificial Sequence
  primer
gagcccaagtttttgaaga

SEQ ID NO: 131 - DNA, 19 bp, Artificial Sequence
  primer
ctaacagcgaaacgtccca

SEQ ID NO: 132 - DNA, 16 bp, Artificial Sequence
  primer
ccccaaccaagcccat

(19) United States
(12) Patent Application Publication
(10) Pub. No.: US 2010/0221821 A1
(43) Pub. Date: Sep. 2, 2010

(54) METHODS AND COMPOSITIONS RELATED TO RIBOSWITCHES THAT CONTROL ALTERNATIVE SPLICING AND RNA PROCESSING

(75) Inventors: Ronald R. Breaker, Guilford, CT (US); Andreas Wachter, Mannheim (DE)

Correspondence Address: PATENT CORRESPONDENCE;ARNALL GOLDEN GREGORY LLP, 171 17TH STREET NW, SUITE 2100, ATLANTA, GA, 30363, US

(73) Assignee: YALE UNIVERSITY, NEW HAVEN, CT (US)

(21) Appl. No.: 12/602,390

(22) PCT Filed: May 29, 2008
(86) PCT No.: PCT/US08/65112

§ 371 Date: May 14, 2010

Related U.S. Application Data
(60) Provisional application No. 60/932,164, filed on May 29, 2007.

(51) Int. Cl. C12N 15/74

(52) U.S. Cl. 435/320.1

(57) ABSTRACT

Disclosed are methods and compositions related to riboswitches that control alternative splicing.

### CROSS-REFERENCE TO RELATED APPLICATIONS

This application claims benefit of U.S. Provisional Application No. 60/932,164, filed May 29, 2007. U.S. Provisional Application No. 60/932,164, filed May 29, 2007, is hereby incorporated herein by reference in its entirety.

### STATEMENT REGARDING FEDERALLY SPONSORED RESEARCH

This invention was made with government support under Grant Nos. GM 068819, GM 07223 and DK 070270 awarded by the NIH and Grant No. MCB-0236210 awarded by the National Science Foundation. The government has certain rights in the invention.

### FIELD OF THE INVENTION

The disclosed invention is generally in the field of gene expression and specifically in the area of regulation of gene expression.

### BACKGROUND OF THE INVENTION

Precision genetic control is an essential feature of living systems, as cells must respond to a multitude of biochemical signals and environmental cues by varying genetic expression patterns. Most known mechanisms of genetic control involve the use of protein factors that sense chemical or physical stimuli and then modulate gene expression by selectively interacting with the relevant DNA or messenger RNA sequence. Proteins can adopt complex shapes and carry out a variety of functions that permit living systems to sense accurately their chemical and physical environments. Protein factors that respond to metabolites typically act by binding DNA to modulate transcription initiation (e.g. the lac repressor protein; Matthews, K. S., and Nichols, J. C., 1998, Prog. Nucleic Acids Res. Mol. Biol. 58, 127-164) or by binding RNA to control either transcription termination (e.g. the PyrR protein; Switzer, R. L., et al., 1999, Prog. Nucleic Acids Res. Mol. Biol. 62, 329-367) or translation (e.g. the TRAP protein; Babitzke, P., and Gollnick, P., 2001, J. Bacteriol. 183, 5795-5802). Protein factors respond to environmental stimuli by various mechanisms such as allosteric modulation or post-translational modification, and are adept at exploiting these mechanisms to serve as highly responsive genetic switches (e.g. see Ptashne, M., and Gann, A. (2002). Genes and Signals. Cold Spring Harbor Laboratory Press, Cold Spring Harbor, N.Y.).

In addition to the widespread participation of protein factors in genetic control, it is also known that RNA can take an active role in genetic regulation. Recent studies have begun to reveal the substantial role that small non-coding RNAs play in selectively targeting mRNAs for destruction, which results in down-regulation of gene expression (e.g. see Hannon, G. J. 2002, Nature 418, 244-251 and references therein). This process of RNA interference takes advantage of the ability of short RNAs to recognize the intended mRNA target selectively via Watson-Crick base complementation, after which the bound mRNAs are destroyed by the action of proteins. RNAs are ideal agents for molecular recognition in this system because it is far easier to generate new target-specific RNA factors through evolutionary processes than it would be to generate protein factors with novel but highly specific RNA binding sites.

Although proteins fulfill most requirements that biology has for enzyme, receptor and structural functions, RNA also can serve in these capacities. For example, RNA has sufficient structural plasticity to form numerous ribozyme domains (Cech & Golden, Building a catalytic active site using only RNA. In: The RNA World R. F. Gesteland, T. R. Cech, J. F. Atkins, eds., pp. 321-350 (1998); Breaker, In vitro selection of catalytic polynucleotides. Chem. Rev. 97, 371-390 (1997)) and receptor domains (Osborne & Ellington, Nucleic acid selection and the challenge of combinatorial chemistry. Chem. Rev. 97, 349-370 (1997); Hermann & Patel, Adaptive recognition by nucleic acid aptamers. Science 287, 820-825 (2000)) that exhibit considerable enzymatic power and precise molecular recognition. Furthermore, these activities can be combined to create allosteric ribozymes (Soukup & Breaker, Engineering precision RNA molecular switches. Proc. Natl. Acad. Sci. USA 96, 3584-3589 (1999); Seetharaman et al., Immobilized riboswitches for the analysis of complex chemical and biological mixtures. Nature Biotechnol. 19, 336-341 (2001)) that are selectively modulated by effector molecules.

Alternative splicing is a process which involves the selective use of splice sites on a mRNA precursor. Alternative splicing allows the production of many proteins from a single gene and therefore allows the generation of proteins with distinct functions. Alternative splicing events can occur through a variety of ways including exon skipping, the use of mutually exclusive exons and the differential selection of 5′ and/or 3′ splice sites. For many genes (e.g., homeogenes, oncogenes, neuropeptides, extracellular matrix proteins, muscle contractile proteins), alternative splicing is regulated in a developmental or tissue-specific fashion. Alternative splicing therefore plays a critical role in gene expression. Recent studies have revealed the importance of alternative splicing in the expression strategies of complex organisms.

Alternative splicing of mRNA precursors (pre-mRNAs) plays an important role in the regulation of mammalian gene expression. The regulation of alternative splicing occurs in cells of various lineages and is part of the expression program of a large number of genes. Recently, it has become clear that alternative splicing controls the production of proteins isoforms which, sometimes, have completely different functions. Oncogene and proto-oncogene protein isoforms with different and sometimes antagonistic properties on cell transformation are produced via alternative splicing. Examples of this kind are found in Makela, T. P. et al. 1992, Science 256:373; Yen, J. et al. 1991, Proc. Natl. Acad. Sci. U.S.A. 88:5077; Mumberg, D. et al. 1991, Genes Dev. 5:1212; Foulkes, N. S. and Sassone-Corsi, P. 1992, Cell 68:411. Also, alternative splicing is often used to control the production of proteins involved in programmed cell death such as Fas, Bcl-2, Bax, and Ced-4 (Jiang, Z. H. and Wu J. Y., 1999, Proc Soc Exp Biol Med 220: 64). Alternative splicing of a pre-mRNA can produce a repressor protein, while an activator may be produced from the same pre-mRNA in different conditions (Black D. L. 2000, Cell 103:367; Graveley, B. R. 2001, Trends Genet. 17:100). What is needed in the art are methods and compositions that can be used to regulate alternative splicing via riboswitches.

### BRIEF SUMMARY OF THE INVENTION

Disclosed herein is a regulatable gene expression construct comprising a nucleic acid molecule encoding an RNA comprising a riboswitch operably linked to a coding region, wherein the riboswitch regulates splicing of the RNA, wherein the riboswitch and coding region are heterologous, and wherein regulation of splicing affects processing of the RNA. The riboswitch can regulate alternative spicing of the RNA. The riboswitch can comprise an aptamer domain and an expression platform domain, wherein the aptamer domain and the expression platform domain are heterologous. The RNA can further comprise an intron. The riboswitch can be in the 3′ untranslated region of the RNA. The intron can be in the 3′ untranslated region of the RNA. An RNA processing site can be in the intron. Splicing of the intron can remove the RNA processing site from the RNA thereby affecting processing of the RNA. The affect on processing of the RNA can comprise elimination of processing of the RNA mediated by the RNA processing site. The affect on processing of the RNA can comprise an alteration in transcription termination. The affect on processing of the RNA can comprise an increase in degradation of the RNA. The affect on processing of the RNA can comprise an increase in turnover of the RNA. The riboswitch can overlap the 3′ splice junction of the intron. Splicing of the intron can reduce or eliminate the ability of the riboswitch to be activated. The splice junction can be a 5′ splice junction. The riboswitch can be in an intron of the RNA. RNA processing also can be regulated or affected independent of or without the involvement in splicing.

The expression platform domain can comprise a splice junction in the intron. The expression platform domain can comprise a splice junction at an end of the intron (that is, the 5′ splice junction or the 3′ splice junction). The RNA can further comprise an intron, wherein the expression platform domain comprises the branch site in the intron. The splice junction can be active when the riboswitch is activated. The splice junction can be active when the riboswitch is not activated. The riboswitch can be activated by a trigger molecule, such as thiamine pyrophosphate (TPP). The riboswitch can be a TPP-responsive riboswitch. The riboswitch can activate splicing. The riboswitch can repress splicing. The riboswitch can alter splicing of the RNA. The RNA can have a branched structure. The RNA can be pre-mRNA. The region of the aptamer with splicing control can be located, for example, in the P4 and P5 stem. The region of the aptamer with splicing control can also found, for example, in loop 5. The region of the aptamer with splicing control can also found, for example, in stem P2. Thus, for example, an expression platform domain can interact with the P4 and P5 sequences, the loop 5 sequence and/or the P2 sequences. Such aptamer sequences generally can be available for interaction with the expression platform domain only when a trigger molecule is not bound to the aptamer domain. The splice sites and/or branch sites can be located, for example, at positions between −130 to −160 relative to the 5′ end of the aptamer. The RNA can further comprise a second intron, wherein the 3′ splice site of the second intron is located at a position between −220 to −270 relative to the 5′ end of the aptamer domain.

Also disclosed is a method for affecting processing of RNA comprising introducing into the RNA a construct comprising a riboswitch, wherein the riboswitch is capable of regulating splicing of RNA, wherein the RNA comprises an intron, and wherein regulation of splicing affects processing of the RNA. The riboswitch can comprise an aptamer domain and an expression platform domain, wherein the aptamer domain and the expression platform domain are heterologous. The riboswitch can be in an intron of the RNA. The riboswitch can be activated by a trigger molecule, such as TPP. The riboswitch can be a TPP-responsive riboswitch. The riboswitch can activate splicing. The riboswitch can repress splicing. The riboswitch can alter splicing of the RNA. The splicing can occur non-naturally. The region of the aptamer with splicing control can be found, for example, in loop 5. The region of the aptamer with splicing control can also found, for example, in stem P2. The splice sites can be located, for example, at positions between −130 to −160 relative to the 5′ end of the aptamer. The construct can further comprise the intron.

Also disclosed is a method of affecting gene expression, the method comprising: bringing into contact (a) a cell comprising a construct comprising a nucleic acid molecule encoding an RNA comprising a riboswitch operably linked to a coding region, wherein the riboswitch regulates splicing of the RNA, wherein the riboswitch and coding region are heterologous, and wherein regulation of splicing affects processing of the RNA, and (b) an effective amount of a trigger molecule for the riboswitch, thereby affecting gene expression. The riboswitch can be a TPP-responsive riboswitch. The trigger molecule can be thiamin or TPP.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows that TPP aptamers are conserved and widespread in plant species. (A) Alignment of TPP aptamer sequences from various plant species reveals high conservation of sequence and structure. Nucleotides forming stems P1 through P5 are highlighted in different shadings and asterisks identify nucleotides that are conserved between all examples. Sequences are derived from A. thaliana (Ath, NC003071; SEQ ID NO:1), Brassica sativa (Bsa, EF588038; SEQ ID NO:2), Brassica oleracea (Bol, BH250462; SEQ ID NO:3), Boechera stricta (Bst, DU681973; SEQ ID NO:4), Carica papaya (Cpa, DX471004; SEQ ID NO:5), Citrus sinensis (Csi, DY305604; SEQ ID NO:6), Nicotiana tabacum (Nta, EF588039; SEQ ID NO:7), Nicotiana benthamiana (Nbe, EF588040; SEQ ID NO:8), Populus trichocarpa (Ptr, JGI, populus genome, LG_IX: 7897690-7897807; SEQ ID NO:9), Lotus japonicus (Lja, AG247551; SEQ ID NO:10), Lycopersicon esculentum (Les, EF588041; SEQ ID NO:11), Solanum tuberosum (Stu, DN941010; SEQ ID NO:12), Ocimum basilicum (Oba, EF588042; SEQ ID NO:13), Ipomoea nil (Ini, BJ566897; SEQ ID NO:14), Vitis vinifera (Vvi, AM442795; SEQ ID NO:15), Oryza sativa (Osa, NC008396; SEQ ID NO:16), Poa secunda (Pse, AF264021; SEQ ID NO:17), Triticum aestivum (Tae, CD879967; SEQ ID NO:18), Hordeum vulgare (Hvu, BM374959; SEQ ID NO:19), Sorghum bicolor (Sbi, CW250951; SEQ ID NO:20), Pinus taeda (Pta, CCGB, Contig116729 RTDS2_{—}8_E12.g1_A021: 551-686; SEQ ID NO:21), and Physcomitrella patens (Ppa, gnl|ti|856901678 (SEQ ID NO:22), gnl|ti|893553357 (SEQ ID NO:23), gnl|ti|876297717 (SEQ ID NO:24), (Lang et al., 2005)). The sequence for I. nil represents a splice variant derived from cDNA and is therefore lacking the 5′ end of the aptamer. The left P1 sequence for these sequences is GCACC except for the Ppa2 sequence, where it is GCGCC, and the Ini sequence. The right P1 sequence for these sequences is GUGUGC except for the Lja sequence, where it is GAGUGC, and the Les sequence, where it is GCGUGC. (B,C) Consensus sequences and secondary structure models of TPP riboswitch aptamers based on all representatives from plants (B; SEQ ID NOs:25 and 26) or bacterial and archaeal species (C; SEQ ID NOs:27-29) are similar. The mutual information reflects the probability for the occurrence of the boxed base pairs. The p-value is 0.1, 0.1, 0.01, 0.01, and 0.01 for the boxed base pairs in the P5 stem, from top to bottom. The p-value is 0.01, 0.01, and 0.1 for the boxed base pairs in the P4 stem, from top to bottom. The p-value is 0.01 for the boxed base pairs in the P1 stem and the P3a stem. The p-value is 0.1, 0.01, 0.01, 0.01, 0.01, and 0.01 for the boxed base pairs in the P3 stem, from left to right.

FIG. 2 shows that the architectures of THIC 3′ UTRs are conserved. (A) Organization of the 3′ region of THIC genes and derived transcript types are similar. The first box represents the last exon of the coding region with the stop codon UAA depicted. The stop codon is followed by an intron (except in L. esculentum, where the intron is located immediately in front of the stop codon), which is typically spliced in all transcript types (I, II, III). GU and AG notations identify 5′ and 3′ splice sites, respectively. Thick lines numbered 1 through 6 designate six regions of RNA transcripts whose lengths were analyzed as described in (B). Dashed lines indicate splicing events and the diamond symbol represents the transcript processing site. (B) Numbers of nucleotides in the regions defined in (A) are similar amongst seven plant species. The stacked bars for region 6 indicate the identification of transcripts of different lengths. (C) PCR amplification of THIC 3′ UTRs from cDNAs generated with polyT primer yields only type II RNAs in all species examined. RT-PCR products were separated using 1.5% agarose gel electrophoresis and visualized by ethidium bromide staining and UV illumination. “M” designates the marker lane containing DNAs of 100 base-pair increments. (D) RT-PCR analysis was conducted using the same cDNAs as used in (C) with primer combinations specific for 3′ UTRs of type I and III RNAs. (E) RT-PCR products of 3′ UTRs from type I and III RNAs from A. thaliana cDNAs generated with different RT primers. Primers used for RT were polyT, random hexamers or sequence specific primers that bind near the annotated end of THIC (221 nts downstream of the end of the aptamer) or further downstream (882 nts downstream of the end of the aptamer) as indicated. No RT indicates a control reaction using the RNA without reverse transcription as a template source.

FIG. 3 shows that THIC transcript types respond differently to changes in thiamin levels in A. thaliana. (A) qRT-PCR analysis was conducted on THIC transcripts from A. thaliana seedlings grown for 14 days on medium supplemented with 0, 0.1, and 1 mM thiamin. Total THIC transcripts and separately types I, II and III RNAs were detected using different primer combinations. cDNAs were generated using a polyT primer or random hexamers for detection of type I RNAs. Expression was normalized for each primer combination to the value measured using medium with no thiamin supplementation (open bars). Values are averages from three independent experiments and error bars represent standard deviation. (B) Northern blot analysis of THIC transcripts from the same samples described in (A). 20 μg total RNA was loaded per lane and analyzed using probes binding to the coding region of THIC, the extended 3′ UTR of types I and III RNAs, or the control transcript EIF4A1. The signal of THIC probes are shown in the size range between 2 and 3 kb. The 3′ UTR probe resulted in weak signals and exposure time was extended to 3 days compared to 1 day exposure for the other probes. (C) qRT-PCR analysis of the time-dependent effects of thiamin treatment on THIC transcripts from A. thaliana. Seedlings were grown for 14 days on thiamin free medium and subsequently sprayed with 50 μM thiamin and 0.25 mg ml⁻¹ Tween 80. Control seedlings were treated with a solution containing only Tween 80. Samples were collected after 4 h and 26 h and subjected to qRT-PCR analysis. Amounts of THIC transcripts were analyzed from cDNAs generated with polyT primer and normalized to the values of the control samples without application of thiamin (open bars). Values are averages from three independent experiments and error bars represent standard deviation. (D) Relative changes of the levels of THIC transcript types in wild-type (WT) and thiamin pyrophosphokinase double knockout (TPK-KO) A. thaliana plants. Seedlings were grown for 12 days on thiamin free medium and amounts of THIC transcript types were analyzed by qRT-PCR. Data were normalized to the values for the WT samples and reflect averages from three replicates, with error bars representing standard deviation.

FIG. 4 shows that the long 3′ UTR of THIC causes reduced gene expression independent of aptamer function. (A) Secondary structure model of the TPP aptamer generated after splicing in THIC type III RNA from A. thaliana (SEQ ID NOs: 30 and 31). Gray shaded nucleotides in stems P1 and P2 identify nucleobase changes compared to the original unspliced aptamer. Black boxed nucleotides were altered as shown to generate mutants M1 and M2 that do not bind TPP. (B) In-line probing analysis of TPP binding by the spliced aptamer depicted in (A). Lanes include RNAs loaded after no reaction (NR), after partial digestion with RNase T1 (T1), or after partial digestion with alkali (⁻OH). Sites 1 and 2 were quantified to establish the K_{D} as shown in (C). (C) Plot indicating the normalized fraction of RNA spontaneously cleaved versus the concentration of TPP for sites 1 and 2 in (B). (D) In vivo expression analysis of reporter constructs containing the 3′ UTR of A. thaliana type II or III RNAs fused to the 3′ end of the coding region of firefly luciferase (LUC). Constructs M1 and M2 are based on the 3′ UTR of type III RNAs, but contain the mutations shown in (A). LUC-III M1′ contains the inverted 3′ UTR sequence of construct LUC-III M1. Reporter constructs were analyzed in a transient Nicotiana benthamiana expression assay and values standardized to a coexpressed luciferase gene from Renilla. Expression was normalized to the fusion construct containing the 3′ UTR of type II RNA. Data shown are mean values of three independent experiments and the error bars represent standard deviation. (E) qRT-PCR analysis of EGFP reporter fusions that contain the 3′ UTRs of THIC type II or III RNAs from either A. thaliana (At) or N. benthamiana (Nb) after expression in a transient expression assay. Expression was standardized to a coexpressed DsRED reporter gene and normalized to the constructs containing a type II 3′ UTR. Data shown are mean values of two representative experiments and the error bars reflect standard deviation.

FIG. 5 shows in vivo analysis of riboswitch function. (A) Leaves from stably transformed A. thaliana lines expressing a reporter fusion of the complete 3′ region of AtTHIC fused to the 3′ end of EGFP were abscised and incubated with the petioles in water or in water supplemented with 0.02% thiamin. EGFP fluorescence was assessed at 0 h, 48 h, and 72 h after onset of treatment. One representative set of data from three repeats is shown, and the numbers identify different leaves from one transgenic line. (B) Quantitation of EGFP fluorescence of leaves depicted in (A) at three time points. The data represent average fluorescence intensity and standard deviation for each leaf. The plot also depicts average background fluorescence of WT leaves. (C) qRT-PCR analysis of total EGFP and THIC transcripts from leaves incubated for 72 h in water or 0.02% thiamin. Transcript amounts were standardized to an internal reference transcript and normalized to transcript abundance in water treated samples. Values are averages from four independent experiments using different transgenic lines and error bars represent standard deviation. (D,E) RT-PCR analysis of different 3′ UTRs of EGFP and THIC transcript types from A. thaliana reporter transformants grown in the absence of exogenous thiamin. For cDNA generation, a polyT primer, random hexamers or two different gene specific primers (binding either 221 or 882 nts downstream of the end of the aptamer) were used as indicated. The forward primers were specific for the end of the last exon of the coding region of EGFP (left) or THIC (right), whereas the reverse primer was either a polyT primer (D) or homologous to a region 221 nts downstream of the end of the aptamer (E). RT-PCR products were separated and visualized as described in the description of FIG. 2. M designates the marker lanes containing DNAs of 100 base-pair increments. No RT indicates a control reaction using the RNA without reverse transcription as a template source. I-1 and I-2 represent type I RNAs with the upstream intron following the stop codon unspliced or spliced, respectively. The lowest band in the polyT reaction in (E) results from amplification of THIC type II RNAs with polyT primer remaining from the RT reaction. Additional unmarked bands correspond to nonspecific amplification as confirmed by cloning and sequencing of all RT-PCR products.

FIG. 6 shows the effects of aptamer mutations on riboswitch function. (A) Secondary structure model and sequence of the WT TPP aptamer from A. thaliana genomic sequence and located in the 3′ region of THIC that was fused to EGFP (SEQ ID NOs: 32 and 33). Black boxed nucleotides were altered as indicated to generate mutants M2, M3 and M4 with impaired TPP binding. (B) Quantitation of EGFP fluorescence in leaves from A. thaliana transformants expressing reporter constructs containing the WT aptamer sequence or mutated versions M2, M3 and M4. Leaves were excised and incubated with their petioles in water or 0.02% thiamin for 72 h before fluorescence analysis. Values are averages from at least three independent experiments using different transgenic lines. Error bars represent standard deviation. (C) qRT-PCR analyses of EGFP and THIC transcript amounts in A. thaliana transformants described in (B). Transcript amounts (standardized using a reference transcript) were normalized to transcript abundance in water treated samples. Values are averages from two to four independent experiments using different transgenic lines. Error bars represent standard deviation. (D,E) RT-PCR analyses of 3′ UTRs of EGFP and THIC transcripts from A. thaliana transformants with mutations M2 or M3. RT-PCR analyses were performed as described in the description of FIGS. 5D and 5E. Forward primers were homologous to the end of the last exon of the coding region of EGFP or THIC, and the reverse primer was a polyT primer (D) or complementary to a region 221 nts downstream of the end of the aptamer (E). Kbp designates kilobase pairs.

FIG. 7 shows the mechanism of riboswitch function in plants. (A) TPP causes changes in RNA structure near to the 5′ splice site, which is important for the formation of THIC type III RNA. For in-line probing, a 5′ ³²P-labelled RNA starting 14 nts upstream of the 5′ splice site (+1) and expanding to the 3′ end of the TPP aptamer (nucleotides-14-261) from A. thaliana was incubated in the absence (−) or presence (+) of 10 μM TPP and the resulting spontaneous cleavage products were separated by polyacrylamide gel electrophoresis. Markers are RNAs partially digested with RNase T1 (T1) or alkali (⁻OH). The graph depicts the relative band intensities in the lanes indicated. (B) Base-pairing potential between the 5′ splice site region and the P4-P5 stems of the TPP aptamer (SEQ ID NOs:34-47; complementary nucleotides are shaded). Stretches of complementary nucleotides are also present in all other plant THIC mRNA sequences available. (C) A model for THIC TPP riboswitch function in plants includes control of splicing and alternative 3′ end processing of transcripts. When TPP concentrations are low (left), portions of stems P4 and P5 interact with the 5′ splice site and thereby prevent splicing. The transcript processing site located between the 5′ splice site and the TPP aptamer is retained, and its use results in formation of transcripts with short 3′ UTRs that permit high expression. In the presence of elevated TPP concentrations (right), TPP binds to the aptamer cotranscriptionally, which leads to a structural change that prevents interaction with the 5′ splice site. Splicing occurs and removes the transcript processing site. Transcription continues and alternative processing sites in the extended 3′ UTR give rise to THIC type III RNAs. The long 3′ UTRs lead to increased RNA degradation, causing reduced expression of THIC.

FIG. 8 shows genomic DNA sequence contexts of TPP riboswitches in THIC genes from different plant species (SEQ ID NOs:48-54). [symbol] identifies the stop codon of the THIC open reading frame; [symbol] and [symbol] designate 5′ and 3′ splice sites of the first intron (shown in italics). [symbol] and [symbol] identify the splice sites used for generation of type III RNAs. The 3′ UTR of type II RNAs is underlined, the aptamer sequence is in bold underline. The displayed 3′ ends of the sequences correspond to the gene annotations for Arabidopsis thaliana and Oryza sativa. For the other plant species the displayed sequences comply with 3′ ends identified by RT-PCR.

FIG. 9 shows that the THIC promoter from A. thaliana is not responsible for down regulation of THIC expression after thiamin supplementation. A construct consisting of a 1595 by fragment of the THIC promoter from A. thaliana was fused to the reporter gene β-glucuronidase (GUS) and transformed into A. thaliana. Amounts of GUS and THIC transcripts were analyzed by qRT-PCR and normalized to the expression of the reference transcript eEF-1α in 9 day old seedlings grown on medium without thiamin or supplemented with 100 μM thiamin. Data are mean values from three different transgenic lines and from three independent experiments. Error bars represent standard deviation.

FIG. 10 shows circadian expression of THIC. (A) qRT-PCR analysis of total THIC transcripts from plants incubated for 48 h under continuous light. Plants were grown for 11 days in light/dark cycles (16/8 h) on medium without thiamin or medium supplemented with 100 μM thiamin. On the morning of the 12^{th} day, plants were transferred to continuous light and samples were taken every 3 hours. Expression was normalized to the value of the sample at time point 0 from plants grown on thiamin free medium. Error bars represent standard deviation of triplicate qRT-PCR analyses. The absence of error bars indicates they are smaller than the diameter of the data points. (B) qRT-PCR analysis of THIC type III RNAs. Plant material and data normalization are as described for (A).

FIG. 11 shows the effect of 3′ UTRs from different types of THIC transcripts on reporter gene expression. (A) Reporter fusion constructs consisting of EGFP and the 3′ UTRs from THIC-II or RNAs from A. thaliana were expressed using a transient leaf infiltration assay and fluorescence was measured after 48 h and 96 h. Results were comparable to those observed with the luciferase reporter constructs. It is known that transient expression systems can lead to post-transcriptional gene silencing (PTGS) (Johansen and Carrington, 2001; Voinnet et al., 2003). To assess the possible effects of PTGS, the relative expression of the two 3′ UTR variants was determined in the absence or presence of P19, a known suppressor of gene silencing. Fluorescence was normalized relative to the value for the construct containing the 3′ UTR of THIC-II. Data are averages from four independent experiments and error bars represent standard deviation. The ratio of the activity for the two constructs remained unchanged after coexpression of P19, indicating that PTGS in not involved in the observed differences. (B) Relative fluorescence of EGFP reporter constructs containing the 3′ UTRs from N. benthamiana THIC type II and III RNAs after expression in a leaf infiltration assay. Expression was normalized relative to the value for the construct containing the 3′ UTR of THIC type II RNAs. Values are averages from two independent experiments and error bars represent standard deviation. The results are equivalent to those observed with constructs based on the 3′ UTRs from A. thaliana.

FIG. 12 shows TPP induced modulation in the 5′ flanking sequence of the aptamer. An RNA starting 14 nts upstream of the 5′ splice site and extending to the end of the aptamer (−14-261) was produced by in vitro transcription and 5′ end labeled with ³²P. After performing in-line probing reactions in the absence or presence of 10 μM TPP, cleavage products were separated by page. Markers were generated by RNase T1 treatment (T1) or partial alkaline digestion (⁻OH). The G residue of the 5′ splice site was defined as position 1 and the aptamer SPANS nts 146-256. TPP dependent modulation outside of the aptamer is mainly observed in the region next to the 5′ splice site. However, additional structural changes reveal that ligand dependent modulation elsewhere in the 5′ flank might be important for control of the 5′ splice site structure.

### DETAILED DESCRIPTION OF THE INVENTION

The disclosed methods and compositions can be understood more readily by reference to the following detailed description of particular embodiments and the Examples included therein and to the Figures and their previous and following description.

Messenger RNAs are typically thought of as passive carriers of genetic information that are acted upon by protein- or small RNA-regulatory factors and by ribosomes during the process of translation. It was discovered that certain mRNAs carry natural aptamer domains and that binding of specific metabolites directly to these RNA domains leads to modulation of gene expression. Natural riboswitches exhibit two surprising functions that are not typically associated with natural RNAs. First, the mRNA element can adopt distinct structural states wherein one structure serves as a precise binding pocket for its target metabolite. Second, the metabolite-induced allosteric interconversion between structural states causes a change in the level of gene expression by one of several distinct mechanisms. Riboswitches typically can be dissected into two separate domains: one that selectively binds the target (aptamer domain) and another that influences genetic control (expression platform). It is the dynamic interplay between these two domains that results in metabolite-dependent allosteric control of gene expression.

Distinct classes of riboswitches have been identified and are shown to selectively recognize activating compounds (referred to herein as trigger molecules). For example, coenzyme B₁₂, glycine, thiamine pyrophosphate (TPP), and flavin mononucleotide (FMN) activate riboswitches present in genes encoding key enzymes in metabolic or transport pathways of these compounds. The aptamer domain of each riboswitch class conforms to a highly conserved consensus sequence and structure. Thus, sequence homology searches can be used to identify related riboswitch domains. Riboswitch domains have been discovered in various organisms from bacteria, archaea, and eukarya.

More than a dozen structural classes of riboswitches have been reported in eubacteria that sense 10 different metabolites (Mandal 2004; Winkler 2005; Breaker 2006; Fuchs 2006; Roth). A eubacterial riboswitch selective for the queuosine precursor preQ₁ contains an unusually small aptamer domain. Nat. Struct. Mol. Biol. (2007), and numerous other classes are currently being characterized. The aptamer domain of each riboswitch is distinguished by its nucleotide sequence (Rodionov 2002; Vitreschak 2002; Vitreschak 2003) and folded structure (Nahvi 2004; Batey 2004; Serganov 2004; Montange 2006; Thore 2006; Serganov 2006; Edwards 2006) which remain highly conserved even between distantly related organisms. Riboswitches usually include an expression platform that modulates gene expression in response to metabolite binding by the aptamer, although expression platforms can differ extensively in sequence, structure, and control mechanism.

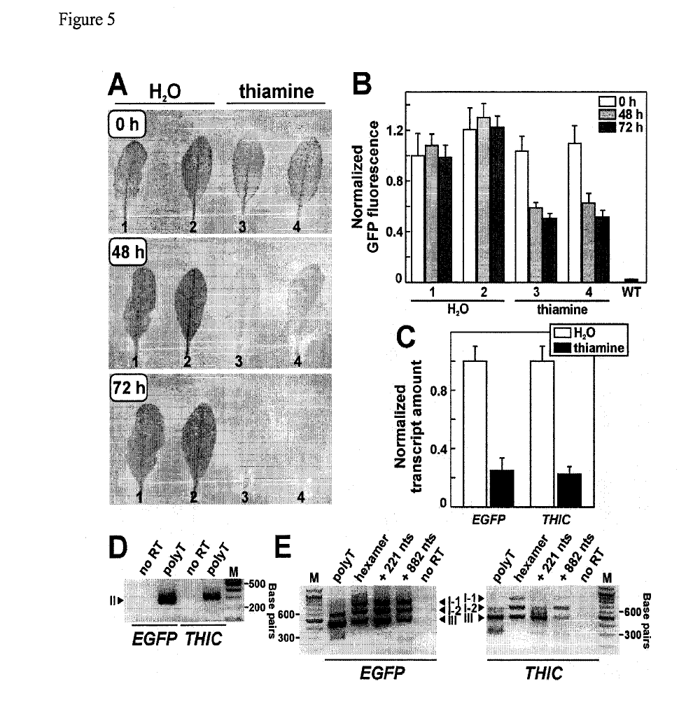

The exceptional level of aptamer conservation enables the use of bioinformatics to identify similar riboswitch representatives in diverse organisms. Currently, only sequences that conform to the TPP riboswitch aptamer consensus have been identified in organisms from all three domains of life (Sudarsan 2003). Although some predicted eukaryotic TPP aptamers from fungi (Sudarsan 2003; Galagan 2005) (FIG. 5) and plants were shown to bind TPP (Sudarsan 2003 Yamauchi), the precise mechanisms by which metabolite binding controls gene expression were previously unknown. In fungi, each TPP aptamer resides within an intron in the 5′ untranslated region (UTR) or the protein coding region of an mRNA, implying that mRNA splicing is controlled by metabolite binding (Sudarsan 2003; Kubodera 2003). In plants, each TPP aptamer resides within the 3′ untranslated region (UTR) or the protein coding region of an mRNA. It has been discovered that plant TPP-responsive riboswitches affect processing of the RNA in which they reside.

### A. General Organization of Riboswitch RNAs

Bacterial riboswitch RNAs are genetic control elements that are located primarily within the 5′-untranslated region (5′-UTR) of the main coding region of a particular mRNA. Structural probing studies (discussed further below) reveal that riboswitch elements are generally composed of two domains: a natural aptamer (T. Hermann, D. J. Patel, Science 2000, 287, 820; L. Gold, et al., Annual Review of Biochemistry 1995, 64, 763) that serves as the ligand-binding domain, and an ‘expression platform’ that interfaces with RNA elements that are involved in gene expression (e.g. Shine-Dalgarno (SD) elements; transcription terminator stems). These conclusions are drawn from the observation that aptamer domains synthesized in vitro bind the appropriate ligand in the absence of the expression platform (see Examples 2, 3 and 6 of U.S. Application Publication No. 2005-0053951). Moreover, structural probing investigations suggest that the aptamer domain of most riboswitches adopts a particular secondary- and tertiary-structure fold when examined independently, that is essentially identical to the aptamer structure when examined in the context of the entire 5′ leader RNA. This indicates that, in many cases, the aptamer domain is a modular unit that folds independently of the expression platform (see Examples 2, 3 and 6 of U.S. Application Publication No. 2005-0053951).

Ultimately, the ligand-bound or unbound status of the aptamer domain is interpreted through the expression platform, which is responsible for exerting an influence upon gene expression. The view of a riboswitch as a modular element is further supported by the fact that aptamer domains are highly conserved amongst various organisms (and even between kingdoms as is observed for the TPP riboswitch) (N. Sudarsan, et al., RNA 2003, 9, 644), whereas the expression platform varies in sequence, structure, and in the mechanism by which expression of the appended open reading frame is controlled. For example, ligand binding to the TPP riboswitch of the tenA mRNA of B. subtilis causes transcription termination (A. S. Mironov, et al., Cell 2002, 111, 747). This expression platform is distinct in sequence and structure compared to the expression platform of the TPP riboswitch in the thiM mRNA from E. coli, wherein TPP binding causes inhibition of translation by a SD blocking mechanism (see Example 2 of U.S. Application Publication No. 2005-0053951). The TPP aptamer domain is easily recognizable and of near identical functional character between these two transcriptional units, but the genetic control mechanisms and the expression platforms that carry them out are very different.

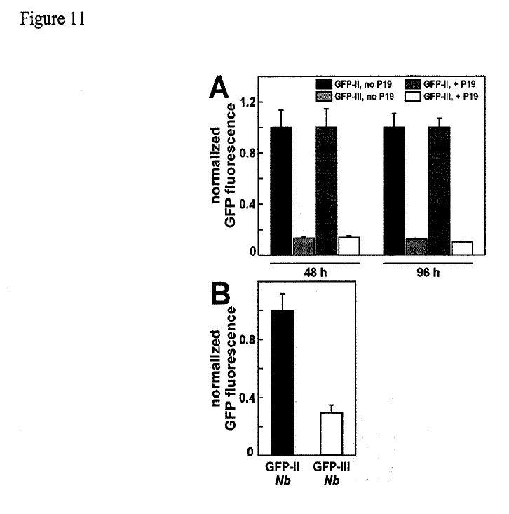

Aptamer domains for riboswitch RNAs typically range from ˜70 to 170 nt in length (FIG. 11 of U.S. Application Publication No. 2005-0053951). This observation was somewhat unexpected given that in vitro evolution experiments identified a wide variety of small molecule-binding aptamers, which are considerably shorter in length and structural intricacy (T. Hermann, D. J. Patel, Science 2000, 287, 820; L. Gold, et al., Annual Review of Biochemistry 1995, 64, 763; M. Famulok, Current Opinion in Structural Biology 1999, 9, 324). Although the reasons for the substantial increase in complexity and information content of the natural aptamer sequences relative to artificial aptamers remains to be proven, this complexity is believed required to form RNA receptors that function with high affinity and selectivity. Apparent K_{D} values for the ligand-riboswitch complexes range from low nanomolar to low micromolar. It is also worth noting that some aptamer domains, when isolated from the appended expression platform, exhibit improved affinity for the target ligand over that of the intact riboswitch. (˜10 to 100-fold) (see Example 2 of U.S. Application Publication No. 2005-0053951). Presumably, there is an energetic cost in sampling the multiple distinct RNA conformations required by a fully intact riboswitch RNA, which is reflected by a loss in ligand affinity. Since the aptamer domain must serve as a molecular switch, this might also add to the functional demands on natural aptamers that might help rationalize their more sophisticated structures.

### B. The TPP Riboswitch

The coenzyme thiamine pyrophosphate (TPP) is an active form of vitamin B1, an essential participant in many protein-catalyzed reactions. Organisms from all three domains of life, including bacteria, plants and fungi, use TPP-sensing riboswitches to control genes responsible for importing or synthesizing thiamine and its phosphorylated derivatives, making this riboswitch class the most widely distributed member of the metabolite-sensing RNA regulatory system. The structure reveals a folded RNA in which one subdomain forms an intercalation pocket for the 4-amino-5-hydroxymethyl-2-methylpyrimidine moiety of TPP, whereas another subdomain forms a wider pocket that uses bivalent metal ions and water molecules to make bridging contacts to the pyrophosphate moiety of the ligand. The two pockets are positioned to function as a molecular measuring device that recognizes TPP in an extended conformation. The central thiazole moiety is not recognized by the RNA, which explains why the antimicrobial compound pyrithiamine pyrophosphate targets this riboswitch and downregulates the expression of thiamine metabolic genes. Both the natural ligand and its drug-like analogue stabilize secondary and tertiary structure elements that are harnessed by the riboswitch to modulate the synthesis of the proteins coded by the mRNA. In addition, this structure provides insight into how folded RNAs can form precision binding pockets that rival those formed by protein genetic factors.

Three TPP riboswitches were examined in the filamentous fungus Neurospora crassa, and it was found that one activates and two repress gene expression by controlling mRNA splicing (Cheah 2007). A detailed mechanism involving riboswitch-mediated base-pairing changes and alternative splicing control was elucidated for precursor NMT1 mRNAs, which code for a protein involved in TPP metabolism (Cheah 2007). These results demonstrate that eukaryotic cells employ metabolite-binding RNAs to regulate RNA splicing events important for the control of key biochemical processes.

It was discovered that TPP riboswitches are present in the 3′ untranslated region (UTR) of the thiamin biosynthetic gene THIC of all plant species examined. The THIC TPP riboswitch controls the formation of transcripts with alternative 3′ UTR lengths, which affect mRNA stability and protein production. It has been demonstrated that riboswitch-mediated regulation of alternative 3′ end processing is critical for TPP-dependent feedback control of THIC expression. The data reveal a mechanism whereby metabolite-dependent alteration of RNA folding controls splicing and alternative 3′ end processing of mRNAs.

TPP riboswitches are present in a variety of plant species where they reside in the 3′ UTR of the thiamin metabolic gene THIC. Formation of THIC transcripts with alternative 3′ UTR lengths is dependent on riboswitch function and mediates feedback regulation of THIC expression in response to changes in cellular TPP levels. The data indicate that 3′ UTR length correlates with transcript stability, thereby establishing a basis for gene control by alternative 3′ end processing. A detailed mechanism for TPP riboswitch function in plants is presented (Example 1), which includes aptamer mediated control of splicing and differential 3′ end processing of THIC mRNAs.

The presence of highly conserved TPP-binding aptamers in the 3′ UTRs of the THIC genes from the plant species Arabidopsis thaliana, Oryza sativa and Poa secunda had been reported previously (Sudarsan et al., 2003). The collection of plant TPP aptamer representatives was expanded by sequencing THIC genes from additional plant species and by conducting database searches for nucleotide sequences that conform to the TPP aptamer consensus. After cDNA sequences were obtained, the corresponding regions from genomic DNAs of each species were cloned and sequenced (see Experimental Procedures for details), thus providing the sequences of both the initial and the processed mRNA molecules.

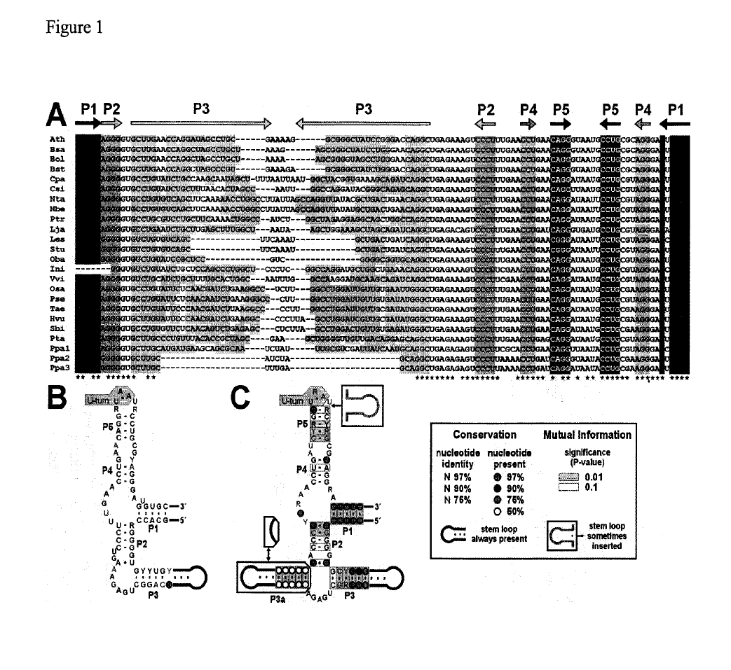

An alignment of all available TPP aptamer sequences from plants reveals a high level of conservation of nucleotide sequence and a secondary structure consisting of stems P1 through P5 (FIG. 1A). The major differences between eukaryotic TPP riboswitch aptamers from plants (FIG. 1B) and filamentous fungi (Cheah et al., 2007) compared to their bacterial and archaeal counterparts (FIG. 1C) (Winkler et al., 2002; Rodionov et al. 2002) are the consistent absence of a P3a stem frequently present in bacterial representatives and the variable length of the P3 stem in eukaryotes. Neither region is involved in TPP binding (Edwards and Ferre-D'Amare, 2006; Serganov et al., 2006; Thore et al., 2006; Cheah et al., 2007) and therefore these differences should not affect ligand binding specificity.

The TPP aptamer is found in the 3′ UTR of all known THIC examples from monocots, dicots and the conifer Pinus taeda. Interestingly, in the moss Physcomitrella patens, the TPP aptamer is present in the 3′ UTR of THIC (Ppa1), and also resides in the 3′ region of two genes that are homologous to the thiamin biosynthetic gene THI4 (Ppa2, Ppa3). This latter observation, and the observation that fungi also have TPP aptamers associated with multiple different genes (Cheah et al., 2007), indicates that eukaryotes likely use variants of the same riboswitch class to control multiple genes in response to changing concentrations of a key metabolite.

A striking characteristic of TPP aptamers from plants is the high level of nucleotide sequence conservation. Approximately 80% of the nucleotides (excluding the P3 stem) are conserved in all plant examples. In contrast, less than 40% are conserved in filamentous fungi. Most differences among plant TPP aptamers are found in the P3 stem, which varies both in length and sequence. Also, the length of the P3 stem varies between TPP aptamer representatives in the same species, as is observed in P. patens (FIG. 1A). The presence of both an extended P3 stem in THIC and very short P3 stems in THI4 indicates that there is no species-specific requirement for this component of the aptamer.

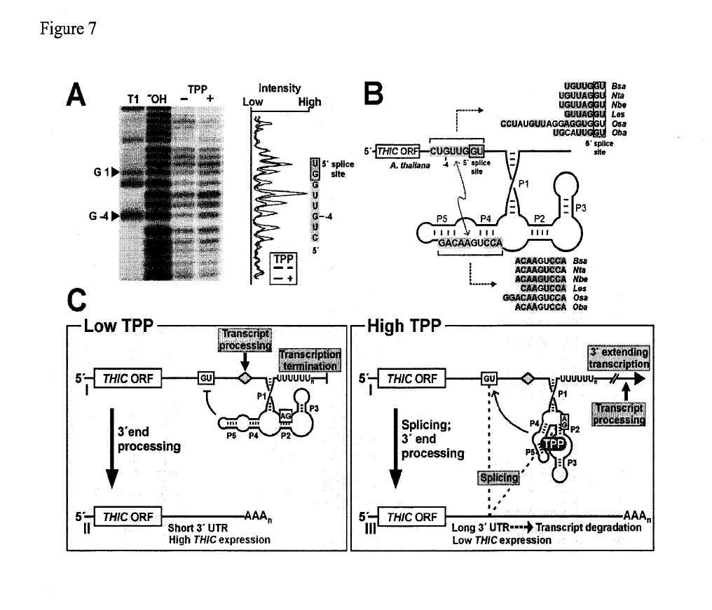

TPP riboswitch regulation in plants involves the metabolite-mediated control of splicing and alternative 3′ end processing of mRNA transcripts (FIG. 7C). When TPP concentration in cells is low, the aptamer interacts with the 5′ splice site and prevents splicing. This intron carries a major processing site that permits transcript cleavage and polyadenylation. Processing from this site produces THIC-II transcripts that carry short 3′ UTRs and that yield high expression of the THIC gene.

When TPP concentrations are high, TPP binding to the aptamer prevents pairing to the 5′ splice site. As a result, the 5′ splice site becomes accessible and is used in a splicing event that removes the major processing site. Transcription subsequently extends up to 1 kb and the use of processing sites located downstream gives rise to THIC-III RNAs that carry much longer 3′ UTRs. The long 3′ UTRs cause increased transcript degradation and THIC expression is reduced. Previous studies have shown that extended transcription occurs in the absence of transcript processing, thus revealing the interconnectivity of these processes (Buratowski, 2005; Proudfoot, 2004; Proudfoot et al., 2002).

TPP riboswitches are also described in U.S. Patent Application Publication No. US-2005-0053951, which is incorporated herein in its entirety and also in particular is incorporated by reference for its description of TTP riboswitch structure, function and use. It is specifically contemplated that any of the subject matter and description of U.S. Patent Application Publication No. US-2005-0053951, and in particular any description of TTP riboswitch structure, function and use in U.S. Patent Application Publication No. US-2005-0053951 can be specifically included or excluded from the other subject matter disclosed herein.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference to each of various individual and collective combinations and permutation of these compounds can not be explicitly disclosed, each is specifically contemplated and described herein. For example, if a riboswitch or aptamer domain is disclosed and discussed and a number of modifications that can be made to a number of molecules including the riboswitch or aptamer domain are discussed, each and every combination and permutation of riboswitch or aptamer domain and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Riboswitches

Riboswitches are expression control elements that are part of an RNA molecule to be expressed and that change state when bound by a trigger molecule. Riboswitches typically can be dissected into two separate domains: one that selectively binds the target (aptamer domain) and another that influences genetic control (expression platform domain). It is the dynamic interplay between these two domains that results in metabolite-dependent allosteric control of gene expression. Disclosed are isolated and recombinant riboswitches, recombinant constructs containing such riboswitches, heterologous sequences operably linked to such riboswitches, and cells and transgenic organisms harboring such riboswitches, riboswitch recombinant constructs, and riboswitches operably linked to heterologous sequences. The heterologous sequences can be, for example, sequences encoding proteins or peptides of interest, including reporter proteins or peptides. Preferred riboswitches are, or are derived from, naturally occurring riboswitches. For example, the aptamer domain can be, or be derived from, the aptamer domain of a naturally occurring riboswitch. The riboswitch can include or, optionally, exclude, artificial aptamers. For example, artificial aptamers include aptamers that are designed or selected via in vitro evolution and/or in vitro selection. The riboswitches can comprise the consensus sequence of naturally occurring riboswitches. Consensus sequences for a variety of riboswitches are described in U.S. Application Publication No. 2005-0053951, such as in FIG. 11. The consensus sequence of plant TPP-responsive riboswitches is shown in FIG. 1B and specific examples are shown in FIG. 1A.

Disclosed herein is a regulatable gene expression construct comprising a nucleic acid molecule encoding an RNA comprising a riboswitch operably linked to a coding region, wherein the riboswitch regulates splicing of the RNA, wherein the riboswitch and coding region are heterologous, and wherein regulation of splicing affects processing of the RNA. The riboswitch can regulate alternative spicing of the RNA. The riboswitch can comprise an aptamer domain and an expression platform domain, wherein the aptamer domain and the expression platform domain are heterologous. The RNA can further comprise an intron. The riboswitch can be in the 3′ untranslated region of the RNA. The intron can be in the 3′ untranslated region of the RNA. An RNA processing site can be in the intron. Splicing of the intron can remove the RNA processing site from the RNA thereby affecting processing of the RNA. The affect on processing of the RNA can comprise elimination of processing of the RNA mediated by the RNA processing site. The affect on processing of the RNA can comprise an alteration in transcription termination. The affect on processing of the RNA can comprise an increase in degradation of the RNA. The affect on processing of the RNA can comprise an increase in turnover of the RNA. The riboswitch can overlap the 3′ splice junction of the intron. Splicing of the intron can reduce or eliminate the ability of the riboswitch to be activated. The splice junction can be a 5′ splice junction. The riboswitch can be in an intron of the RNA. RNA processing also can be regulated or affected independent of or without the involvement in splicing.

The expression platform domain can comprise a splice junction in the intron. The expression platform domain can comprise a splice junction at an end of the intron (that is, the 5′ splice junction or the 3′ splice junction). The RNA can further comprise an intron, wherein the expression platform domain comprises the branch site in the intron. The splice junction can be active when the riboswitch is activated. The splice junction can be active when the riboswitch is not activated. The riboswitch can be activated by a trigger molecule, such as thiamine pyrophosphate (TPP). The riboswitch can be a TPP-responsive riboswitch. The riboswitch can activate splicing. The riboswitch can repress splicing. The riboswitch can alter splicing of the RNA. The RNA can have a branched structure. The RNA can be pre-mRNA. The region of the aptamer with splicing control can be located, for example, in the P4 and P5 stem. The region of the aptamer with splicing control can also found, for example, in loop 5. The region of the aptamer with splicing control can also found, for example, in stem P2. Thus, for example, an expression platform domain can interact with the P4 and P5 sequences, the loop 5 sequence and/or the P2 sequences. Such aptamer sequences generally can be available for interaction with the expression platform domain only when a trigger molecule is not bound to the aptamer domain. The splice sites and/or branch sites can be located, for example, at positions between −130 to −160 relative to the 5′ end of the aptamer. The RNA can further comprise a second intron, wherein the 3′ splice site of the second intron is located at a position between −220 to −270 relative to the 5′ end of the aptamer domain.

Also disclosed is a method for affecting processing of RNA comprising introducing into the RNA a construct comprising a riboswitch, wherein the riboswitch is capable of regulating splicing of RNA, wherein the RNA comprises an intron, and wherein regulation of splicing affects processing of the RNA. The riboswitch can comprise an aptamer domain and an expression platform domain, wherein the aptamer domain and the expression platform domain are heterologous. The riboswitch can be in an intron of the RNA. The riboswitch can be activated by a trigger molecule, such as TPP. The riboswitch can be a TPP-responsive riboswitch. The riboswitch can activate splicing. The riboswitch can repress splicing. The riboswitch can alter splicing of the RNA. The splicing can occur non-naturally. The region of the aptamer with splicing control can be found, for example, in loop 5. The region of the aptamer with splicing control can also found, for example, in stem P2. The splice sites can be located, for example, at positions between −130 to −160 relative to the 5′ end of the aptamer. The construct can further comprise the intron.

Also disclosed is a method of affecting gene expression, the method comprising: bringing into contact (a) a cell comprising a construct comprising a nucleic acid molecule encoding an RNA comprising a riboswitch operably linked to a coding region, wherein the riboswitch regulates splicing of the RNA, wherein the riboswitch and coding region are heterologous, and wherein regulation of splicing affects processing of the RNA, and (b) an effective amount of a trigger molecule for the riboswitch, thereby affecting gene expression. The riboswitch can be a TPP-responsive riboswitch. The trigger molecule can be thiamin or TPP.

The riboswitch can alter splicing of the RNA. For example, activation of the riboswitch can allow or promote splicing, allow or promote alternative splicing, prevent or reduce splicing or the predominate splicing, prevent or reduce alternative splicing, or allow or promote splicing or the predominate splicing. As other examples, a deactivated riboswitch or deactivation of the riboswitch can allow or promote alternative splicing, prevent or reduce splicing or the predominate splicing, prevent or reduce alternative splicing, or allow or promote splicing or the predominate splicing. Generally, the form of splicing regulation can be determined by the physical relationship of the riboswitch to the splice junctions, alternative splice junctions and branch sites in the RNA molecule. For example, activation/deactivation of riboswitches generally involves formation and/or disruption of alternative secondary structures (for example, base paired stems) in RNA and this change in structure can be used to hide or expose functional RNA sequences. The expression platform domain of a riboswitch generally comprises such functional RNA sequences. Thus, for example, by including a slice junction or a branch site in the expression platform domain of a riboswitch in such a way that the spice junction or branch site is alternately hidden or exposed as the riboswitch is activated or deactivated, or vice versa, splicing of the RNA can be regulated or affected.

Regulation of splicing can affect processing of the RNA in which splicing is regulated. For example, an intron in the RNA can include an RNA processing signal or site. Splicing of the RNA can result in elimination of the processing signal or site. For example, a transcription termination signal or RNA cleavage site in the 3′ UTR of a mRNA can be deleted from the RNA if it resides in an intron that is spliced out of the RNA. Regulation of the splicing of that intron by a riboswitch as described herein can thus affect the processing of the RNA. As another example, an RNA processing signal or site can be created via splicing of an intron or different elements of an RNA processing system, signal or site can be brought into or taken out of an operable arrangement by splicing of an intron. As another example, an RNA processing signal or site can be brought into or taken out of an operable proximity with other elements of the RNA.

RNA processing can also be affected directly by a riboswitch without mediation by regulation of splicing. For example, an RNA processing signal or site can be in the expression platform domain of a riboswitch. In this way, the alteration in the structural relationship of the expression platform (and thus of the RNA processing signal or site) by activation of the riboswitch can affect processing by affecting the ability of the RNA processing signal or site to operate.

The riboswitch can affect RNA processing. By “affect RNA processing” is meant that the riboswitch can either directly or indirectly (via regulation of splicing, for example) act upon RNA to allow, stimulate, reduce or prevent RNA processing to take place. This can include, for example, allowing any processing to take place. This can increase or decrease processing by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% or more compared to the number of processing events that would have taken place without the riboswitch.

RNA processing can include, for example, transcription termination, formation of the 3′ terminus of the RNA, polyadenylation, and degradation or turnover of the RNA. As used herein, and RNA processing signal or site is a sequence, structure or location in an RNA that mediates, signals or is required for an RNA processing event or condition. For example, certain sequences or structures can signal transcription termination, RNA cleavage or polyadenylation.

The riboswitch can activate or repress splicing. By “activate splicing” is meant that the riboswitch can either directly or indirectly act upon RNA to allow splicing to take place. This can include, for example, allowing any splicing to take place (such as a single splice versus no splice) or allowing alternative splicing to take place. This can increase splicing by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% or more compared to the number of splicing events that would have taken place without the riboswitch.

By “repress splicing” is meant that the riboswitch can either directly or indirectly act upon RNA to suppress splicing. This can include, for example, preventing any splicing or reducing splicing from taking place (such as no splice versus a single splice) or preventing or reducing alternative splicing from taking place. This can decrease alternative splicing by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% compared to the number of alternative splicing events that would have taken place without the riboswitch.

The riboswitch can activate or repress alternative splicing. By “activate alternative splicing” is meant that the riboswitch can either directly or indirectly act upon RNA to allow alternative splicing to take place. This can increase alternative splicing by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% or more compared to the number of alternative splicing events that would have taken place without the riboswitch.

By “repress alternative splicing” is meant that the riboswitch can either directly or indirectly act upon RNA to suppress alternative splicing. This can decrease alternative splicing by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% compared to the number of alternative splicing events that would have taken place without the riboswitch.

The riboswitch can affect expression of a protein encoded by the RNA. For example, regulation of splicing or alternative splicing can affect the ability of the RNA to be translated, alter the coding region, or alter the translation initiation or termination. Alternative splicing can, for example, cause a start or stop codon (or both) to appear in the processed transcript that is not present in normally processed transcripts. As another example, alternative splicing can cause the normal start or stop codon to be removed from the processed transcript. A useful mode for using riboswitch-regulated splicing to regulate expression of a protein encoded by an RNA is to introduce a riboswitch in an intron in the 5′ untranslated region of the RNA and include or make use of a start codon in the intron such that the start codon in the intron will be the first start codon in the alternatively spliced RNA. Another useful mode for using riboswitch-regulated splicing to regulate expression of a protein encoded by an RNA is to introduce a riboswitch in an intron in the 5′ untranslated region of the RNA and include or make use of a short open reading frame in the intron such that the reading frame will appear first in the alternatively spliced RNA.

The RNA molecule can have a branched structure. For example, in the fungal TPP riboswitch (Cheah 2007), when TPP concentration is low, the newly transcribed mRNA adopts a structure that occludes the second 5′ splice site, while leaving the branch site available for splicing. Pre-mRNA splicing from the first 5′ splice site leads to production of the 1-3 form of mRNA and expression of the NMT1 protein. When TPP concentration is high, ligand binding to the TPP aptamer causes allosteric changes in RNA folding to increase the structural flexibility near the second 5 splice site and to occlude nucleotides near the branch site.

The disclosed riboswitches, including the derivatives and recombinant forms thereof, generally can be from any source, including naturally occurring riboswitches and riboswitches designed de novo. Any such riboswitches, as long as they have been determined to regulate alternative splicing, can be used in or with the disclosed methods. However, different types of riboswitches can be defined and some such sub-types can be useful in or with particular methods (generally as described elsewhere herein). Types of riboswitches include, for example, naturally occurring riboswitches, derivatives and modified forms of naturally occurring riboswitches, chimeric riboswitches, and recombinant riboswitches. A naturally occurring riboswitch is a riboswitch having the sequence of a riboswitch as found in nature. Such a naturally occurring riboswitch can be an isolated or recombinant form of the naturally occurring riboswitch as it occurs in nature. That is, the riboswitch has the same primary structure but has been isolated or engineered in a new genetic or nucleic acid context. Chimeric riboswitches can be made up of, for example, part of a riboswitch of any or of a particular class or type of riboswitch and part of a different riboswitch of the same or of any different class or type of riboswitch; part of a riboswitch of any or of a particular class or type of riboswitch and any non-riboswitch sequence or component. Recombinant riboswitches are riboswitches that have been isolated or engineered in a new genetic or nucleic acid context.

Riboswitches can have single or multiple aptamer domains. Aptamer domains in riboswitches having multiple aptamer domains can exhibit cooperative binding of trigger molecules or can not exhibit cooperative binding of trigger molecules (that is, the aptamers need not exhibit cooperative binding). In the latter case, the aptamer domains can be said to be independent binders. Riboswitches having multiple aptamers can have one or multiple expression platform domains. For example, a riboswitch having two aptamer domains that exhibit cooperative binding of their trigger molecules can be linked to a single expression platform domain that is regulated by both aptamer domains. Riboswitches having multiple aptamers can have one or more of the aptamers joined via a linker. Where such aptamers exhibit cooperative binding of trigger molecules, the linker can be a cooperative linker.

Aptamer domains can be said to exhibit cooperative binding if they have a Hill coefficient n between x and x−1, where x is the number of aptamer domains (or the number of binding sites on the aptamer domains) that are being analyzed for cooperative binding. Thus, for example, a riboswitch having two aptamer domains (such as glycine-responsive riboswitches) can be said to exhibit cooperative binding if the riboswitch has Hill coefficient between 2 and 1. It should be understood that the value of x used depends on the number of aptamer domains being analyzed for cooperative binding, not necessarily the number of aptamer domains present in the riboswitch. This makes sense because a riboswitch can have multiple aptamer domains where only some exhibit cooperative binding.

Disclosed are chimeric riboswitches containing heterologous aptamer domains and expression platform domains. That is, chimeric riboswitches are made up an aptamer domain from one source and an expression platform domain from another source. The heterologous sources can be from, for example, different specific riboswitches, different types of riboswitches, or different classes of riboswitches. The heterologous aptamers can also come from non-riboswitch aptamers. The heterologous expression platform domains can also come from non-riboswitch sources.

Modified or derivative riboswitches can be produced using in vitro selection and evolution techniques. In general, in vitro evolution techniques as applied to riboswitches involve producing a set of variant riboswitches where part(s) of the riboswitch sequence is varied while other parts of the riboswitch are held constant. Activation, deactivation or blocking (or other functional or structural criteria) of the set of variant riboswitches can then be assessed and those variant riboswitches meeting the criteria of interest are selected for use or further rounds of evolution. Useful base riboswitches for generation of variants are the specific and consensus riboswitches disclosed herein. Consensus riboswitches can be used to inform which part(s) of a riboswitch to vary for in vitro selection and evolution. The consensus sequence of plant TPP-responsive riboswitches is shown in FIG. 1B.

Also disclosed are modified riboswitches with altered regulation. The regulation of a riboswitch can be altered by operably linking an aptamer domain to the expression platform domain of the riboswitch (which is a chimeric riboswitch). The aptamer domain can then mediate regulation of the riboswitch through the action of, for example, a trigger molecule for the aptamer domain. Aptamer domains can be operably linked to expression platform domains of riboswitches in any suitable manner, including, for example, by replacing the normal or natural aptamer domain of the riboswitch with the new aptamer domain. Generally, any compound or condition that can activate, deactivate or block the riboswitch from which the aptamer domain is derived can be used to activate, deactivate or block the chimeric riboswitch.

Also disclosed are inactivated riboswitches. Riboswitches can be inactivated by covalently altering the riboswitch (by, for example, crosslinking parts of the riboswitch or coupling a compound to the riboswitch). Inactivation of a riboswitch in this manner can result from, for example, an alteration that prevents the trigger molecule for the riboswitch from binding, that prevents the change in state of the riboswitch upon binding of the trigger molecule, or that prevents the expression platform domain of the riboswitch from affecting expression upon binding of the trigger molecule.

Also disclosed are biosensor riboswitches. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, biosensor riboswitches operably linked to a reporter RNA that encodes a protein that serves as or is involved in producing a signal can be used in vivo by engineering a cell or organism to harbor a nucleic acid construct encoding the riboswitch/reporter RNA. An example of a biosensor riboswitch for use in vitro is a riboswitch that includes a conformation dependent label, the signal from which changes depending on the activation state of the riboswitch. Such a biosensor riboswitch preferably uses an aptamer domain from or derived from a naturally occurring riboswitch. Biosensor riboswitches can be used in various situations and platforms. For example, biosensor riboswitches can be used with solid supports, such as plates, chips, strips and wells.

Also disclosed are modified or derivative riboswitches that recognize new trigger molecules. New riboswitches and/or new aptamers that recognize new trigger molecules can be selected for, designed or derived from known riboswitches. This can be accomplished by, for example, producing a set of aptamer variants in a riboswitch, assessing the activation of the variant riboswitches in the presence of a compound of interest, selecting variant riboswitches that were activated (or, for example, the riboswitches that were the most highly or the most selectively activated), and repeating these steps until a variant riboswitch of a desired activity, specificity, combination of activity and specificity, or other combination of properties results.

In general, any aptamer domain can be adapted for use with any expression platform domain by designing or adapting a regulated strand in the expression platform domain to be complementary to the control strand of the aptamer domain. Alternatively, the sequence of the aptamer and control strands of an aptamer domain can be adapted so that the control strand is complementary to a functionally significant sequence in an expression platform.

Disclosed are RNA molecules comprising heterologous riboswitch and coding regions. That is, such RNA molecules are made up of a riboswitch from one source and a coding region from another source. The heterologous sources can be from, for example, different RNA molecules, different transcripts, RNA or transcripts from different genes, RNA or transcripts from different cells, RNA or transcripts from different organisms, RNA or transcripts from different species, natural sequences and artificial or engineered sequences, specific riboswitches, different types of riboswitches, or different classes of riboswitches.

As disclosed herein, the term “coding region” refers to any region of a nucleic acid that codes for amino acids. This can include both a nucleic acid strand that contains the codons or the template for codons and the complement of such a nucleic acid strand in the case of double stranded nucleic acid molecules. Regions of nucleic acids that are not coding regions can be referred to as noncoding regions. Messenger RNA molecules as transcribed typically include noncoding regions at both the 5′ and 3′ ends. Eukaryotic mRNA molecules can also include internal noncoding regions such as introns. Some types of RNA molecules do not include functional coding regions, such as tRNA and rRNA molecules.

1. Aptamer Domains

Aptamers are nucleic acid segments and structures that can bind selectively to particular compounds and classes of compounds. Riboswitches have aptamer domains that, upon binding of a trigger molecule result in a change in the state or structure of the riboswitch. In functional riboswitches, the state or structure of the expression platform domain linked to the aptamer domain changes when the trigger molecule binds to the aptamer domain. Aptamer domains of riboswitches can be derived from any source, including, for example, natural aptamer domains of riboswitches, artificial aptamers, engineered, selected, evolved or derived aptamers or aptamer domains. Aptamers in riboswitches generally have at least one portion that can interact, such as by forming a stem structure, with a portion of the linked expression platform domain. This stem structure will either form or be disrupted upon binding of the trigger molecule.

Consensus aptamer domains of a variety of natural riboswitches are shown in FIG. 11 of U.S. Application Publication No. 2005-0053951 and elsewhere herein. These aptamer domains (including all of the direct variants embodied therein) can be used in riboswitches. The consensus sequences and structures indicate variations in sequence and structure. Aptamer domains that are within the indicated variations are referred to herein as direct variants. These aptamer domains can be modified to produce modified or variant aptamer domains. Conservative modifications include any change in base paired nucleotides such that the nucleotides in the pair remain complementary. Moderate modifications include changes in the length of stems or of loops (for which a length or length range is indicated) of less than or equal to 20% of the length range indicated. Loop and stem lengths are considered to be “indicated” where the consensus structure shows a stem or loop of a particular length or where a range of lengths is listed or depicted. Moderate modifications include changes in the length of stems or of loops (for which a length or length range is not indicated) of less than or equal to 40% of the length range indicated. Moderate modifications also include and functional variants of unspecified portions of the aptamer domain.

Aptamer domains of the disclosed riboswitches can also be used for any other purpose, and in any other context, as aptamers. For example, aptamers can be used to control ribozymes, other molecular switches, and any RNA molecule where a change in structure can affect function of the RNA.

2. Expression Platform Domains

Expression platform domains are a part of riboswitches that affect expression of the RNA molecule that contains the riboswitch. Expression platform domains generally have at least one portion that can interact, such as by forming a stem structure, with a portion of the linked aptamer domain. This stem structure will either form or be disrupted upon binding of the trigger molecule. The stem structure generally either is, or prevents formation of, an expression regulatory structure. An expression regulatory structure is a structure that allows, prevents, enhances or inhibits expression of an RNA molecule containing the structure. Examples include Shine-Dalgarno sequences, initiation codons, transcription terminators, and stability signals, and processing signals, such as RNA splicing junctions and control elements or polyadenylation signals and 3′ terminus signals. For regulation of splicing, it is useful to include a splice junction, an alternative splice junction, and/or a branch site of an intron in the expression platform domain. Interaction of such platform expression domains with sequences in the aptamer domain of a riboswitch can be mediated by complementary sequences between the expression platform domain and the aptamer domain.

### B. Regulated Constructs

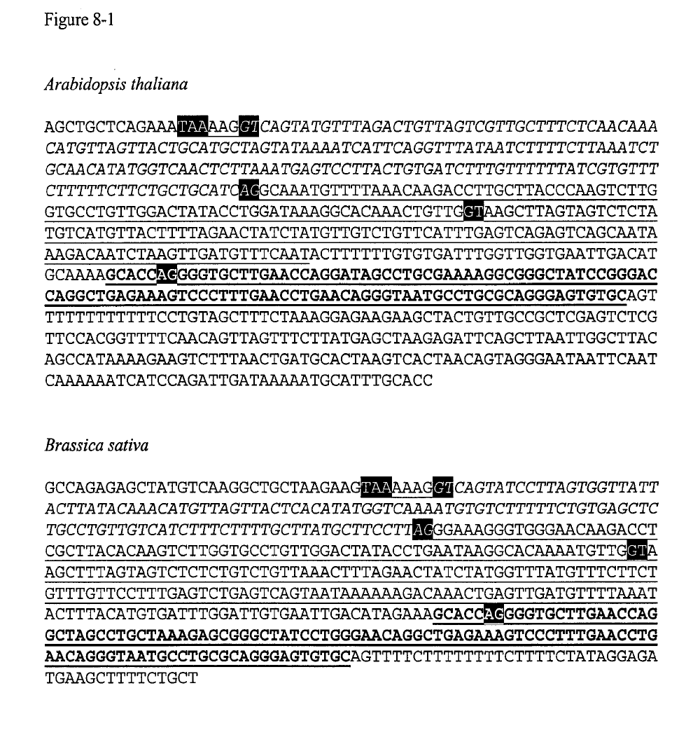

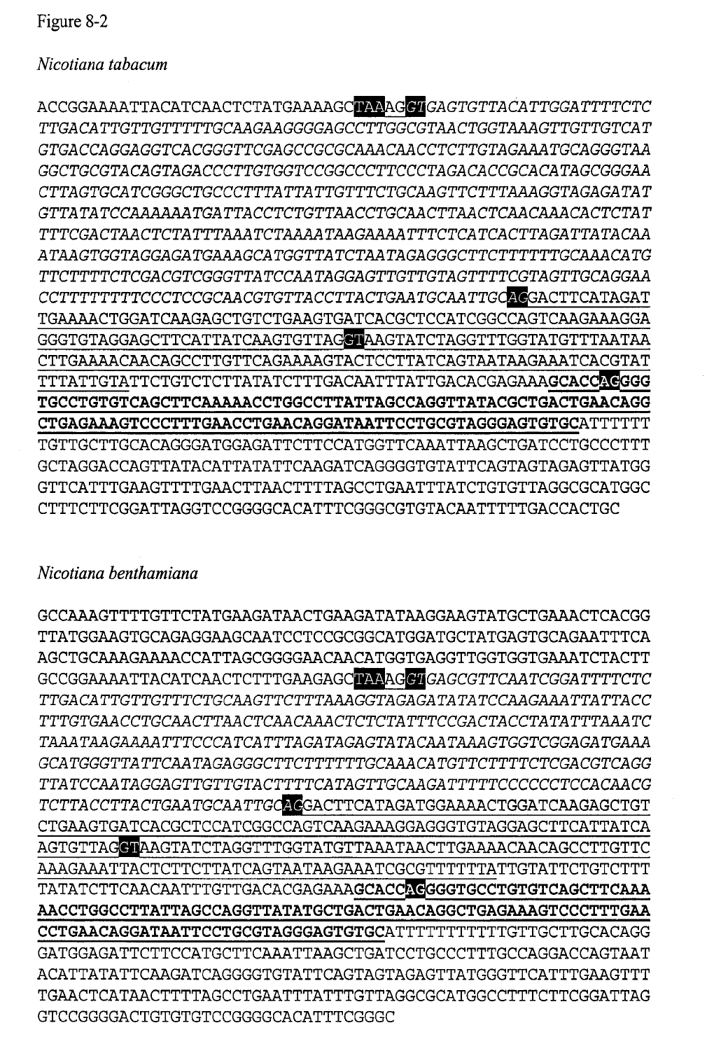

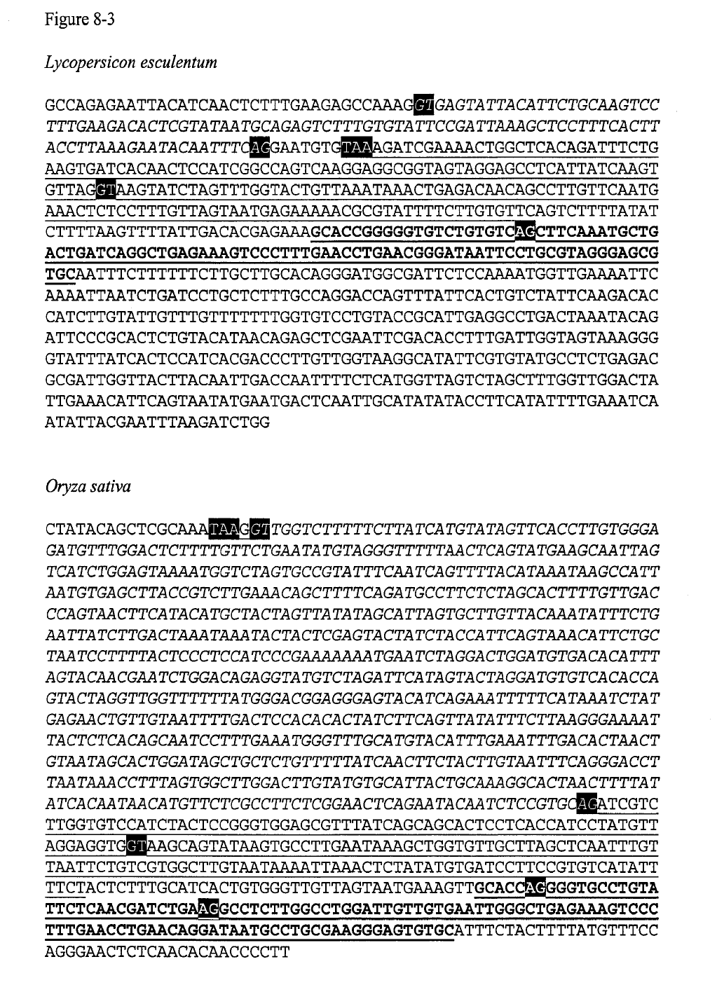

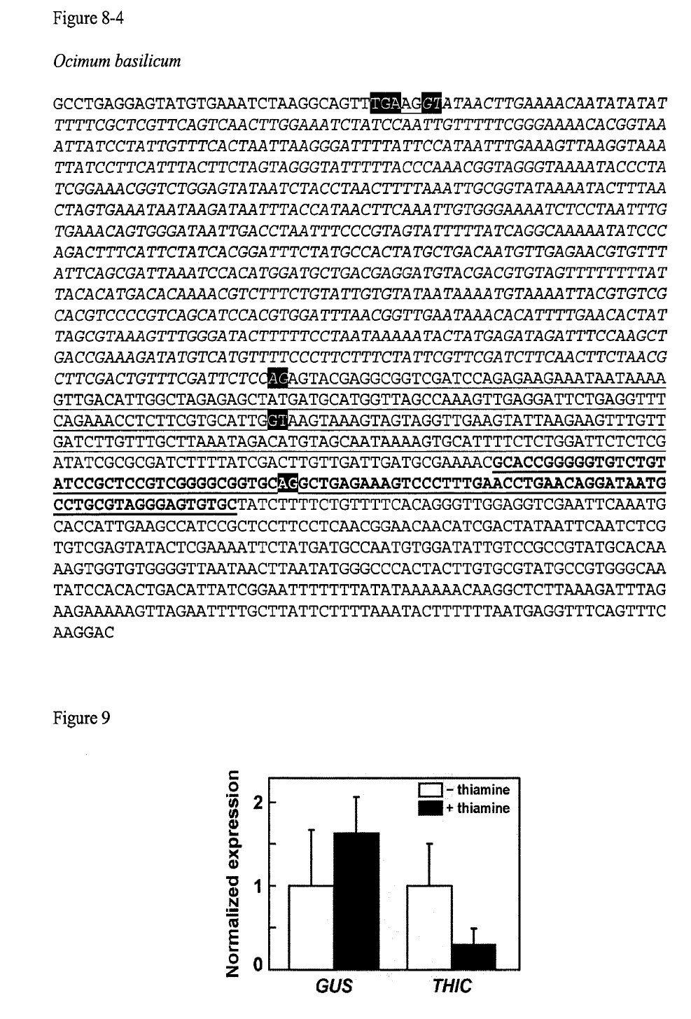

As described elsewhere herein, riboswitches can be used to regulate and affect expression of RNA molecules. The expression platform domain can be operably linked to allow, mediate or facilitate such regulation and control. It can be useful to combine particular sequences and structures in, around or with the expression platform domain sequences. For example, the disclosed TPP riboswitches can be in the 3′ UTR of RNA and in association with an intron in the 3′ UTR. These combined sequences can be referred to as a riboswitch regulated construct or a regulated construct. In this context, the regulated construct can include the riboswitch (comprised of an aptamer domain and an expression platform domain), the regulated intron (which can include expression platform domain and part of the aptamer domain), and other, exonic 3′ UTR sequences. The exonic 3′ UTR sequences may or may not include sequences from the riboswitch. This can depend on, for example, the design of the riboswitch and regulated construct, on whether splicing of the intron takes place or not, or on how RNA processing is affected. For convenience, one of the options—the 3′ UTR sequences in the active and/or predominant form of the RNA—can be referred to as the active 3′ UTR sequence. As an example, the 3′ UTR sequence in form II of the THIC RNA is the active 3′ UTR sequence of these RNAs. Because the disclosed riboswitches and constructs can regulate and affect RNA processing, the regulated construct can also include other sequence that is not part of the riboswitch, the intron or the active 3′ UTR sequence. For example, the disclosed THIC RNAs include sequences between the 3′ terminus sequence of the active 3′ UTR sequence and the aptamer domain of the riboswitch (see FIG. 8). Such sequences can be referred to as spacer 3′ UTR sequences.

The disclosed constructs and RNAs can include a riboswitch, an intron, an active 3′ UTR sequence, and a spacer 3′ UTR sequence. As described above and elsewhere herein, some of these elements and sequences can overlap. Examples of such constructs are described in Example 1 and shown in FIG. 8. FIG. 8 shows examples of naturally-occurring forms of such regulated constructs. It is useful to use the riboswitch, intron, active 3′ UTR sequence, and spacer 3′ UTR sequence from the same naturally-occurring regulated construct. Thus, for example, the entire region from the stop codon to the 3′ end of the riboswitch in a naturally-occurring gene can be used together in a regulated construct operably linked to a heterologous coding sequence. Examples of such constructs are described in Example 1. Alternatively, different sequences from different regulated constructs can be substituted or a different or derivative riboswitch or aptamer domain can be combined with other introns, active 3′ UTR sequences, and/or spacer 3′ UTR sequences. For example, a consensus or derivative aptamer domain can be used in a regulated construct.

### C. Trigger Molecules

Trigger molecules are molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques).

### D. Compounds

Also disclosed are compounds, and compositions containing such compounds, that can activate, deactivate or block a riboswitch. Riboswitches function to control gene expression through the binding or removal of a trigger molecule. Compounds can be used to activate, deactivate or block a riboswitch. The trigger molecule for a riboswitch (as well as other activating compounds) can be used to activate a riboswitch. Compounds other than the trigger molecule generally can be used to deactivate or block a riboswitch. Riboswitches can also be deactivated by, for example, removing trigger molecules from the presence of the riboswitch. A riboswitch can be blocked by, for example, binding of an analog of the trigger molecule that does not activate the riboswitch.

Also disclosed are compounds for altering expression of an RNA molecule (such as by altering spicing or processing of the RNA), or of a gene encoding an RNA molecule, where the RNA molecule includes a riboswitch. This can be accomplished by bringing a compound into contact with the RNA molecule. Riboswitches function to control gene expression through the binding or removal of a trigger molecule. Thus, subjecting an RNA molecule of interest that includes a riboswitch to conditions that activate, deactivate or block the riboswitch can be used to alter expression of the RNA (such as by altering spicing or processing of the RNA). Expression can be altered as a result of, for example, termination of transcription or blocking of ribosome binding to the RNA. Binding of a trigger molecule can, depending on the nature of the riboswitch, reduce or prevent expression of the RNA molecule or promote or increase expression of the RNA molecule.

Also disclosed are compounds for regulating expression of an RNA molecule, or of a gene encoding an RNA molecule. Also disclosed are compounds for regulating expression of a naturally occurring gene or RNA that contains a riboswitch by activating, deactivating or blocking the riboswitch. If the gene is essential for survival of a cell or organism that harbors it, activating, deactivating or blocking the riboswitch can in death, stasis or debilitation of the cell or organism.

Also disclosed are compounds for regulating expression of an isolated, engineered or recombinant gene or RNA that contains a riboswitch by activating, deactivating or blocking the riboswitch. Since the riboswitches disclosed herein control alternative splicing, activating, deactivating, or blocking the riboswitch can regulate expression of a gene. An advantage of riboswitches as the primary control for such regulation is that riboswitch trigger molecules can be small, non-antigenic molecules.

Also disclosed are methods of identifying compounds that activate, deactivate or block a riboswitch. For examples, compounds that activate a riboswitch can be identified by bringing into contact a test compound and a riboswitch and assessing activation of the riboswitch. If the riboswitch is activated, the test compound is identified as a compound that activates the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

Identification of compounds that block a riboswitch can be accomplished in any suitable manner. For example, an assay can be performed for assessing activation or deactivation of a riboswitch in the presence of a compound known to activate or deactivate the riboswitch and in the presence of a test compound. If activation or deactivation is not observed as would be observed in the absence of the test compound, then the test compound is identified as a compound that blocks activation or deactivation of the riboswitch.

Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

Specific compounds that can be used to activate riboswitches are also disclosed. Compounds useful with TPP-responsive riboswitches include compounds having the formula:

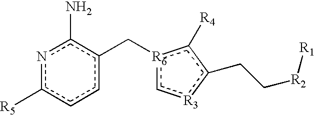

where the compound can bind a TPP-responsive riboswitch or derivative thereof, where R₁ is positively charged, where R₂ and R₃ are each independently C, O, or S, where R₄ is CH₃, NH₂, OH, SH, H or not present, where R₅ is CH₃, NH₂, OH, SH, or H, where R₆ is C or N, and where [symbol] each independently represent a single or double bond. Also contemplated are compounds as defined above where R₁ is phosphate, diphosphate or triphosphate.

Every compound within the above definition is intended to be and should be considered to be specifically disclosed herein. Further, every subgroup that can be identified within the above definition is intended to be and should be considered to be specifically disclosed herein. As a result, it is specifically contemplated that any compound or subgroup of compounds can be either specifically included for or excluded from use or included in or excluded from a list of compounds. For example, as one option, a group of compounds is contemplated where each compound is as defined above but is not TPP, TP or thiamine. As another example, a group of compounds is contemplated where each compound is as defined above and is able to activate a TPP-responsive riboswitch. Thiamine pyrophosphate (TPP) is the trigger molecule for TPP-responsive riboswitches and can active TPP-responsive riboswitches. Pyrithiamine pyrophosphate can active TPP-responsive riboswitches. Pyrithiamine and pyrithiamine pyrophosphate can be independently and specifically included or excluded from the compounds, trigger molecules and methods disclosed herein. Thiamine and thiamine pyrophosphate can be independently and specifically included or excluded from the compounds, trigger molecules and methods disclosed herein.

### E. Constructs, Vectors and Expression Systems

The disclosed riboswitches can be used with any suitable expression system. Recombinant expression is usefully accomplished using a vector, such as a plasmid. The vector can include a promoter operably linked to riboswitch-encoding sequence and RNA to be expression (e.g., RNA encoding a protein). The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes. A variety of prokaryotic and eukaryotic expression vectors suitable for carrying riboswitch-regulated constructs can be produced. Such expression vectors include, for example, pET, pET3d, pCR2.1, pBAD, pUC, and yeast vectors. The vectors can be used, for example, in a variety of in vivo and in vitro situation.

Viral vectors include adenovirus, adeno-associated virus, herpes virus, vaccinia virus, polio virus, AIDS virus, neuronal trophic virus, Sindbis and other RNA viruses, including these viruses with the HIV backbone. Also useful are any viral families which share the properties of these viruses which make them suitable for use as vectors. Retroviral vectors, which are described in Verma (1985), include Murine Maloney Leukemia virus, MMLV, and retroviruses that express the desirable properties of MMLV as a vector. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA.

A “promoter” is generally a sequence or sequences of DNA that function when in a relatively fixed location in regard to the transcription start site. A “promoter” contains core elements required for basic interaction of RNA polymerase and transcription factors and can contain upstream elements and response elements.

“Enhancer” generally refers to a sequence of DNA that functions at no fixed distance from the transcription start site and can be either 5′ (Laimins, 1981) or 3′ (Lusky et al., 1983) to the transcription unit. Furthermore, enhancers can be within an intron (Banerji et al., 1983) as well as within the coding sequence itself (Osborne et al., 1984). They are usually between 10 and 300 by in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The vector can include nucleic acid sequence encoding a marker product. This marker product is used to determine if the gene has been delivered to the cell and once delivered is being expressed. Preferred marker genes are the E. coli lacZ gene which encodes β-galactosidase and green fluorescent protein.

In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern and Berg, 1982), mycophenolic acid, (Mulligan and Berg, 1980) or hygromycin (Sugden et al., 1985).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991).

1. Viral Vectors

Preferred viral vectors are Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Preferred retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not useful in non-proliferating cells. Adenovirus vectors are relatively stable and easy to work with, have high titers, and can be delivered in aerosol formulation, and can transfect non-dividing cells. Pox viral vectors are large and have several sites for inserting genes; they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors have higher transaction (ability to introduce genes) abilities than do most chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

i. Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

ii. Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A preferred viral vector is one based on an adenovirus which has had the E1 gene removed and these virons are generated in a cell line such as the human 293 cell line. In another preferred embodiment both the E1 and E3 genes are removed from the adenovirus genome.

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and can contain upstream elements and response elements.

2. Viral Promoters and Enhancers

Preferred promoters controlling transcription from vectors in mammalian host cells can be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 by in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus. Preferred examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer can be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

It is preferred that the promoter and/or enhancer region be active in all eukaryotic cell types. A preferred promoter of this type is the CMV promoter (650 bases). Other preferred promoters are SV40 promoters, cytomegalovirus (full length promoter), and retroviral vector LTF.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) can also contain sequences necessary for the termination of transcription which can affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In a preferred embodiment of the transcription unit, the polyadenylation region is derived from the SV40 early polyadenylation signal and consists of about 400 bases. It is also preferred that the transcribed units contain other standard sequences alone or in combination with the above sequences improve expression from, or stability of, the construct.

3. Markers

The vectors can include nucleic acid sequence encoding a marker product. This marker product is used to determine if the gene has been delivered to the cell and once delivered is being expressed. Preferred marker genes are the E. coli lacZ gene which encodes β-galactosidase and green fluorescent protein.

In some embodiments the marker can be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR⁻ cells and mouse LTK⁻ cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

### F. Biosensor Riboswitches

Also disclosed are biosensor riboswitches. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, riboswitches that control alternative splicing can be operably linked to a reporter RNA that encodes a protein that serves as or is involved in producing a signal can be used in vivo by engineering a cell or organism to harbor a nucleic acid construct encoding the riboswitch. An example of a biosensor riboswitch for use in vitro is a riboswitch that includes a conformation dependent label, the signal from which changes depending on the activation state of the riboswitch. Such a biosensor riboswitch preferably uses an aptamer domain from or derived from a naturally occurring riboswitch.

### G. Reporter Proteins and Peptides

For assessing activation of a riboswitch, or for biosensor riboswitches, a reporter protein or peptide can be used. The reporter protein or peptide can be encoded by the RNA the expression of which is regulated by the riboswitch. The examples describe the use of some specific reporter proteins. The use of reporter proteins and peptides is well known and can be adapted easily for use with riboswitches. The reporter proteins can be any protein or peptide that can be detected or that produces a detectable signal. Preferably, the presence of the protein or peptide can be detected using standard techniques (e.g., radioimmunoassay, radio-labeling, immunoassay, assay for enzymatic activity, absorbance, fluorescence, luminescence, and Western blot). More preferably, the level of the reporter protein is easily quantifiable using standard techniques even at low levels. Useful reporter proteins include luciferases, green fluorescent proteins and their derivatives, such as firefly luciferase (FL) from Photinus pyralis, and Renilla luciferase (RL) from Renilla reniformis.

### H. Conformation Dependent Labels

Conformation dependent labels refer to all labels that produce a change in fluorescence intensity or wavelength based on a change in the form or conformation of the molecule or compound (such as a riboswitch) with which the label is associated. Examples of conformation dependent labels used in the context of probes and primers include molecular beacons, Amplifluors, FRET probes, cleavable FRET probes, TaqMan probes, scorpion primers, fluorescent triplex oligos including but not limited to triplex molecular beacons or triplex FRET probes, fluorescent water-soluble conjugated polymers, PNA probes and QPNA probes. Such labels, and, in particular, the principles of their function, can be adapted for use with riboswitches. Several types of conformation dependent labels are reviewed in Schweitzer and Kingsmore, Curr. Opin. Biotech. 12:21-27 (2001).

Stem quenched labels, a form of conformation dependent labels, are fluorescent labels positioned on a nucleic acid such that when a stem structure forms a quenching moiety is brought into proximity such that fluorescence from the label is quenched. When the stem is disrupted (such as when a riboswitch containing the label is activated), the quenching moiety is no longer in proximity to the fluorescent label and fluorescence increases. Examples of this effect can be found in molecular beacons, fluorescent triplex oligos, triplex molecular beacons, triplex FRET probes, and QPNA probes, the operational principles of which can be adapted for use with riboswitches.

Stem activated labels, a form of conformation dependent labels, are labels or pairs of labels where fluorescence is increased or altered by formation of a stem structure. Stem activated labels can include an acceptor fluorescent label and a donor moiety such that, when the acceptor and donor are in proximity (when the nucleic acid strands containing the labels form a stem structure), fluorescence resonance energy transfer from the donor to the acceptor causes the acceptor to fluoresce. Stem activated labels are typically pairs of labels positioned on nucleic acid molecules (such as riboswitches) such that the acceptor and donor are brought into proximity when a stem structure is formed in the nucleic acid molecule. If the donor moiety of a stem activated label is itself a fluorescent label, it can release energy as fluorescence (typically at a different wavelength than the fluorescence of the acceptor) when not in proximity to an acceptor (that is, when a stem structure is not formed). When the stem structure forms, the overall effect would then be a reduction of donor fluorescence and an increase in acceptor fluorescence. FRET probes are an example of the use of stem activated labels, the operational principles of which can be adapted for use with riboswitches.

### I. Detection Labels

To aid in detection and quantitation of riboswitch activation, deactivation or blocking, or expression of nucleic acids or protein produced upon activation, deactivation or blocking of riboswitches, detection labels can be incorporated into detection probes or detection molecules or directly incorporated into expressed nucleic acids or proteins. As used herein, a detection label is any molecule that can be associated with nucleic acid or protein, directly or indirectly, and which results in a measurable, detectable signal, either directly or indirectly. Many such labels are known to those of skill in the art. Examples of detection labels suitable for use in the disclosed method are radioactive isotopes, fluorescent molecules, phosphorescent molecules, enzymes, antibodies, and ligands.

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as Quantum Dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Polyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Useful fluorescent labels are fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

Labeled nucleotides are a useful form of detection label for direct incorporation into expressed nucleic acids during synthesis. Examples of detection labels that can be incorporated into nucleic acids include nucleotide analogs such as BrdUrd (5-bromodeoxyuridine, Hoy and Schimke, Mutation Research 290:217-230 (1993)), aminoallyldeoxyuridine (Henegariu et al., Nature Biotechnology 18:345-348 (2000)), 5-methylcytosine (Sano et al., Biochim. Biophys. Acta 951:157-165 (1988)), bromouridine (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (bromodeoxyuridine, BrdUrd, BrdU, BUdR, Sigma-Aldrich Co). Other useful nucleotide analogs for incorporation of detection label into DNA are AA-dUTP (aminoallyl-deoxyuridine triphosphate, Sigma-Aldrich Co.), and 5-methyl-dCTP (Roche Molecular Biochemicals). A useful nucleotide analog for incorporation of detection label into RNA is biotin-16-UTP (biotin-16-uridine-5′-triphosphate, Roche Molecular Biochemicals). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2-dioxetane-3-2′-(5′-chloro)tricyclo[3.3.1.1^{3,7}]decane]-4-yl)phenyl phosphate; Tropix, Inc.). Labels can also be enzymes, such as alkaline phosphatase, soybean peroxidase, horseradish peroxidase and polymerases, that can be detected, for example, with chemical signal amplification or by using a substrate to the enzyme which produces light (for example, a chemiluminescent 1,2-dioxetane substrate) or fluorescent signal.

Molecules that combine two or more of these detection labels are also considered detection labels. Any of the known detection labels can be used with the disclosed probes, tags, molecules and methods to label and detect activated or deactivated riboswitches or nucleic acid or protein produced in the disclosed methods. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. As used herein, detection molecules are molecules which interact with a compound or composition to be detected and to which one or more detection labels are coupled.

### J. Sequence Similarities

It is understood that as discussed herein the use of the terms homology and identity mean the same thing as similarity. Thus, for example, if the use of the word homology is used between two sequences (non-natural sequences, for example) it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed riboswitches, aptamers, expression platforms, genes and proteins herein, is through defining the variants and derivatives in terms of homology to specific known sequences. This identity of particular sequences disclosed herein is also discussed elsewhere herein. In general, variants of riboswitches, aptamers, expression platforms, genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to a stated sequence or a native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison can be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods can differ, but the skilled artisan understands if identity is found with at least one of these methods, the sequences would be said to have the stated identity.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

### K. Hybridization and Selective Hybridization

The term hybridization typically means a sequence driven interaction between at least two nucleic acid molecules, such as a primer or a probe and a riboswitch or a gene. Sequence driven interaction means an interaction that occurs between two nucleotides or nucleotide analogs or nucleotide derivatives in a nucleotide specific manner. For example, G interacting with C and A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization can involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting nucleic acid is in for example, 10 or 100 or 1000 fold excess. This type of assay can be performed at under conditions where both the limiting and non-limiting nucleic acids are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of nucleic acid that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the nucleic acid molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions can provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### L. Nucleic Acids

There are a variety of molecules disclosed herein that are nucleic acid based, including, for example, riboswitches, aptamers, and nucleic acids that encode riboswitches and aptamers. The disclosed nucleic acids can be made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if a nucleic acid molecule is introduced into a cell or cell environment through for example exogenous delivery, it is advantageous that the nucleic acid molecule be made up of nucleotide analogs that reduce the degradation of the nucleic acid molecule in the cellular environment.

So long as their relevant function is maintained, riboswitches, aptamers, expression platforms and any other oligonucleotides and nucleic acids can be made up of or include modified nucleotides (nucleotide analogs). Many modified nucleotides are known and can be used in oligonucleotides and nucleic acids. A nucleotide analog is a nucleotide which contains some type of modification to the base, sugar, or phosphate moieties. Modifications to the base moiety would include natural and synthetic modifications of A, C, G, and T/U as well as different purine or pyrimidine bases, such as uracil-5-yl, hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993. Certain nucleotide analogs, such as 5-substituted pyrimidines, 6-azapyrimidines and N-2, N-6 and O-6 substituted purines, including 2-aminopropyladenine, 5-propynyluracil, 5-propynylcytosine, and 5-methylcytosine can increase the stability of duplex formation. Other modified bases are those that function as universal bases. Universal bases include 3-nitropyrrole and 5-nitroindole. Universal bases substitute for the normal bases but have no bias in base pairing. That is, universal bases can base pair with any other base. Base modifications often can be combined with for example a sugar modification, such as 2′-O-methoxyethyl, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121; 5,596,091; 5,614,617; and 5,681,941, which detail and describe a range of base modifications. Each of these patents is herein incorporated by reference in its entirety, and specifically for their description of base modifications, their synthesis, their use, and their incorporation into oligonucleotides and nucleic acids.

Nucleotide analogs can also include modifications of the sugar moiety. Modifications to the sugar moiety would include natural modifications of the ribose and deoxyribose as well as synthetic modifications. Sugar modifications include but are not limited to the following modifications at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C1 to C10, alkyl or C2 to C10 alkenyl and alkynyl. 2′ sugar modifications also include but are not limited to —O[(CH₂)nO]m CH₃, —O(CH₂)n OCH₃, —O(CH₂)n NH₂, —O(CH₂)n CH₃, —O(CH₂)n-ONH₂, and —O(CH₂)nON[(CH₂)n CH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂, CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications can also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH₂ and S, Nucleotide sugar analogs can also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety, and specifically for their description of modified sugar structures, their synthesis, their use, and their incorporation into nucleotides, oligonucleotides and nucleic acids.

Nucleotide analogs can also be modified at the phosphate moiety. Modified phosphate moieties include but are not limited to those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference its entirety, and specifically for their description of modified phosphates, their synthesis, their use, and their incorporation into nucleotides, oligonucleotides and nucleic acids.

It is understood that nucleotide analogs need only contain a single modification, but can also contain multiple modifications within one of the moieties or between different moieties.

Nucleotide substitutes are molecules having similar functional properties to nucleotides, but which do not contain a phosphate moiety, such as peptide nucleic acid (PNA). Nucleotide substitutes are molecules that will recognize and hybridize to (base pair to) complementary nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference its entirety, and specifically for their description of phosphate replacements, their synthesis, their use, and their incorporation into nucleotides, oligonucleotides and nucleic acids.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference (See also Nielsen et al., Science 254:1497-1500 (1991)).

Oligonucleotides and nucleic acids can be comprised of nucleotides and can be made up of different types of nucleotides or the same type of nucleotides. For example, one or more of the nucleotides in an oligonucleotide can be ribonucleotides, 2′-O-methyl ribonucleotides, or a mixture of ribonucleotides and 2′-O-methyl ribonucleotides; about 10% to about 50% of the nucleotides can be ribonucleotides, 2′-O-methyl ribonucleotides, or a mixture of ribonucleotides and 2′-O-methyl ribonucleotides; about 50% or more of the nucleotides can be ribonucleotides, 2′-O-methyl ribonucleotides, or a mixture of ribonucleotides and 2′-O-methyl ribonucleotides; or all of the nucleotides are ribonucleotides, 2′-O-methyl ribonucleotides, or a mixture of ribonucleotides and 2′-O-methyl ribonucleotides. Such oligonucleotides and nucleic acids can be referred to as chimeric oligonucleotides and chimeric nucleic acids.

### M. Solid Supports

Solid supports are solid-state substrates or supports with which molecules (such as trigger molecules) and riboswitches (or other components used in, or produced by, the disclosed methods) can be associated. Riboswitches and other molecules can be associated with solid supports directly or indirectly. For example, analytes (e.g., trigger molecules, test compounds) can be bound to the surface of a solid support or associated with capture agents (e.g., compounds or molecules that bind an analyte) immobilized on solid supports. As another example, riboswitches can be bound to the surface of a solid support or associated with probes immobilized on solid supports. An array is a solid support to which multiple riboswitches, probes or other molecules have been associated in an array, grid, or other organized pattern.

Solid-state substrates for use in solid supports can include any solid material with which components can be associated, directly or indirectly. This includes materials such as acrylamide, agarose, cellulose, nitrocellulose, glass, gold, polystyrene, polyethylene vinyl acetate, polypropylene, polymethacrylate, polyethylene, polyethylene oxide, polysilicates, polycarbonates, teflon, fluorocarbons, nylon, silicon rubber, polyanhydrides, polyglycolic acid, polylactic acid, polyorthoesters, functionalized silane, polypropylfumerate, collagen, glycosaminoglycans, and polyamino acids. Solid-state substrates can have any useful form including thin film, membrane, bottles, dishes, fibers, woven fibers, shaped polymers, particles, beads, microparticles, or a combination. Solid-state substrates and solid supports can be porous or non-porous. A chip is a rectangular or square small piece of material. Preferred forms for solid-state substrates are thin films, beads, or chips. A useful form for a solid-state substrate is a microtiter dish. In some embodiments, a multiwell glass slide can be employed.

An array can include a plurality of riboswitches, trigger molecules, other molecules, compounds or probes immobilized at identified or predefined locations on the solid support. Each predefined location on the solid support generally has one type of component (that is, all the components at that location are the same). Alternatively, multiple types of components can be immobilized in the same predefined location on a solid support. Each location will have multiple copies of the given components. The spatial separation of different components on the solid support allows separate detection and identification.

Although useful, it is not required that the solid support be a single unit or structure. A set of riboswitches, trigger molecules, other molecules, compounds and/or probes can be distributed over any number of solid supports. For example, at one extreme, each component can be immobilized in a separate reaction tube or container, or on separate beads or microparticles.

Methods for immobilization of oligonucleotides to solid-state substrates are well established. Oligonucleotides, including address probes and detection probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A useful method of attaching oligonucleotides to solid-state substrates is described by Guo et al., Nucleic Acids Res. 22:5456-5465 (1994).

Each of the components (for example, riboswitches, trigger molecules, or other molecules) immobilized on the solid support can be located in a different predefined region of the solid support. The different locations can be different reaction chambers. Each of the different predefined regions can be physically separated from each other of the different regions. The distance between the different predefined regions of the solid support can be either fixed or variable. For example, in an array, each of the components can be arranged at fixed distances from each other, while components associated with beads will not be in a fixed spatial relationship. In particular, the use of multiple solid support units (for example, multiple beads) will result in variable distances.

Components can be associated or immobilized on a solid support at any density. Components can be immobilized to the solid support at a density exceeding 400 different components per cubic centimeter. Arrays of components can have any number of components. For example, an array can have at least 1,000 different components immobilized on the solid support, at least 10,000 different components immobilized on the solid support, at least 100,000 different components immobilized on the solid support, or at least 1,000,000 different components immobilized on the solid support.

### N. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for detecting compounds, the kit comprising one or more biosensor riboswitches. The kits also can contain reagents and labels for detecting activation of the riboswitches.

### O. Mixtures

Disclosed are mixtures formed by performing or preparing to perform the disclosed method. For example, disclosed are mixtures comprising riboswitches and trigger molecules.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### P. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. For example, disclosed and contemplated are systems comprising biosensor riboswitches, a solid support and a signal-reading device.

### Q. Data Structures and Computer Control

Disclosed are data structures used in, generated by, or generated from, the disclosed method. Data structures generally are any form of data, information, and/or objects collected, organized, stored, and/or embodied in a composition or medium. Riboswitch structures and activation measurements stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Methods

Disclosed herein are methods for affecting processing of RNA comprising introducing into the RNA a construct comprising a riboswitch, wherein the riboswitch is capable of regulating splicing of RNA, wherein regulation of splicing affects processing of the RNA. The riboswitch can, for example, regulate alternative splicing. The riboswitch can comprise an aptamer domain and an expression platform domain, wherein the aptamer domain and the expression platform domain are heterologous. The riboswitch can be in an intron of the RNA. The riboswitch can be activated by a trigger molecule, such as TPP. The riboswitch can be a TPP-responsive riboswitch. The riboswitch can activate alternative splicing. The riboswitch can repress alternative splicing. The splicing can occur non-naturally. The region of the aptamer with alternative splicing control can be found, for example, in loop 5. The region of the aptamer with alternative splicing control can also found, for example, in stem P2. The splice sites can be located, for example, at positions between −130 to −160 relative to the 5′ end of the aptamer.

By “regulating splicing of RNA” is meant a riboswitch that can control splicing of RNA, thereby causing a different mRNA molecule to be formed, and potentially (though not always) a different protein. The riboswitch can, for example, regulate alternative splicing. By “affecting RNA processing” is meant a riboswitch that can affect RNA processing, thereby causing a different mRNA molecule to be formed, and potentially (though not always) altering expression of the RNA. The riboswitch can, for example, regulate transcription termination, formation of the 3′ terminus of an RNA or polyadenylation of an RNA.

Further disclosed are methods for activating, deactivating or blocking a riboswitch that regulates splicing of RNA and/or affects RNA processing. Such methods can involve, for example, bringing into contact a riboswitch and a compound or trigger molecule that can activate, deactivate or block the riboswitch. Riboswitches function to control gene expression through the binding or removal of a trigger molecule. Compounds can be used to activate, deactivate or block a riboswitch. The trigger molecule for a riboswitch (as well as other activating compounds) can be used to activate a riboswitch. Compounds other than the trigger molecule generally can be used to deactivate or block a riboswitch (such as TPP). Riboswitches can also be deactivated by, for example, removing trigger molecules from the presence of the riboswitch. Thus, the disclosed method of deactivating a riboswitch can involve, for example, removing a trigger molecule (or other activating compound) from the presence or contact with the riboswitch. A riboswitch can be blocked by, for example, binding of an analog of the trigger molecule that does not activate the riboswitch.

Also disclosed are methods for altering expression of an RNA molecule, or of a gene encoding an RNA molecule, where the RNA molecule includes a riboswitch that regulates splicing, by bringing a compound into contact with the RNA molecule. The riboswitch can, for example, regulate alternative spicing of the RNA molecule and/or affect processing of the RNA molecule. Riboswitches function to control gene expression through the binding or removal of a trigger molecule. Thus, subjecting an RNA molecule of interest that includes a riboswitch to conditions that activate, deactivate or block the riboswitch can be used to alter expression of the RNA. Expression can be altered as a result of, for example, termination of transcription or blocking of ribosome binding to the RNA. Binding of a trigger molecule can, depending on the nature of the riboswitch and the type of splicing or processing that occurs, reduce or prevent expression of the RNA molecule or promote or increase expression of the RNA molecule.

Also disclosed are methods for regulating expression of a naturally occurring gene or RNA that contains a riboswitch that regulates splicing by activating, deactivating or blocking the riboswitch. The riboswitch can regulate, for example, alternative spicing of the RNA. If the gene is essential for survival of a cell or organism that harbors it, activating, deactivating or blocking the riboswitch can result in death, stasis or debilitation of the cell or organism. For example, activating a naturally occurring riboswitch in a naturally occurring gene that is essential to survival of a plant can result in death of the plant (if activation of the riboswitch controls alternative splicing and/or affects RNA processing, which in turn up-regulates or down-regulates a crucial protein).

Also disclosed are methods for selecting and identifying compounds that can activate, deactivate or block a riboswitch that regulates splicing. The riboswitch can regulate, for example, alternative spicing. Activation of a riboswitch refers to the change in state of the riboswitch upon binding of a trigger molecule. A riboswitch can be activated by compounds other than the trigger molecule and in ways other than binding of a trigger molecule. The term trigger molecule is used herein to refer to molecules and compounds that can activate a riboswitch. This includes the natural or normal trigger molecule for the riboswitch and other compounds that can activate the riboswitch. Natural or normal trigger molecules are the trigger molecule for a given riboswitch in nature or, in the case of some non-natural riboswitches, the trigger molecule for which the riboswitch was designed or with which the riboswitch was selected (as in, for example, in vitro selection or in vitro evolution techniques). Non-natural trigger molecules can be referred to as non-natural trigger molecules.

Also disclosed are methods of identifying compounds that activate, deactivate or block a riboswitch that regulates splicing and/or affects RNA processing. For example, compounds that activate a riboswitch can be identified by bringing into contact a test compound and a riboswitch and assessing activation of the riboswitch by measuring the splicing and/or processing of the RNA, or measuring the differential level of the protein expressed as a result of the splicing and/or processing event. If the riboswitch is activated, the test compound is identified as a compound that activates the riboswitch. Activation of a riboswitch can be assessed in any suitable manner. For example, the riboswitch can be linked to a reporter RNA and expression, expression level, or change in expression level of the reporter RNA can be measured in the presence and absence of the test compound. As another example, the riboswitch can include a conformation dependent label, the signal from which changes depending on the activation state of the riboswitch. Such a riboswitch preferably uses an aptamer domain from or derived from a naturally occurring riboswitch. As can be seen, assessment of activation of a riboswitch can be performed with the use of a control assay or measurement or without the use of a control assay or measurement. Methods for identifying compounds that deactivate a riboswitch can be performed in analogous ways.

In addition to the methods disclosed elsewhere herein, identification of compounds that block a riboswitch that regulates splicing and/or affects RNA processing can be accomplished in any suitable manner. For example, an assay can be performed for assessing activation or deactivation of a riboswitch in the presence of a compound known to activate or deactivate the riboswitch and in the presence of a test compound. If activation or deactivation is not observed as would be observed in the absence of the test compound, then the test compound is identified as a compound that blocks activation or deactivation of the riboswitch.

Also disclosed are methods of detecting compounds using biosensor riboswitches that regulate alternative splicing. The method can include bringing into contact a test sample and a biosensor riboswitch and assessing the activation of the biosensor riboswitch. Activation of the biosensor riboswitch indicates the presence of the trigger molecule for the biosensor riboswitch in the test sample. Biosensor riboswitches are engineered riboswitches that produce a detectable signal in the presence of their cognate trigger molecule. Useful biosensor riboswitches can be triggered at or above threshold levels of the trigger molecules. Biosensor riboswitches can be designed for use in vivo or in vitro. For example, biosensor riboswitches that regulate alternative binding can be operably linked to a reporter RNA that encodes a protein that serves as or is involved in producing a signal that can be used in vivo by engineering a cell or organism to harbor a nucleic acid construct encoding the riboswitch/reporter RNA. An example of a biosensor riboswitch for use in vitro is riboswitch that includes a conformation dependent label, the signal from which changes depending on the activation state of the riboswitch. Such a biosensor riboswitch preferably uses an aptamer domain from or derived from a naturally occurring TPP riboswitch.

Also disclosed are compounds made by identifying a compound that activates, deactivates or blocks a riboswitch and manufacturing the identified compound. This can be accomplished by, for example, combining compound identification methods as disclosed elsewhere herein with methods for manufacturing the identified compounds. For example, compounds can be made by bringing into contact a test compound and a riboswitch, assessing activation of the riboswitch, and, if the riboswitch is activated by the test compound, manufacturing the test compound that activates the riboswitch as the compound.

Also disclosed are compounds made by checking activation, deactivation or blocking of a riboswitch by a compound and manufacturing the checked compound. This can be accomplished by, for example, combining compound activation, deactivation or blocking assessment methods as disclosed elsewhere herein with methods for manufacturing the checked compounds. For example, compounds can be made by bringing into contact a test compound and a riboswitch, assessing activation of the riboswitch, and, if the riboswitch is activated by the test compound, manufacturing the test compound that activates the riboswitch as the compound. Checking compounds for their ability to activate, deactivate or block a riboswitch refers to both identification of compounds previously unknown to activate, deactivate or block a riboswitch and to assessing the ability of a compound to activate, deactivate or block a riboswitch where the compound was already known to activate, deactivate or block the riboswitch.

A compound can be identified as activating a riboswitch or can be deter mined to have riboswitch activating activity if the signal in a riboswitch assay is increased in the presence of the compound by at least 1 fold, 2 fold, 3 fold, 4 fold, 5 fold, 50%, 75%, 100%, 125%, 150%, 175%, 200%, 250%, 300%, 400%, or 500% compared to the same riboswitch assay in the absence of the compound (that is, compared to a control assay). The riboswitch assay can be performed using any suitable riboswitch construct. Riboswitch constructs that are particularly useful for riboswitch activation assays are described elsewhere herein. The identification of a compound as activating a riboswitch or as having a riboswitch activation activity can be made in terms of one or more particular riboswitches, riboswitch constructs or classes of riboswitches. For convenience, compounds identified as activating a riboswitch that controls alternative splicing can be so identified for particular riboswitches.

### EXAMPLES

### A. Example 1

### Riboswitch Control of Gene Expression in Plants by Alternative 3′ End Processing of mRNAs

The most widespread riboswitch class found in organisms from all three domains of life is responsive to the coenzyme thiamin pyrophosphate (TPP), which is a derivative of vitamin B₁. It was discovered that TPP riboswitches are present in the 3′ untranslated region (UTR) of the thiamin biosynthetic gene THIC of all plant species examined. The THIC TPP riboswitch controls the formation of transcripts with alternative 3′ UTR lengths, which affect mRNA stability and protein production. It has been demonstrated that riboswitch-mediated regulation of alternative 3′ end processing is critical for TPP-dependent feedback control of THIC expression. The data reveal a mechanism whereby metabolite-dependent alteration of RNA folding controls splicing and alternative 3′ end processing of mRNAs. These findings highlight the importance of metabolite sensing by riboswitches in plants and further reveals the significance of alternative 3′ end processing as a mechanism of gene control in eukaryotes.

Riboswitches are metabolite-sensing gene control elements typically located in the non-coding portions of messenger RNAs. Twelve structural classes of riboswitches in bacteria have been characterized to date that sense small organic compounds, including coenzymes, amino acids, and nucleotide bases (Mandal and Breaker, 2004; Soukup and Soukup, 2004; Winkler and Breaker, 2005; Fuchs et al., 2006; Roth et al., 2007) or magnesium ions (Cromie et al., 2006). In most instances, riboswitches can be divided into aptamer and expression platform regions that represent two functionally distinct but usually physically overlapping domains responsible for ligand binding and gene control, respectively.

The complexity of the structures formed by aptamers and their mechanisms of ligand recognition are evident upon examination of the atomic-resolution models elucidated by x-ray crystallography for several riboswitch classes, including those that bind guanine and adenine (Batey et al., 2004; Serganov et al., 2004), S-adenosylmethionine (Montange and Batey, 2006), TPP (Edwards and Ferre-D'Amare, 2006; Serganov et al., 2006; Thore et al., 2006), and glucosamine-6-phosphate (Kline and Ferré-D'Amaré, 2006; Cochrane et al., 2007). The nucleotide sequences of the ligand-binding core and supporting architectures of each aptamer class are highly conserved between different species as a result of their need to form a precise receptor for a specific ligand using only four nucleotide types. In contrast, the expression platforms for riboswitches can vary considerably between species, or even between multiple representatives of a riboswitch class in a single organism.

The high level of aptamer conservation allows researchers to employ bioinformatics methods to identify new riboswitch candidates (e.g. Grundy and Henkin, 1998; Gelfand et al., 1999; Barrick et al., 2004; Corbino et al., 2005; Weinberg et al., 2007) and to determine the distribution of known riboswitch classes in various organisms (e.g. Rodionov et al., 2002; Vitreschak et al., 2003; Nahvi et al., 2004; Abreu-Goodger and Merino, 2005). To date, these searches have revealed that only members of the TPP-sensing riboswitch class are present in all three domains of life (Sudarsan et al., 2003). In eukaryotes, TPP aptamers were found in thiamin metabolic genes from plants and filamentous fungi, but the mechanism of riboswitch function remained speculative (Kubodera et al., 2003; Sudarsan et al., 2003). In the fungus Neurospora crassa, a TPP aptamer resides in an intron within the 5′ region of NMT1 mRNA and recently it has been shown that TPP binding by the aptamer regulates NMT1 gene expression by controlling alternative splicing (Cheah et al., 2007). Specifically, TPP binding by the riboswitch prevents removal of intron sequences carrying upstream open reading frames (uORFs that preclude expression of the main ORF.

Herein, it is reported that TPP riboswitches are present in a variety of plant species where they reside in the 3′ UTR of the thiamin metabolic gene THIC. Formation of THIC transcripts with alternative 3′ UTR lengths is dependent on riboswitch function and mediates feedback regulation of THIC expression in response to changes in cellular TPP levels. The data indicate that 3′ UTR length correlates with transcript stability, thereby establishing a basis for gene control by alternative 3′ end processing. A detailed mechanism for TPP riboswitch function in plants is presented, which includes aptamer mediated control of splicing and differential 3′ end processing of THIC mRNAs. This study further reveals the versatility of riboswitch control in organisms from different domains of life and expands our knowledge on previously unknown aspects of eukaryotic gene regulation.

1. Results and Discussion

i. TPP Aptamers are Widely Distributed in Plant Species

The presence of highly conserved TPP-binding aptamers in the 3′ UTRs of the THIC genes from the plant species Arabidopsis thaliana, Oryza sativa and Poa secunda had been reported previously (Sudarsan et al., 2003). The collection of plant TPP aptamer representatives was expanded by sequencing THIC genes from additional plant species and by conducting database searches for nucleotide sequences that conform to the TPP aptamer consensus. After cDNA sequences were obtained, the corresponding regions from genomic DNAs of each species were cloned and sequenced (see Experimental Procedures for details), thus providing the sequences of both the initial and the processed mRNA molecules.

An alignment of all available TPP aptamer sequences from plants reveals a high level of conservation of nucleotide sequence and a secondary structure consisting of stems P1 through P5 (FIG. 1A). The major differences between eukaryotic TPP riboswitch aptamers from plants (FIG. 1B) and filamentous fungi (Cheah et al., 2007) compared to their bacterial and archaeal counterparts (FIG. 1C) (Winkler et al., 2002; Rodionov et al. 2002) are the consistent absence of a P3a stem frequently present in bacterial representatives and the variable length of the P3 stem in eukaryotes. Neither region is involved in TPP binding (Edwards and Ferre-D'Amare, 2006; Serganov et al., 2006; Thore et al., 2006; Cheah et al., 2007) and therefore these differences should not affect ligand binding specificity.

The TPP aptamer is found in the 3′ UTR of all known THIC examples from monocots, dicots and the conifer Pinus taeda. Interestingly, in the moss Physcomitrella patens, the TPP aptamer is present in the 3′ UTR of THIC (Ppa1), and also resides in the 3′ region of two genes that are homologous to the thiamin biosynthetic gene THI4 (Ppa2, Ppa3). This latter observation, and the observation that fungi also have TPP aptamers associated with multiple different genes (Cheah et al., 2007), indicates that eukaryotes likely use variants of the same riboswitch class to control multiple genes in response to changing concentrations of a key metabolite.

A striking characteristic of TPP aptamers from plants is the high level of nucleotide sequence conservation. Approximately 80% of the nucleotides (excluding the P3 stem) are conserved in all plant examples. In contrast, less than 40% are conserved in filamentous fungi. Most differences among plant TPP aptamers are found in the P3 stem, which varies both in length and sequence. Also, the length of the P3 stem varies between TPP aptamer representatives in the same species, as is observed in P. patens (FIG. 1A). The presence of both an extended P3 stem in THIC and very short P3 stems in THI4 suggests that there is no species-specific requirement for this component of the aptamer.

ii. THIC 3′ UTRs Vary in Length and Sequence

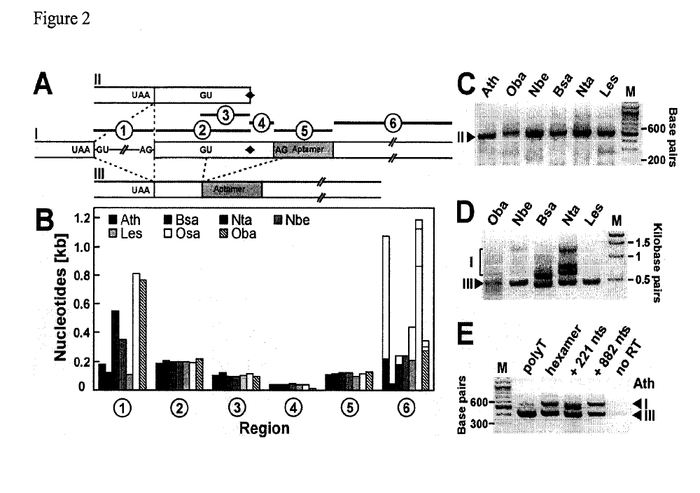

The nucleotide sequences of the 3′ regions of THIC mRNAs cloned from six plant species, or obtained from GenBank (O. sativa) were analyzed (FIG. 8; see also Experimental Procedures for details). Interestingly, the genomic organization of the 3′ region of THIC genes is conserved among these seven species, and the formation of three major types of processed RNA transcripts with varying 3′ UTR lengths is always observed (FIG. 2A). The stop codon for the THIC ORF is commonly followed by an intron that is typically spliced in all three RNA types. Type I (THIC-I) RNAs carry the complete aptamer and can extend to a variable length at its 3′ end. Type III (THIC-III) RNAs correspond to type I after the splicing of another intron that removes a portion of the TPP aptamer, whereas type II (THIC-II) RNAs terminate upstream of the aptamer.

Quantitation of the lengths of various regions (designated 1 through 6) within the THIC 3′ UTRs of these species reveals that some regions (2 through 5) exhibit considerable conservation of the numbers of nucleotides bridging key features within the UTR (FIG. 2B). In contrast, the length of the first intron (region 1) and the length of the 3′-most portion of THIC-I and THIC-III (region 6) are highly variable. For example, THIC-I and THIC-III can extend by more than 1 kb at their 3′ ends. The conservation of the distances between certain 3′ UTR features might be important for TPP-mediated gene regulation.

Reverse transcription and polymerase chain reaction (RT-PCR) was used to quantify the amounts of THIC transcript types. RT-PCR using a polyT primer and a primer specific for the THIC ORF (amplifies all THIC transcript types) results predominantly in amplification of THIC-II (FIG. 2C). This demonstrates that the short transcript form is most abundant in all species examined. Northern blot analysis with a probe that binds to the coding region of the THIC mRNA also results in one major signal corresponding to the size of THIC-II from A. thaliana (see further discussion below).

THIC-I and THIC-III were detected by RT-PCR using reverse primers that are specific for the extended 3′ region, and that do not recognize THIC-II RNAs (FIG. 2D). The lowest PCR product band for each species corresponds to THIC-III, whereas additional bands represent products derived from THIC-I that still retain one or both introns of the 3′ UTR, or represent minor splicing variants. Northern blot analysis using a probe specific for the 3′ UTR of THIC-I and THIC-III from A. thaliana confirmed that these transcript types are present in low copy number (see further discussion below) and also revealed heterogeneity of transcript length.

To assess whether 3′ end processing differs for the various transcript types in A. thaliana, RT-PCR was conducted using primers that permit amplification of specific regions of the transcripts. cDNAs generated either with polyT or random hexamer primers did not show a difference for amplification of THIC-II (data not shown) and THIC-III (FIG. 2E). However, the relative abundance of the THIC-I PCR product was strongly increased after amplification from cDNAs generated with random hexamer primers compared to polyT-derived cDNAs (FIG. 2E). This indicates that most THIC-I RNAs are not polyadenylated and therefore represent unprocessed THIC precursor transcripts. Also, cDNAs generated with primers binding far downstream of the aptamer sequence yielded PCR amplification products (FIG. 2E), indicating that THIC-I and THIC-III can extend more than 1 kb downstream of the annotated end of THIC in A. thaliana. Comparable THIC mRNAs with very long 3′ UTRs were also observed for O. sativa according to full length cDNA annotations in GenBank (AK068703, AK065235, AK120238). The formation of mRNAs with long 3′ UTRs is indicative of impairments in 3′ end processing and transcription termination.

iii. Thiamin Affects THIC Transcript Levels

The amount of THIC transcripts was established by using quantitative RT-PCR (qRT-PCR) to address whether transcript levels respond to increased thiamin concentrations. A. thaliana seedlings were supplemented with various amounts of thiamin and the different THIC transcript types were detected using specific primer combinations. The primer combination amplifying THIC-II also can bind to a subset of THIC-I RNAs that have undergone splicing of the first 3′ UTR intron. However, the contribution of the latter amplification product is minor because THIC-I transcripts are far less abundant and are almost undetectable when cDNAs are generated with polyT primers (FIG. 2E).

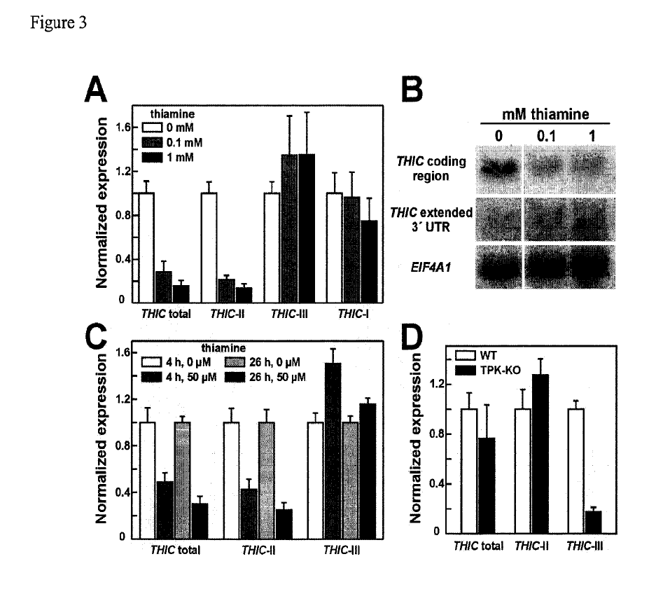

After growing seedlings on medium containing 1 mM thiamin, the total amount of THIC transcripts decreases to approximately 20% of that measured when seedlings are grown without thiamin supplementation (FIG. 3A). THIC-II transcripts exhibit an equivalent reduction, but both THIC-I and THIC-III transcripts show little or no change in copy number. Northern blot analysis of the same samples was used to confirm that THIC-II levels decrease and that and the relatively unchanging amounts of THIC-I and THIC-III RNA levels remain relatively unchanged (FIG. 3B).

The time interval in which thiamin-mediated changes in transcript levels occurs was assessed by performing qRT-PCR of THIC transcripts at several time points after spraying A. thaliana seedlings with a thiamin solution (FIG. 3C). Four hours after thiamin application, total THIC RNA and THIC-II amounts were reduced to 50% of that measured in the absence of added thiamin. After 26 h, these levels were decreased even further. Interestingly, the modest increase in THIC-III observed in this analysis when thiamin is added to the medium (FIG. 3A) is more pronounced in the early phase of the response. Because the different transcript types show an opposite response to thiamin treatment, the control mechanism most likely involves RNA processing, and it is unlikely that the feedback mechanism acts at the level of promoter regulation. Indeed, expression of a reporter gene driven by the THIC promoter from A. thaliana in transgenic lines was not altered after thiamin supplementation (FIG. 9).

Most of the thiamin taken up by cells is expected to be converted to TPP by successive phosphorylation reactions to yield concentrations of this coenzyme that are much higher than the concentration of the unphosphorylated vitamin (Ajjawi et al., in prep). Therefore, the observed reduction in total THIC RNA levels most probably reflects a riboswitch-mediated response to increased TPP concentration, given that TPP binding to plant aptamers is known to occur (Sudarsan et al., 2003; Thore et al., 2006). In this case, the opposite effect should occur when the TPP concentration decreases relative to that present in plants grown on medium without thiamin supplementation (assuming that the dynamic range for the riboswitch spans this TPP concentration range).

This was tested by comparing THIC expression in wild-type (WT) A. thaliana plants versus those carrying a double knockout of thiamin pyrophosphokinase (TPK). These mutants are deficient in both TPK isoforms present in A. thaliana and therefore cannot convert thiamin to TPP (Ajjawi et al., in prep). It has been shown that TPK double knockout (TPK-KO) plants largely deplete the TPP stored in seeds within two weeks of germination, and that the plants depend on TPP supplementation to complete their life cycle (Ajjawi et al., in prep). As predicted, qRT-PCR analysis of THIC RNAs from 12 day-old TPK-KO seedlings reveals an increase in the amount of THIC-II and a pronounced reduction of THIC-III compared to WT (FIG. 3D).

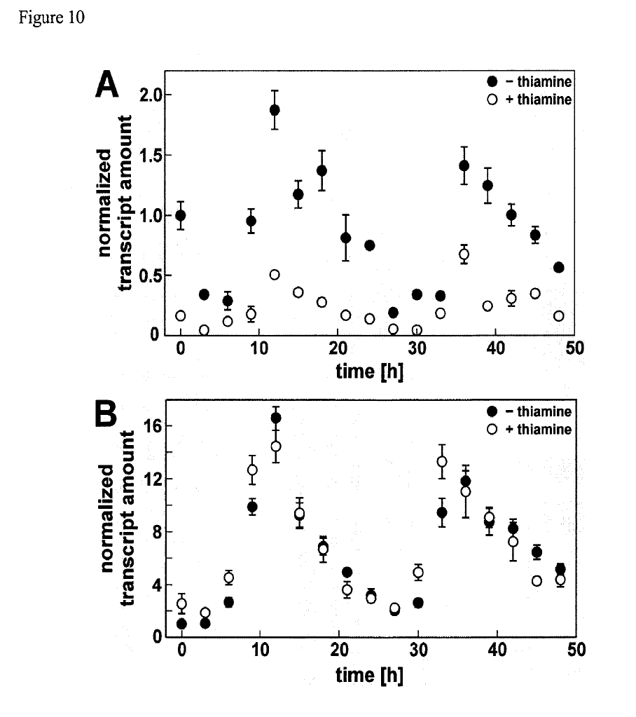

It is also notable that THIC expression in seedlings follows a circadian rhythm that is retained after transferring plants from a typical day-night cycle to continuous light, and this rhythm is not affected by thiamin treatment (FIG. 10). For both total THIC RNAs and THIC-III, the same rhythm phase was observed; demonstrating that riboswitch mediated feedback control does not affect the circadian rhythm of THIC expression.

iv. 3′ UTR Length Defines Gene Expression Levels

The presence of different THIC RNA types and their changes in abundance in response to varying thiamin levels suggest that the TPP aptamer might control RNA processing and that transcripts with different 3′ UTRs might be differentially expressed. It has been shown previously that the full-length aptamer from A. thaliana binds TPP with an apparent dissociation constant (K_{D}) of ˜50 nM (Sudarsan et al., 2003) and that its tertiary structure (Thore et al., 2006) is similar to that of bacterial TPP aptamers (Edwards and Ferre-D'Amare, 2006; Serganov et al., 2006). The precursor RNA, THIC-I, carries the complete aptamer and therefore it is expected to bind TPP.

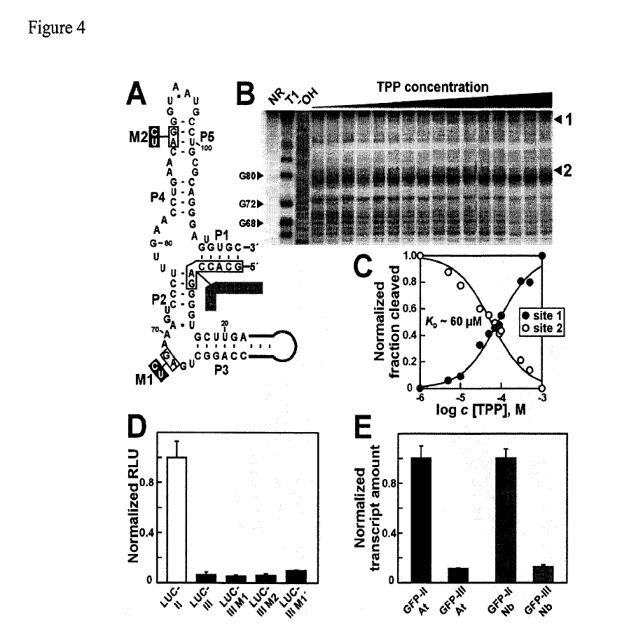

In contrast, THIC-III includes most of the consensus TPP aptamer sequence, but the first seven nucleotides at the 5′ end are removed due to splicing of the second intron in the 3′ UTR, and are replaced with different nucleotides (FIG. 4A, grey shaded sequence). In-line probing (Soukup and Breaker, 1999) was used to determine whether this altered aptamer retains TPP binding activity. This assay has been used previously to reveal structural changes in TPP aptamers (Sudarsan et al., 2003; Winkler et al., 2002) by monitoring altered patterns of spontaneous RNA degradation upon metabolite binding. The apparent K_{D} of the altered aptamer for TPP is ˜60 μM (FIGS. 4B and 4C), which is a loss of more than three orders of magnitude in ligand-binding affinity. Furthermore, thiamin does not bind to the altered aptamer (data not shown), and it is unlikely that other thiamin derivatives could be bound by this aptamer because the region of the aptamer that is exchanged upon splicing is not directly involved in ligand recognition (Edwards and Ferre-D'Amare, 2006; Serganov et al., 2006; Thore et al., 2006). These findings indicate that, once splicing of the second intron of the 3′ UTR occurs, the remainder of the TPP aptamer in THIC-III is no longer functional.

To assess possible effects of the two major THIC 3′ UTR forms on gene expression, the 3′ UTR sequences from THIC-II (188 nts) and THIC-III (408 nts) from A. thaliana were fused to the coding region of luciferase (LUC), and these constructs were expressed in plants under control of constitutive promoter and terminator elements. THIC-III can extend to a variable length at the 3′ end, but the most abundant shortest version (corresponding to GenBank entry NM179804) was used for the expression analyses. A fusion construct containing the 3′ UTR from THIC-III resulted in only ˜10% of the LUC activity compared to a construct carrying the 3′ UTR from THIC-II (FIG. 4D). The possible involvement of the altered TPP aptamer in the type III construct was ruled out by introducing mutations M1 and M2 that completely abolish TPP binding, but do not derepress LUC expression. Also, using the reverse complement sequence of the THIC-III 3′ UTR sequence did not change LUC activity significantly. These data indicate that the extended length, and not the altered TPP aptamer, plays a role in the repression of constructs containing the 3′ UTR from type III RNAs. Equivalent results were obtained with constructs containing the reporter gene EGFP in place of LUC, and coexpression of the silencing suppressor P19 excluded the possibility that the observed differences are due to silencing effects in the reporter system (FIG. 11).

It was also assessed whether differences in reporter activity are also reflected in transcript amounts. Using qRT-PCR, the relative amounts of reporter transcripts containing the 3′ UTRs from THIC-II or THIC-III from either A. thaliana or N. benthamiana were determined (FIG. 4E). Constructs carrying the long 3′ UTR of type III RNAs from both species were present in lower abundance compared to those that carried the short type II 3′ UTR. Since all reporter constructs were expressed under control of a constitutive promoter and terminator, transcription initiation and termination should be the same for all constructs.

The findings suggest that long 3′ UTRs cause increased transcript turnover. Thus, riboswitch-mediated redirection of RNA processing to favor the production of mRNAs with extended 3′ UTRs should reduce THIC expression. This hypothesis is consistent with previous studies showing that long 3′ UTRs induce nonsense-mediated decay (NMD) in yeast (Muhlrad and Parker, 1999) and plants (Kertesz et al., 2006). In the latter study, a reduction in the abundance of mRNAs with 3′ UTR lengths above 200 nts was observed, as was a correlation between 3′ UTR length and NMD efficiency. Furthermore, the results suggest that this mechanism is involved not only in mRNA quality surveillance (Fasken and Corbett, 2005), but also plays a role in regulation of gene expression in plants.

v. Riboswitch Function in Thiamin Feedback Response

Although the splice-modified TPP aptamer does not affect expression of processed THIC-III RNAs, the unaltered TPP aptamer might be part of a riboswitch that alone can regulate the processing of THIC mRNA transcripts to yield RNAs with different 3′UTR lengths. This was explored by analyzing the expression of reporter constructs containing EGFP fused with the complete genomic 3′ region of THIC (˜2.2 kb downstream of the stop codon) in stably transformed A. thaliana plants. Thiamin application resulted in decreased EGFP fluorescence in leaves from the rosette stage (FIGS. 5A and 5B). Using qRT-PCR analysis, it was found that the amounts of both EGFP and endogenous THIC transcripts were reduced to approximately 20% of control levels after thiamin feeding (FIG. 5C), which is similar to that observed for A. thaliana seedlings (FIG. 3).

The 3′ UTR sequences of EGFP fusion and THIC transcripts from the transformants were amplified by RT-PCR (FIGS. 5D and 5E), cloned and sequenced. Sequence analyses confirmed the formation of equivalent transcript processing types for EGFP and THIC (see also FIG. 2). The difference in total transcript amount of THIC and EGFP can be explained by the use of a strong promoter for control of the transgene. Because the thiamin responses and processed RNAs between the reporter gene construct and THIC were identical, it was concluded that no additional sequences upstream of the region fused to EGFP are involved in the gene control mechanism.

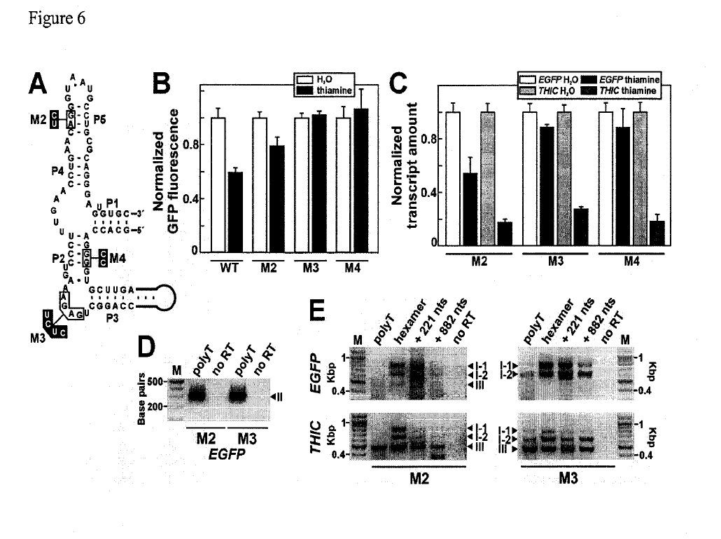

To determine whether the effects of thiamin regulation are mediated through a TPP riboswitch, mutations M2, M3 and M4 were introduced into the aptamer (FIG. 6A) that reduces TPP binding affinity. M2 and M4 mutations interfere with formation of stems P5 and P2 of the TPP aptamer, respectively. With M3, three nucleotides that are known to be involved in direct interactions with the pyrimidine moiety of TPP (Edwards and Ferre-D'Amare, 2006; Serganov et al., 2006; Thore et al., 2006) are mutated. 3′ regions of THIC carrying these variants were fused to EGFP and stably transformed into A. thaliana plants.

As expected, plants containing reporter gene constructs carrying the mutant aptamers exhibit either reduced (M2) or a complete loss (M3 and M4) of responsiveness to thiamin application compared to the WT construct (FIG. 6B). These findings were confirmed by measuring the relative levels of transcripts using qRT-PCR (FIG. 6C). In addition, a reporter construct variant of M4 containing compensatory mutations that restore formation of P2 (and thereby restore TPP binding) exhibits activity similar to WT (data not shown). These results indicate that TPP binding by the aptamer is essential for mediating the response to changing TPP levels in the cell. However, the modest thiamin responsiveness exhibited by the M2 construct suggests this mutant might affect riboswitch function other than just by diminishing the affinity of the aptamer for TPP (see further discussion below).

RT-PCR analyses of 3′ ends of the mRNAs generated from the EGFP-riboswitch fusions reveal that the mutant constructs maintain a high level of expression of type II RNAs (FIG. 6D), as is typical of WT constructs. However, two major differences in type I and III RNAs between mutant and WT riboswitches are evident. First, the amount of type III RNA is substantially reduced in the M2 construct and was not detectable from the M3 construct (FIG. 6E). Second, a considerable decrease of transcripts extending far downstream of the aptamer was observed for both mutants (FIG. 6E, 882 nts lane, see also WT in FIG. 5E). These results reveal that proper riboswitch function is required for the production of mRNAs with different 3′ UTR sequences and lengths, which leads to thiamin-dependent down regulation of gene expression.

vi. Mechanism of Riboswitch Function

In-line probing was used to explore how the TPP riboswitch might control 3′ end processing of THIC mRNAs from A. thaliana. An aptamer construct that included 14 nts upstream of the 5′ splice site for the second 3′ UTR intron exhibited TPP-dependent structural modulation of 8 nts immediately upstream of the splice site (FIG. 7A). Specifically, TPP addition causes an increase in structural flexibility of the nucleotides near the 5′ splice site. Thus, ligand binding could increase accessibility of the splice site to the spliceosome, thereby permitting the removal of this intron.

Base-pairing potential between the sequences of the modulating 5′ splice site nucleotides and the aptamer nucleotides of THIC genes from several plant species were searched for. In all species examined, the 5′ side of the P4-P5 stems are complementary to the nucleotides immediately upstream (and sometimes inclusive) of the 5′ splice site (FIG. 7B). This conservation of base-pairing potential suggests that the riboswitch controls splicing by the mutually-exclusive formation of structures that either mask the 5′ splice site under low TPP concentrations, or expose the splice site under high TPP concentrations (FIG. 7C).

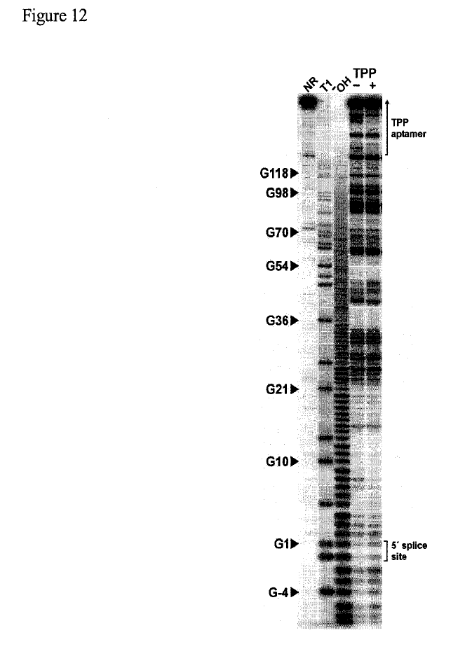

This model is consistent with the in vitro and in vivo data generated in the current study, including the partial thiamin responsiveness observed with the M2 variant. M2 carries two mutations that disrupt the P5 stem of the aptamer (FIG. 6A), which should weaken its interaction with TPP and disrupt thiamin responsiveness. However, these mutations also weaken base pairing with the 5′ splice site region, which might allow TPP binding to compete effectively with this alternative pairing, despite the expected reduction in TPP affinity. One remarkable feature of plant TPP riboswitches is that the 5′ splice sites under riboswitch control are located more than 200 nts upstream of the complementary regions in the TPP aptamers (FIG. 2A). The complex structural organization of the sequences between the complementary regions (FIG. 12) might be important to bring these sites close together in space to facilitate their interaction, which might also explain the conservation of lengths between features of THIC UTRs from various plants (FIG. 2A).

Interestingly, TPP riboswitches also control alternative splicing of the NMT1 genes of fungi in part by forming ligand-modulated base pairing between nucleotides near a 5′ splice site and the P4-P5 region of an unoccupied TPP aptamer (Cheah et al., 2007). In contrast to these eukaryotic examples, bacteria typically use nucleotides in P1 stems to interface with expression platforms located downstream of the aptamer (Sudarsan et al., 2005; Winkler et al., 2002). Given the substantial changes in the structure of TPP aptamers upon ligand binding, it is surprising that only a portion of the P1 and P4-P5 stems are used to control expression platform function in the TPP riboswitches studied to date. One reason for this might be the need for preorganization of certain aptamer substructures to facilitate rapid ligand sensing.

vii. Model for TPP Riboswitch Function in Plants

Earlier studies indicated that transcription terminators similar to those found in bacteria might also exist in eukaryotes (Proudfoot, 1989). Interestingly, a poly-uridine tract immediately follows the aptamer in all known TPP riboswitch examples in plants (see FIG. 8), and this element might be involved in polymerase release analogous to intrinsic transcription terminators in bacteria (Yarnell and Roberts, 1999; Gusarov and Nudler, 1999). However, no RNA transcripts were identified that are consistent with products expected if eubacteria-like transcription termination were occurring.

A different model is proposed for TPP riboswitch regulation in plants involving the metabolite-mediated control of splicing and alternative 3′ end processing of mRNA transcripts (FIG. 7C). When TPP concentration in cells is low, the aptamer interacts with the 5′ splice site and prevents splicing. This intron carries a major processing site that permits transcript cleavage and polyadenylation. Processing from this site produces THIC-II transcripts that carry short 3′ UTRs and that yield high expression of the THIC gene.

When TPP concentrations are high, TPP binding to the aptamer prevents pairing to the 5′ splice site. As a result, the 5′ splice site becomes accessible and is used in a splicing event that removes the major processing site. Transcription subsequently extends up to 1 kb and the use of processing sites located downstream gives rise to THIC-III RNAs that carry much longer 3′ UTRs. The long 3′ UTRs cause increased transcript degradation and THIC expression is reduced. Previous studies have shown that extended transcription occurs in the absence of transcript processing, thus revealing the interconnectivity of these processes (Buratowski, 2005; Proudfoot, 2004; Proudfoot et al., 2002).

Two different models have been proposed for how transcript processing and transcription termination in eukaryotes are coupled. The “antiterminator” model suggests that transcription of the termination site results in a conformational change of the transcription complex that leads to termination (Logan et al., 1987). In contrast, the “torpedo” model indicates that the cleavage event is the prerequisite for transcription termination (Connelly and Manley, 1988). Other transcription termination mechanisms also might exist. Recent reports indicate that additional cotranscriptional cleavage events, which occur downstream of the processing site in some genes, might play a role in controlling termination (Dye and Proudfoot, 2001; Proudfoot, 2004; Proudfoot et al., 2002). Furthermore, it has been demonstrated that autocatalytic RNA cleavage can be involved in transcript 3′ end formation (Teixeira et al., 2004; Vader et al., 1999). Although other mechanisms cannot be ruled out, the observation that THIC TPP riboswitches control splicing and processing site access to regulate transcription termination is consistent with the torpedo model.

viii. Conclusions

The findings reveal a mechanism for how TPP-sensing riboswitches can control gene expression in plants and how feedback control maintains TPP levels. In addition, this study further expands the known diversity of mechanisms that riboswitches use to regulate gene expression. The TPP riboswitch in A. thaliana harnesses metabolite binding to control RNA splicing, which determines alternative 3′ end processing fate, which ultimately defines the stability of mRNAs. The extensive conservation of sequences, structural elements, and spacing between key 3′ UTR features within the THIC genes of various plants indicates that this riboswitch mechanism is maintained in diverse plant species. Independent of riboswitch-mediated regulation, the potential for the control of genes by regulating alternative 3′ end processing appears to be large, and therefore this general mechanism might be far more widespread in eukaryotes.

Preliminary findings indicate that THIC overexpression causes detrimental effects in plants. This highlights the importance of control of thiamin production in plants, which might also be linked to its recently discovered role as an activator of plant disease resistance (Ahn et al., 2005; Ahn et al., 2007; Wang et al., 2006). A deeper understanding of the control of thiamin biosynthesis in plants might also be useful for metabolic engineering purposes, as plants serve as primary nutritional source of vitamin B₁.

The unique location of TPP riboswitches in the 3′ regions of plant genes compared to their locations in fungi and bacteria might reflect adaptations to specific regulatory needs of different organisms. Nearly all known riboswitches reside in the 5′ UTRs of bacteria (Mandal and Breaker, 2004; Soukup and Soukup, 2004; Winkler and Breaker, 2005) or in introns of 5′ UTRs or coding regions of fungi (Cheah et al., 2007) and often can suppress gene expression almost completely. However, a more subtle level of riboswitch regulation is observed in plants. Although plants can take up thiamin efficiently, most of the demand must be supplied by endogenous synthesis. In contrast to the autotrophic lifestyle of plants, fungi and bacteria sometimes grow under rich conditions that allow them to satisfy their entire requirements for compounds like thiamin by import, thus providing some rationale for different extents of regulation found in organisms from different domains of life.

2. Experimental Procedures

i. Plants and Plant Tissues

Arabidopsis thaliana ecotype Columbia-0 plants were grown with soil at 23° C. in a growth chamber under 16/8 h (light/dark) photoperiod with 60% humidity unless otherwise stated. For seedling experiments, plants were grown on basal MS medium (Murashige and Skoog, 1962) supplemented with 2% sucrose and varying concentrations of thiamin and under continuous light unless otherwise specified. N. benthamiana plants for leaf infiltration assays were grown on soil for 3 to 5 weeks under continuous light. Plant material from other species was derived from seedlings grown from commercially available seeds.

ii. RNA Isolation and RT-PCR Analyses

Total RNA was extracted from frozen plant tissues using the RNeasy Plant Mini Kit (QIAGEN) following the manufacturer's instructions. 2-5 μg of total RNA were subjected to DNase treatment and subsequently reverse transcribed using SuperScript™ II Reverse Transcriptase (Invitrogen) according to the manufacturer's instructions. For cDNA generation, gene specific primers or (if not otherwise specified) a polyT primer (DNA1) were used. cDNAs were used as templates for PCR amplification of THIC and EGFP reporter transcripts. All products obtained were cloned into TOPO-TA cloning vector (Invitrogen) and analyzed by sequencing (HHMI Keck Foundation Biotechnology Resource Center at Yale University).

qRT-PCR) was performed using the Applied Biosystems 7500 Real-Time PCR System and Power SYBR Green Master Mix (Applied Biosystems). Serial dilutions of the templates were conducted to determine primer efficiencies for all primer combinations. Each reaction was performed in triplicate, and the amplification products were examined by agarose gel electrophoresis and melting curve analysis. Data were analyzed using the relative standard curve method and the abundance of target transcripts was normalized to reference transcripts reported previously (Czechowski et al., 2005) from genes AT1G13320 (PP2A catalytic subunit), AT5G60390 (EF-1α), and At1G13440 (GAPDH).

iii. Amplification of THIC Transcripts and Genomic Sequences from Plants

3′ UTRs from THIC-II RNAs were cloned by using RT-PCR with a polyT primer and a degenerate primer that targets a conserved portion of the coding sequence near the stop codon. For THIC-III transcripts, 3′ UTRs were amplified in two fragments from polyT generated cDNA using specific primer combinations. The 5′ portion of each 3′ UTR was PCR amplified using a degenerate primer targeting the coding region and a primer that targets the TPP aptamer. The 3′ portion of each 3′ UTR was obtained by using a primer targeting the aptamer and a polyT primer. PCR products were cloned (TOPO-TA) and several independent clones were sequenced. The combined sequence information was used to design primer pairs for amplification of the corresponding genomic sequences. Genomic DNA was isolated using Plant DNAzo1 Reagent (GibcoBRL) according to the manufacturer's instructions and the resulting PCR products were cloned and sequenced.

iv. Northern Blot Analysis

Transcripts from A. thaliana seedlings were analyzed by Northern blot analysis as described previously (Newman et al., 1993). Probes were specific against regions in the coding region of THIC, the extended 3′ UTR of THIC types I and III RNAs, or the control transcript EIF4A1.

v. Agrobacterium-Mediated Leaf Infiltration Assay

For transient gene expression analysis, N. benthamiana leaves were transformed by a leaf infiltration assay as described by (Cazzonelli and Velten, 2006). Agrobacterium lines harboring the various reporter constructs were grown over night in LB medium, centrifuged, and the pelleted cells were resuspended in H₂O. OD₆₀₀ was adjusted to the same value (−0.8) for cells harboring the different constructs and Agrobacteria were mixed in equal amounts for cotransformation of constructs. Either luciferases from firefly (Photinus pyralis) or sea pansy (Renilla reniformis), or the fluorescent proteins EGFP and DsRed2, were used as reporter proteins.

Luciferase activity was measured using a dual-luciferase reporter assay system (Promega). Leaf material was typically harvested 60 h after infiltration and frozen in liquid nitrogen (˜100 mg per sample). After grinding, 100 μl 1× Passive Lysis Buffer (Promega) was added and mixed with the sample vigorously. Samples were incubated for 1 h on ice followed by centrifugation for 20 min at 13,000 g. The resulting supernatant was diluted 1:40 and luciferase activity was measured by subsequent addition of the dual luciferase assay buffers in a plate-reading luminometer (Wallac). Activity of firefly luciferase was normalized to the activity of coexpressed luciferase from sea pansy (or vice versa) or relative to total protein amount determined by Bradford Protein Assay (BioRad).

For fluorescence quantitation, leaves were scanned at several time points after infiltration using a Typhoon Trio+ laser scanner (Amersham Biosciences). Settings for EGFP were excitation at 488 nm and detection at 520 nm BP 40. DsRed2 was excited at 532 nm and detected at 580 nm BP 30. Leaves were not significantly damaged by scanning and were incubated with the petioles in H₂O after excision.

vi. Stable Transformation of A. thaliana by Floral Dip Method

A. thaliana was transformed by a floral dip method described previously (Clough and Bent, 1998). After transformation, seeds were grown under sterile conditions on medium containing 50 μg ml⁻¹ kanamycin to select for transformants, and 200 μg ml⁻¹ cefotaxime to prevent bacterial growth. Surviving plants were transferred after 2-3 weeks to soil and expression of the transgene was determined after further growth.

vii. Cloning of DNA Constructs

All reporter constructs were based on the plasmid pBinAR (Höfgen and Willmitzer, 1992), which contains the constitutive CaMV ³⁵S promoter. The coding sequence of luciferase from Photinus pyralis (firefly) was amplified with primers DNA44 and DNA45 and, after restriction with BamHI and SalI, was cloned into appropriate sites of pBinAR to obtain pBinARFLUC. In pBinARFLUC, the peroxisomal targeting sequence at the C-terminus of luciferase was replaced by the amino acid sequence “IAV” to prevent peroxisome localization. To prepare pBinARRiLUC, an intron containing version of luciferase from the sea pansy Renilla reniformis (Cazzonelli and Velten, 2003) was amplified with primers DNA46 and DNA47 and, after restriction, cloned into BamHI/SalI sites of pBinAR. To prepare plasmids containing fluorescent proteins as reporters, the coding sequences of EGFP and DsRed2 were amplified with primers DNA48/49 and DNA 50/51, respectively. After restriction with BamHI/SalI, products were cloned into appropriate sites of pBinAR.

3′ UTR sequences from A. thaliana THIC type II and III RNAs were amplified with primers DNA2/52 and DNA2/3, respectively and cloned into the SalI site of the pBinAR reporter plasmids. For cloning of corresponding constructs based on THIC sequences from N. benthamiana, 3′ UTRs from type II and III RNAs were amplified with primers DNA 53/54 and DNA53/55, respectively. Sequences and orientation of THIC 3′ UTRs in reporter fusion constructs were confirmed by sequencing.

For generation of the aptamer mutants M1 and M2 (in the context of type III RNAs), the wild-type 3′ UTR sequence of THIC-III from A. thaliana was amplified with DNA2 and DNA3, and cloned using a TOPO TA cloning kit (Invitrogen). PCR mutagenesis was performed on the THIC-III 3′ UTR in the TOPO TA vector and the nucleotide changes were confirmed by sequencing. Subsequently, the 3′ UTR sequences were released from the vector by restriction with SalI and cloned into the appropriate site of the reporter plasmid.

To prepare constructs containing the riboswitch in its genomic context, a fragment of 2242 by starting from the translational stop codon of THIC was amplified from A. thaliana genomic DNA with primers DNA60 and DNA61 and cloned into the TOPO TA vector. As pBinAR contains an Agrobacterium derived octopine synthase (OCS) terminator, that might interfere with riboswitch function, the OCS sequence was removed by restriction with SalI and HindIII and the vector religated using a linker consisting of two complementary oligonucleotides (DNA62, DNA63) with the appropriate restriction sites resulting in vector pBinAR-term. This vector without the terminator sequence was used for subsequent cloning. The coding sequence of EGFP was amplified with primers DNA48 and DNA49 and, after restriction with BamHI and SalI, was cloned into appropriate sites of pBinAR-term. In a second step, the genomic THIC fragment was released from the TOPO TA vector by SalI digestion and cloned into the SalI site of pBinAREGFP-term. Sequence and orientation of the THIC fragment were confirmed by sequencing. For generation of aptamer mutants M2, M3 and M4, PCR mutagenesis was performed on the TOPO TA plasmid containing the THIC 3′ fragment and, after sequence confirmation, the SalI fragment was cloned into the appropriate site of pBinAREGFP-term. Again, sequence and orientation of the THIC fragment were confirmed by sequencing.

viii. In-Line Probing of RNA

In-line probing assays were conducted essentially as described previously (Sudarsan et al., 2003; Winkler et al., 2002). The DNA template for in vitro transcription was obtained by PCR amplification from cDNA and a T7 promoter was introduced by inclusion in the forward primer. In vitro transcription, RNA purification by denaturing polyacrylamide gel electrophoresis (PAGE), and 5′ ³²P-labelling of the RNA were performed as described previously (Seetharaman et al., 2001). For in-line probing analysis, the labeled RNA was incubated at room temperature for 40 hours in 50 mM Tris-HCl (pH 8.3 at 23° C.), 20 mM MgCl₂, and 100 mM KCl in the absence or presence of varying concentrations of TPP. Cleavage products were resolved by denaturing 10% PAGE, visualized by PhosphorImager (GE Healthcare), and quantitated using ImageQuant software. The apparent K_{D} value, reflecting the concentration of TPP needed to half-maximally modulate RNA structure, was determined by plotting the normalized fraction of RNA cleaved versus the logarithm of TPP concentration.

TABLE 1
Sequences of DNA primers (SEQ ID NOs: 55-131)

RT-PCR analysis THIC from Arabidopsis

| | | |
|---|---|---|
| DNA1 | 5′-GCTGTCAACGATACGCTACGTAACGGCATGACAGTGTT | polyT |
| | TTTTTTTTTTTTTTTTTT | |
| DNA2 | 5′-AGCTGTCGACAAGGCAAATGTTTTAAACAAGACC | SalI; for 3′ UTR |
| DNA3 | 5′-AGCTGTCGACGGTGCAAATGCATTTTTATCAATC | SalI; rev +221 nt |
| DNA4 | 5′-CAGTCACAAAGCCTACGATCAA | rev +882 nt |
| DNA5 | 5′-CGGTGAAGTAGGTGGAGAAA | for, end of coding region |

RT-PCR analysis EGFP

| | | |
|---|---|---|
| DNA6 | 5′-CGGGATCACTCTCGGCATG | for |

RT-PCR analysis THIC from more plant species

| | | |
|---|---|---|
| DNA7 | 5′-GCACAYTTYTGCTCNATGTGYGG | for, end of coding region |
| DNA8 | 5′-GGTTCAAAGGGACTTTCTCAG | rev; conserved aptamer |
| | | region |
| DNA9 | 5′-CTGAGAAAGTCCCTTTGAACC | for; conserved aptamer |
| | | region |

Amplification of THIC 3′ genomic fragment

| | | |
|---|---|---|
| DNA10 | 5′-ACCGAAATTCTGCTCCATGAA | for; Bsa |
| DNA11 | 5′-AGCAGAAAAGCTTCATCTCC | rev; Bsa |
| DNA12 | 5′-GCCAAAGTTTTGTTCTATGAAAA | for; Nta |
| DNA13 | 5′-GCAGTGGTCAAAAATTGTACAC | rev; Nta |
| DNA14 | 5′-GCCAAAGTTTTGTTCTATGAAG | for; Nbe |
| DNA15 | 5′-GCAGTGGTCAAAAATTGTACAC | rev; Nbe |
| DNA16 | 5′-TCCTAAGTTTTGCTCCATGAAA | for; Les |
| DNA17 | 5′-CCAGATCTTAAATTCGTAATATT | rev; Les |
| DNA18 | 5′-TTGGCGGCGAAGAAGACG | for; Oba |
| DNAI9 | 5′-AAATCTTTAAGAGCCTTGTTTTTT | rev; Oba |

qRT-PCR analysis

| | | |
|---|---|---|
| DNA20 | 5′-ATGTGCAGGTGATGAATGAAGG | for; THIC total |
| DNA21 | 5′-GTAGAATGGTGCCTCGTTACACC | rev; THIC total |
| DNA22 | 5′-CTGCTCAGAAATAAAAGGCAAATG | for; THIC II |
| DNA23 | 5′-CTACTAAGCTTACCAACAGTTTGTGCC | rev; THIC II |
| DNA24 | 5′-GCACAAACTGTTGGGGTGC | for; THIC III |
| DNA25 | 5′-CATTACCCTGTTCAGGTTCAAAGG | rev; THIC III |
| DNA26 | 5′-AATACTTTTTTGTGTGATTTGGTTGG | for; THIC |
| DNA27 | 5′-AGCCTGGTCCCGGATAGC | rev; THIC I |
| DNA28 | 5′-GGTAATAACTGCATCTAAAGACAGAGTTCC | for; AT1G13320 |
| DNA29 | 5′-CCACAACCGCTTGGTCG | rev; AT1G13320 |
| DNA30 | 5′-GTGTCTACCGACTTTGGTCAAGC | for; At1G13440 |
| DNA31 | 5′-ACCCCATTCGTTGTCGTACC | rev; At1G13440 |
| DNA32 | 5′-CTGCTGCCCGACAACCA | for; EGFP |
| DNA33 | 5′-GAACTCCAGCAGGACCATGTG | rev; EGFP |
| DNA34 | 5′-AGACCCACAAGGCCCTGAA | for; DsRed2 |
| DNA35 | 5′-CAGCTGCACGGGCTTCTT | rev; DsRed2 |

Probes for RNA gel blot analysis

| | | |
|---|---|---|
| DNA36 | 5′-CAAGCGTTTGACCGGGA | for; coding region |
| DNA37 | 5′-ATGCGTCGACTTATTTCTGAGCAGCTTTGAC | rev; coding region |
| DNA38 | 5′-GGGTGCTTGAACCAGGA | for; extended 3′ UTR |
| DNA39 | 5′-AGCTGTCGACGGTGCAAATGCATTTTTATCAATC | rev; extended 3′ UTR |

in vitro transcription
TPP aptamer present in THIC transcript type III

| | | |
|---|---|---|
| DNA40 | 5′-TAATACGACTCACTATAGGCAAACTGTTGGGGTGCTTG | for; T7 promoter |
| DNA41 | 5′-CACACTCCCTGCGCAGGC | rev |

TPP aptamer with 5′ flank (nts-14-261 relative to 5′ splice site)

| | | |
|---|---|---|
| DNA42 | 5′-TAATACGACTCACTATAGGCACAAACTGTTGGTAA | or; T7 promoter |
| DNA43 | 5′-AAACTGCACACTCCCTG | |

Cloning of reporter constructs

| | | |
|---|---|---|
| DNA44 | 5′-AGCTGGATCCGCATTCCGGTACTGTTGG | for; BamHI |
| DNA45 | 5′-AGCTGTCGACTTATACGGCTATTCCGCCCTTCTTGGCC | rev; SalI |
| | TTTATG | |
| DNA46 | 5′-AGCTGGATCCATGACTTCGAAAGTTTATG | for; BamHI |
| DNA47 | 5′-AGCTGTCGACTTATTGTTCATTTTTGAGAAC | rev; SalI |
| DNA48 | 5′-AGCTGGATCCATGGTGAGCAAGGGCGAGGAG | for; BamHI |
| DNA49 | 5′-AGCTGTCGACTTACTTGTACAGCTCGTCCATGC | rev; SalI |
| DNA50 | 5′-AGCTGGATCCATGGCCTCCTCCGAGAAC | for; BamHI |
| DNA51 | 5′-AGCTGTCGACCTACAGGAACAGGTGGTG | rev; SalI |
| DNA52 | 5′-AGCTGTCGACATTGAAACATCAACTTAGATTGTC | rev; SalI |
| DNA53 | 5′-AGCTGTCGACAGGACTTCATAGATGGAAAA | for; SalI |
| DNA54 | 5′-AGCTGTCGACTAAAAAACGCGATTTCTTATTA | rev; SalI |
| DNA55 | 5′-AGCTGTCGACGCCCGAAATGTGCCCCG | rev; SalI |
| DNA56 | 5′-TCCGGGACCAGGCTGTCAAAGTCCCTTTGAAC | for; M1 |
| DNA57 | 5′-GTTCAAAGGGACTTTGACAGCCTGGTCCCGGA | rev; M1 |
| DNA58 | 5′-CCTTTGAACCTGAACTCGGTAATGCCTGCGC | for; M2 |
| DNA59 | 5′-GCGCAGGCATTACCGAGTTCAGGTTCAAAGG | rev; M2 |
| DNA60 | 5′-AGCTGTCGACAAGGTCAGTATGTTTAGACTGTTAG | for; SalI |
| DNA61 | 5′-AGCTGTCGACCTCTCCACCTAAACTCAGATTTTG | rev; SalI |
| DNA62 | 5′-AGCTGTCGACACCGGTGAGCTCACTAGTAAGCTTAGCT | for; SalI, HindI II |
| DNA63 | 5′-AGCTAAGCTTACTAGTGAGCTCACCGGTGTCGACAGCT | rev; HindI II, SalI |
| DNA64 | 5′-TCCGGGACCAGGCTCTCTAAGTCCCTTTGAAC | for; M3 |
| DNA65 | 5′-GTTCAAAGGGACTTAGAGAGCCTGGTCCCGGA | rev; M3 |
| DNA66 | 5′-GCACCAGCCGTGCTTGAAC | for; M4 |
| DNA67 | 5′-GTTCAAGCACGGCTGGTGC | rev; M4 |

THIC promoter-GUS expression analysis

| | | |
|---|---|---|
| DNA68 | 5′-CACCCTTCTCCTTCTAGTGAAT | for, THIC promoter |
| DNA69 | 5′-AGCTGGAGACAAACGAAA | rev, THIC promoter |
| DNA70 | 5′-ATGTGCAGGTGATGAATGAAG | for, qRT-PCR THIC |
| DNA71 | 5′-CAAAGGACCAAGGGTGTAGAA | rev, qRT-PCR THIC |
| DNA72 | 5′-TGGAGTGGTGTAACGAG | probe, qRT-PCR THIC |
| DNA73 | 5′-GCGT*CAATGTAATGTTCT | for, qRT-PCR GUS |
| DNA74 | 5′~TCTCTGCCGT*TTCCAAATC | rev, qRT-PCR GUS |
| DNA75 | 5′-GATGTGCTGTGCCTGAA | probe, qRT-PCR GUS |
| DNA76 | 5′-GAGCCCAAGTTTTTGAAGA | for, qRT-PCR eEF-1α |
| DNA77 | 5′-CTAACAGCGAAACGTCCCA | rev, qRT-PCR eEF-1α |
| DNA78 | 5′-CCCCAACCAAGCCCAT | probe, qRT-PCR eEF-1α |

“*” identifies nucleotides that were introduced to increase the efficiency of the combination of primers and probe in qRT-PCR. Forward and reverse primers are designated “for” and “rev”, respectively.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a riboswitch” includes a plurality of such riboswitches; reference to “the riboswitch” is a reference to one or more riboswitches and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

### REFERENCES

Abreu-Goodger, C., and Merino, E., (2005). RibEx: a web server for locating riboswitches and other conserved bacterial regulatory elements. Nucleic Acids Res. 33, W690-692.
Ahn, I. P., Kim, S., and Lee, Y. H. (2005). Vitamin B1 functions as an activator of plant disease resistance. Plant Physiol. 138, 1505-1515.
Barrick, J. E., Corbino, K. A., Winkler, W. C., Nahvi, A., Mandal, M., Collins, J., Lee, M., Roth, A., Sudarsan, N., Jona, I., et al. (2004). New RNA motifs suggest an expanded scope for riboswitches in bacterial genetic control. Proc. Natl. Acad. Sci. USA 101, 6421-6426.
Batey, R. T., Gilbert, S. D. & Montange R. K. Structure of a natural guanine-responsive riboswitch complexed with the metabolite hypoxanthine. Nature 432, 411-415 (2004).
Blencowe, B. J. Alternative splicing: new insights from global analyses. Cell 126, 37-47 (2006).
Borsuk, P., et al. L-Arginine influences the structure and function of arginase mRNA in Aspergillus nidulans. Biol. Chem. 388, 135-144 (2007).
Buratowski, S. (2005). Connections between mRNA 3′ end processing and transcription termination. Curr. Opin. Cell Biol. 17, 257-261.
Buratti, E. & Baralle, F. E. Influence of RNA secondary structure on the pre-mRNA splicing process. Mol. Cell. Biol. 24, 10505-10514 (2004).
Cazzonelli, C. I., and Velten, J. (2003). Construction and testing of an intron-containing luciferase reporter gene from Renilla reniformis. Plant Mol Biol Rep 21, 271-280.
Cazzonelli, C. I., and Velten, J. (2006). An in vivo, luciferase-based, Agrobacterium-infiltration assay system: implications for post-transcriptional gene silencing. Planta 224, 582-597.
Cheah, M. T., Wachter, A., Sudarsan, N., and Breaker, R. R. (2007). Control of alternative RNA splicing and gene expression by eukaryotic riboswitches. Nature (in press).
Clough, S. J., and Bent, A. F. (1998). Floral dip: a simplified method for Agrobacterium-mediated transformation of Arabidopsis thaliana. Plant J. 16, 735-743.
Cochrane, J. C., Lipchock, S. V., and Strobel, S. A. (2007). Structural investigation of the gimS ribozyme bound to its catalytic cofactor. Chem. Biol. 14, 97-105.
Colot, H. V., Loros, J. J. & Dunlap, J. C. Temperature-modulated alternative splicing and promoter use in the circadian clock gene frequency. Mol. Biol. Cell 16, 5563-5571 (2005).
Connelly, S., and Manley, J. L. (1988). A functional mRNA polyadenylation signal is required for transcription termination by RNA polymerase II. Genes Dev 2, 440-452.
Corbino, K. A., Barrick, J. E., Lim, J., Welz, R., Tucker, B. J., Puskarz, I., Mandal, M., Rudnick, N. D., and Breaker, R. R. (2005). Evidence for a second class of S-adenosylmethionine riboswitches and other regulatory RNA motifs in alpha-proteobacteria. Genome Biol. 6, R70.
Cromie, M. J., Shi, Y., Latifi, T., and Groisman, E. A. (2006). An RNA sensor for intracellular Mg(2+). Cell 125, 71-84.
Czechowski, T., Stitt, M., Altmann, T., Udvardi, M. K., and Scheible, W. R. (2005). Genome-wide identification and testing of superior reference genes for transcript normalization in Arabidopsis. Plant Physiol. 139, 5-17.
Davis R. H. Neurospora: Contributions of a model organism. Oxford University Press, New York, N.Y. (2000).
Dye, M. J., and Proudfoot, N. J. (2001). Multiple transcript cleavage precedes polymerase release in termination by RNA polymerase II. Cell 105, 669-681.
Ebbole, D. & Sachs, M. S. A rapid and simple method for isolation of Neurospora crassa homokaryons using microconidia. Fungal Genet. Newsl. 37, 17-18 (1990).
Eddy, S. R. & Durbin, R. RNA sequence analysis using covariance models. Nucleic Acids Res. 22, 2079-2088 (1994).
Eddy, S. R. INFERNAL. Version 0.55. Distributed by the author. Department of Genetics, Washington University School of Medicine. St. Louis, Mo.
Edwards, T. E. & Ferré-D'Amaré, A. R. Crystal structures of the Thi-box riboswitch bound to thiamine pyrophosphate analogs reveal adaptive RNA-small molecule recognition. Structure 14, 1459-1468 (2006).
Faou, P. & Tropschug, M. A novel binding protein for a member of CyP40-type Cyclophilins: N. crassa CyPBP37, a growth and thiamine regulated protein homolog to yeast Thi4p. J. Mol. Biol. 333, 831-844 (2003).
Faou, P. & Tropschug, M. Neurospora crassa CyPBP37: a cytosolic stress protein that is able to replace yeast Thi4p function in the synthesis of vitamin B1. J. Mol. Biol. 344, 1147-1157 (2004).
Fasken, M. B., and Corbett, A. H. (2005). Process or perish: quality control in mRNA biogenesis. Nat. Struct. Mol. Biol. 12, 482-488.
Froehlich, A. C., Loros, J. J. & Dunlap, J. C. Rhythmic binding of a WHITE COLLAR-containing complex to the frequency promoter is inhibited by FREQUENCY. Proc. Natl. Acad. Sci. USA 100, 5914-5919 (2003).
Fuchs, R. T., Grundy, F. J. & Henkin, T. M. The S(MK) box is a new SAM-binding RNA for translational regulation of SAM synthetase. Nat. Struct. Mol. Biol. 13, 226-233 (2006).
Galagan, J. E., et al. Sequencing of Aspergillus nidulans and comparative analysis with A. fumigatus and A. oryzae. Nature 438, 1105-1115 (2005).
Gelfand, M. S., Mironov, A. A., Jomantas, J., Kozlov, Y. I., and Perumov, D. A. (1999) A conserved RNA structure element involved in the regulation of bacterial riboflavin synthesis genes. Trends Genet. 15, 439-442.
Grundy, F. J., and Henkin, T. M. (1998). The S-box regulon: a new global transcription termination control system for methionine and cysteine biosynthesis genes in gram-positive bacteria. Mol. Microbiol. 30, 737-749.
Gusarov, I., and Nudler, E. (1999). The mechanism of intrinsic transcription termination. Mol. Cell. 3, 495-504.
Hagen, R., and Willmitzer, L. (1992). Transgenic potato plants depleted for the major tuber protein patatin via expression of antisense RNA. Plant Sci 87, 45-54.
Johansen, L. K., and Carrington, J. C. (2001). Silencing on the spot. Induction and suppression of RNA silencing in the Agrobacterium-mediated transient expression system. Plant Physiol. 126, 930-938.
Kertesz, S., Kerenyi, Z., Merai, Z., Bartos, I., Palfy, T., Barta, E., and Silhavy, D. (2006). Both introns and long 3′-UTRs operate as cis-acting elements to trigger nonsense-mediated decay in plants. Nucleic Acids Res. 34, 6147-6157.
Kim, D.-S., Gusti, V., Pillai, S. G. & Gaur, R. K. An artificial riboswitch for controlling pre-mRNA splicing. RNA 11, 1667-1677 (2005)
Kline, D. J., and Ferré-D'Amaré, A. R. (2006). Structural basis of glmS ribozyme activation by glucosamine-6-phosphate. Science 313, 1752-1756.
Kubodera, T., et al., Thiamine-regulated gene expression of Aspergillus oryzae thiA requires splicing of the intron containing a riboswitch-like domain in the 5′-UTR. FEBS Lett. 555, 516-520 (2003).
Lang, D., Eisinger, J., Reski, R., and Rensing, S. A. (2005). Representation and high-quality annotation of the Physcomitrella patens transcriptome demonstrates a high proportion of proteins involved in metabolism in mosses. Plant Biol. (Stuttg) 7, 238-250.
Logan, J., Falck-Pedersen, E., Darnell, J. E., Jr., and Shenk, T. (1987). A poly(A) addition site and a downstream termination region are required for efficient cessation of transcription by RNA polymerase II in the mouse beta maj-globin gene. Proc Natl Acad Sci USA 84, 8306-8310.
Loros, J. J. & Dunlap, J. C. Neurospora crassa clock-controlled genes are regulated at the level of transcription. Mol. Cell. Biol. 11, 558-563 (1991).
Mandal, M. & Breaker, R. R. Gene regulation by riboswitches. Nature Rev. Mol. Cell Biol. 5, 451-463 (2004).
Matlin, A. J., Clark, F. & Smith, C. W. Understanding alternative splicing: towards a cellular code. Nat. Rev. Mol. Cell Biol. 6, 386-398 (2005).
Maundrell, K. nmt1 of fission yeast: a highly expressed gene completely repressed by thiamine. J. Biol. Chem. 265, 10857-10864 (1989).
McColl, D., Valencia, C. A. & Vierula, P. J. Characterization and expression of the Neurospora crassa nmt-1 gene. Curr. Genet. 44, 216-223 (2003).
Mehra, A., Morgan, L., Bell-Pedersen, D., Loros, J. & Dunlap, J. C. Watching the Neurospora Clock Tick. Abstract in: Soc. Res. Biol. Rhythms, Amelia Island, Fla., Society for Research on Biological Rhythms 27 (2002).
Mironov, A. S., et al. Sensing small molecules by nascent RNA: a mechanism to control transcription in bacteria. Cell 111, 747-756 (2002).
Montange, R. K. & Batey, R. T. Structure of the S-adenosylmethionine riboswitch regulatory mRNA element. Nature 441, 1172-1175 (2006).
Muhlrad, D., and Parker, R. (1999). Aberrant mRNAs with extended 3′ UTRs are substrates for rapid degradation by mRNA surveillance. RNA 5, 1299-1307.
Murashige, T., and Skoog, F. (1962). A revised medium for rapid growth and bioassays with tobacco tissue cultures. Physiol. Plant 15, 473-497.
Nahvi, A., Barrick, J. E. & Breaker, R. R. Coenzyme B₁₂ riboswitches are widespread genetic control elements in prokaryotes. Nucleic Acids Res. 32, 143-150 (2004).
Nahvi, A., Sudarsan, N., Ebert, M. S., Zou, X., Brown, K. L. & Breaker, R. R. Genetic control by a metabolite binding mRNA. Chem. Biol. 9, 1043-1049 (2002).
Newman, T. C., Ohme-Takagi, M., Taylor, C. B., and Green, P. J. (1993). DST sequences, highly conserved among plant SAUR genes, target reporter transcripts for rapid decay in tobacco. Plant Cell 5, 701-714.
Orbach, M. J., Porro, E. B. & Yanofsky, C. Cloning and characterization of the gene for beta-tubulin from a benomyl-resistant mutant of Neurospora crassa and its use as a dominant selectable marker. Mol. Cell. Biol. 6, 2452-2461 (1986).
Proudfoot, N. (2004). New perspectives on connecting messenger RNA 3′ end formation to transcription. Curr. Opin. Cell Biol. 16, 272-278.
Proudfoot, N. J. (1989). How RNA polymerase II terminates transcription in higher eukaryotes. Trends Biochem. Sci. 14, 105-110.
Proudfoot, N. J., Furger, A., and Dye, M. J. (2002). Integrating mRNA processing with transcription. Cell 108, 501-512.
Rodionov, D. A., Vitreschak, A. G., Mironov, A. A. & Gelfand, M. S. Comparative genomics of thiamine biosynthesis in prokaryotes. J. Biol. Chem. 277, 48949-48959 (2002).
Rodionov, D. A., Vitreschak, A. G., Mironov, A. A., and Gelfand, M. S. (2002). Comparative genomics of thiamin biosynthesis in prokaryotes. New genes and regulatory mechanisms. J. Biol. Chem. 13, 48949-48959.
Romfo, C. M., Alvarez, C. J., van Heeckeren, W. J., Webb, C. J. & Wise, J. A. Evidence for splice site pairing via intron definition in Schizosaccharomyces pombe. Mol. Cell. Biol. 20, 7955-7970 (2000).
Roth, A., Winkler, W. C., Regulski, E. E., Lee, B. W. K., Lim, J., Jona, I., Barrick, J. E., Ritwik, A., Kim, J. N., Welz, R., et al. (2007). A riboswitch selective for the queuosine precursor preQᵢ contains an unusually small aptamer domain. Nat. Struct. Mol. Biol. 14, 308-317.
Seetharaman, S., Zivarts, M., Sudarsan, N. & Breaker R. R. Immobilized RNA switches for the analysis of complex chemical and biological mixtures. Nature Biotechnol. 19, 336-341 (2001).
Serganov, A. et al. Structural basis for discriminative regulation of gene expression by adenine- and guanine-sensing mRNAs. Chem. Biol. 11, 1-13 (2004).
Serganov, A., Polonskaia, A., Phan, A. T., Breaker, R. R. & Patel, D. J. Structural basis for gene regulation by a thiamine pyrophosphate-sensing riboswitch. Nature 441, 1167-1171 (2006).
Serganov, A., Yuan, Y. R., Pikovskaya, 0., Polonskaia, A., Malinina, L., Phan, A. T., Hobartner, C., Micura, R., Breaker, R. R., and Patel, D. J. (2004). Structural basis for discriminative regulation of gene expression by adenine- and guanine-sensing mRNAs. Chem. Biol. 11, 1729-1741.
Soukup, G. A. & Breaker, R. R. Relationship between internucleotide linkage geometry and the stability of RNA. RNA 5, 1308-1325 (1999).
Soukup, J. K., and Soukup, G. A. (2004). Riboswitches exert genetic control through metabolite-induced conformational change. Curr. Opin. Struct. Biol. 14, 344-349.
Sudarsan N., Barrick J. E. & Breaker R. R. Metabolite-binding RNA domains are present in the genes of eukaryotes. RNA 9, 644-647 (2003).
Sudarsan, N., Cohen-Chalamish, S., Nakamura, S., Emilsson, G. M., and Breaker, R. R. (2005). Thiamine pyrophosphate riboswitches are targets for the antimicrobial compound pyrithiamine. Chem. Biol. 12, 1325-1335.
Teixeira, A., Tahiri-Alaoui, A., West, S., Thomas, B., Ramadass, A., Martianov, I., Dye, M., James, W., Proudfoot, N. J., and Akoulitchev, A. (2004). Autocatalytic RNA cleavage in the human beta-globin pre-mRNA promotes transcription termination. Nature 432, 526-530.
Thore, S., Leibundgut, M. & Ban, N. Structure of the eukaryotic thiamine pyrophosphate riboswitch with its regulatory ligand. Science 312, 1208-1211 (2006).
Vader, A., Nielsen, H., and Johansen, S. (1999). In vivo expression of the nucleolar group I intron-encoded I-dirI homing endonuclease involves the removal of a spliceosomal intron. EMBO J. 18, 1003-1013.
Vann, D. C. Electroporation-based transformation of freshly harvested conidia of Neurospora crassa. Fungal Genet. Newsl. 42A, 53 (1995).
Vilela, C. & McCarthy, J. E. Regulation of fungal gene expression via short open reading frames in the mRNA 5′ untranslated region. Mol. Microbiol. 49, 859-867 (2003).
Vitreschak, A. G., Rodionov, D. A., Mironov, A. A. & Gelfand, M. S. Regulation of riboflavin biosynthesis and transport genes in bacteria by transcriptional and translational attenuation. Nucleic Acids Res. 30, 3141-3151 (2002).
Vitreschak, A. G., Rodionov, D. A., Mironov, A. A. & Gelfand, M. S. Regulation of the vitamin B₁₂ metabolism and transport in bacteria by a conserved RNA structural element. RNA 9, 1084-1097 (2003).
Voinnet, O., Rivas, S., Mestre, P., and Baulcombe, D. (2003). An enhanced transient expression system in plants based on suppression of gene silencing by the p19 protein of tomato bushy stunt virus. Plant J. 33, 949-956.
Wang, G., Ding, X., Yuan, M., Qiu, D., Li, X., Xu, C., and Wang, S. (2006). Dual function of rice OsDR8 gene in disease resistance and thiamine accumulation. Plant Mol. Biol. 60, 437-449.
Weinberg, Z., Barrick, J. E., Yao, Z., Roth, A., Kim, J. N., Gore, J., Wang, J. X., Lee, E. R., Block, K. F., Sudarsan, N. et al. (2007) Identification of 22 candidate structured RNAs in bacteria using Cmfinder comparative genomics pipline. (submitted).
Welz, R. & Breaker, R. R. Ligand binding and gene control characteristics of tandem riboswitches in Bacillus anthracis. RNA 13, (Advance Online Article) (2007).
Westergaard, M. & Mitchell, H. K. Neurospora V. A synthetic medium favoring sexual reproduction. Amer. J. Bot. 34, 573-577 (1947).
Winkler, W. C. & Breaker, R. R. Regulation of bacterial gene expression by riboswitches. Annu. Rev. Microbiol. 59, 487-517 (2005).
Winkler, W. C., Nahvi, A. & Breaker, R. R. Thiamine derivatives bind messenger RNAs directly to regulate bacterial gene expression. Nature 419, 952-956 (2002).
Yarnell, W. S., and Roberts, J. W. (1999) Mechanism of intrinsic transcription termination and antitermination. Science 284, 598-599.

1. A regulatable gene expression construct comprising
a nucleic acid molecule encoding an RNA comprising a riboswitch operably linked to a coding region, wherein the riboswitch regulates splicing of the RNA, wherein the riboswitch and coding region are heterologous, wherein regulation of splicing affects processing of the RNA.

2. The construct of claim 1, wherein the riboswitch regulates alternative spicing.

3. The construct of claim 1, wherein the riboswitch comprises an aptamer domain and an expression platform domain, wherein the aptamer domain and the expression platform domain are heterologous.

4. The construct of claim 1, wherein the RNA further comprises an intron, wherein the expression platform domain comprises a splice junction.

5. The construct of claim 4, wherein the splice junction is in the intron.

6. The construct of claim 4, wherein the splice junction is an alternative splice junction.

7. The construct of claim 4, wherein the splice junction is at an end of the intron.

8. The construct of claim 4, wherein the splice junction is active when the riboswitch is activated.

9. The construct of claim 4, wherein the splice junction is active when the riboswitch is not activated.

10. The construct of claim 1, wherein the riboswitch is activated by a trigger molecule.

11. The construct of claim 10, wherein the trigger molecule is TPP.

12. The construct of claim 1, wherein the riboswitch is a TPP-responsive riboswitch.

13. The construct of claim 1, wherein the riboswitch activates splicing of the intron.

14. The construct of claim 1, wherein the riboswitch activates alternative splicing.

15. The construct of claim 1, wherein the riboswitch represses splicing of the intron.

16. The construct of claim 1, wherein the riboswitch represses alternative splicing.

17. The construct of claim 1, wherein RNA has a branched structure.

18. The construct of claim 1, wherein the RNA is pre-mRNA.

19. The construct of claim 1, wherein the riboswitch is in the 3′ untranslated region of the RNA.

20. The construct of claim 4, wherein the intron is in the 3′ untranslated region of the RNA.

21. The construct of claim 4, wherein an RNA processing site is in the intron.

22. The construct of claim 21, wherein splicing of the intron removes the RNA processing site from the RNA thereby affecting processing of the RNA.

23. The construct of claim 22, wherein the affect on processing of the RNA comprises elimination of processing of the RNA mediated by the RNA processing site.

24. The construct of claim 22, wherein the affect on processing of the RNA comprises an alteration in transcription termination.

25. The construct of claim 22, wherein the affect on processing of the RNA comprises an increase in degradation of the RNA.

26. The construct of claim 22, wherein the affect on processing of the RNA comprises an increase in turnover of the RNA.

27. The construct of claim 4, wherein the riboswitch overlaps the 3′ splice junction of the intron.

28. The construct of claim 27, wherein splicing of the intron reduces or eliminates the ability of the riboswitch to be activated.

29. The construct of claim 3, wherein the region of the aptamer domain with splicing control is located in the P4 and P5 stem.

30. The construct of claim 29, wherein the region of the aptamer domain with splicing control is also located in loop 5.

31. The construct of claim 29, wherein the region of the aptamer domain with splicing control is also located in stem P2.

32. The construct of claim 3, wherein the splice site is located at a position between −130 to −160 relative to the 5′ end of the aptamer domain.

33. The construct of claim 3, wherein the RNA further comprises a second intron, wherein the 3′ splice site of the second intron is located at a position between −220 to −270 relative to the 5′ end of the aptamer domain.

34. The construct of claim 3, wherein the splice junction is a 5′ splice junction.

35. A method for affecting processing of RNA comprising introducing into the RNA a construct comprising a riboswitch, wherein the riboswitch is capable of regulating splicing of RNA, wherein the RNA comprises an intron, wherein regulation of splicing affects processing of the RNA.

36. The method of claim 35, wherein the riboswitch comprises an aptamer domain and an expression platform domain, wherein the aptamer domain and the expression platform domain are heterologous.

37. The method of claim 36, wherein the expression platform domain comprises a splice junction.

38. The method of claim 35, wherein the splice junction is in the intron.

39. The method of claim 37, wherein the splice junction is an alternative splice junction.

40. The method of claim 37, wherein the splice junction is at an end of the intron.

41. The method of claim 37, wherein the splice junction is active when the riboswitch is activated.

42. The method of claim 37, wherein the splice junction is active when the riboswitch is not activated.

43. The method of claim 35, wherein the riboswitch is activated by a trigger molecule.

44. The method of claim 43, wherein the trigger molecule is TPP.

45. The method of claim 35, wherein the riboswitch is a TPP-responsive riboswitch.

46. The method of claim 35, wherein the riboswitch activates splicing.

47. The method of claim 35, wherein the riboswitch activates alternative splicing.

48. The method of claim 35, wherein the riboswitch represses splicing.

49. The method of claim 35, wherein the riboswitch represses alternative splicing.

50. The method of claim 35, wherein said splicing does not occur naturally.

51. The method of claim 36, wherein the region of the aptamer domain with splicing control is located in loop 5.

52. The method of claim 35, wherein the construct further comprises the intron.

53. The method of claim 35, wherein the riboswitch is in the 3′ untranslated region of the RNA.

54. The method of claim 35, wherein the intron is in the 3′ untranslated region of the RNA.

55. The method of claim 35, wherein an RNA processing site is in the intron.

56. The method of claim 55, wherein splicing of the intron removes the RNA processing site from the RNA thereby affecting processing of the RNA.

57. The method of claim 56, wherein the affect on processing of the RNA comprises elimination of processing of the RNA mediated by the RNA processing site.

58. The method of claim 56, wherein the affect on processing of the RNA comprises an alteration in transcription termination.

59. The method of claim 56, wherein the affect on processing of the RNA comprises an increase in degradation of the RNA.

60. The method of claim 56, wherein the affect on processing of the RNA comprises an increase in turnover of the RNA.

61. The method of claim 37, wherein the riboswitch overlaps the 3′ splice junction of the intron.

62. The method of claim 61, wherein splicing of the intron reduces or eliminates the ability of the riboswitch to be activated.

63. The method of claim 36, wherein the region of the aptamer domain with splicing control is located in stem P2.

64. The method of claim 36, wherein the splice site is located at a position between −130 to −160 relative to the 5′ end of the aptamer domain.

65. The method of claim 36, wherein the RNA further comprises a second intron, wherein the 3′ splice site of the second intron is located at a position between −220 to −270 relative to the 5′ end of the aptamer domain.

66. The method of claim 36, wherein the splice site is a 5′ splice site.

67. The method of claim 35 further comprising bringing into contact a trigger molecule for the riboswitch, thereby affecting processing of the RNA.
